(19) United States
(12) Patent Application Publication
(10) Pub. No.: US 2008/0275028 A1
(43) Pub. Date: Nov. 6, 2008

(54) Modulators or Alpha7 Nicotinic Acetylcholine Receptors and Therapeutic Uses Thereof

(76) Inventors: Giovanni Gaviraghi, Siena (IT); Chiara Ghiron, Siena (IT); Hendrik Bothmann, Siena (IT); Renza Roncarati, Siena (IT); Georg Christian Terstappenn, Siena (IT)

Correspondence Address: CHOATE, HALL & STEWART LLP, TWO INTERNATIONAL PLACE, BOSTON, MA, 02110, US

(21) Appl. No.: 11/632,545

(22) PCT Filed: Jul. 19, 2005
(86) PCT No.: PCT/EP05/07846

§ 371 Date: Jan. 23, 2008

Related U.S. Application Data
(60) Provisional application No. 60/589,003, filed on Jul. 20, 2004.

(51) Int. Cl. A61K 31/445; C07D 401/12; C07D 211/12; C07D 295/15; C07D 243/08; C07D 211/60; C07D 409/12; C07D 207/09; A61P 29/00; A61P 25/28; A61P 37/00; A61P 25/00; A61K 31/5375; A61K 31/55; A61K 31/551; A61K 31/496; A61K 31/495; A61K 31/4545

(52) U.S. Cl. 514/217.12; 540/597; 546/233; 544/165; 544/400; 540/575; 546/225; 544/360; 546/213; 548/567; 514/237.8; 514/218; 514/253.01; 514/255.01; 514/252.12; 514/318; 514/331

(57) ABSTRACT

The present invention relates to compounds with α7 nAChR agonistic activity, processes for their preparation, pharmaceutical compositions containing the same and the use thereof for the treatment of neurological, psychiatric, cognitive, immunological and inflammatory disorders.

The present invention relates to compounds with α7 nicotinic acetylcholine receptor (α7 nAChR) agonistic activity, processes for their preparation, pharmaceutical compositions containing the same and the use thereof for the treatment of neurological and psychiatric diseases.

### BACKGROUND OF THE INVENTION

A number of recent observations point to a potential neuroprotective effect of nicotine in a variety of neurodegeneration models in animals and in cultured cells, involving excitotoxic insults (1-5), trophic deprivation (6), ischemia (7), trauma (8), Aβ-mediated neuronal death (9-11) and protein-aggregation mediated neuronal degeneration (9;12). In many instances where nicotine displays a neuroprotective effect, a direct involvement of receptors comprising the α7 subtype has been invoked (7;11;13-16) suggesting that activation of α7 subtype-containing nicotinic acetylcholine receptors may be instrumental in mediating the neuroprotective effects of nicotine. The available data suggest that the α7 nicotinic acetylcholine receptor represents a valid molecular target for the development of agonists/positive modulators active as neuroprotective molecules. Indeed, α7 nicotinic receptor agonists have already been identified and evaluated as possible leads for the development of neuroprotective drugs (18-22). Involvement of α7 nicotinic acetylcholine receptor in inflammatory processes has also recently been described (23). Thus, the development of novel modulators of this receptor should lead to novel treatments of neurological, psychiatric and inflammatory diseases.

### SUMMARY OF THE INVENTION

The invention provides compounds acting as full or partial agonists at the α7 nicotinic acetylcholine receptor (α7 nAChR), pharmaceutical compositions containing the same compounds and the use thereof for the treatment of diseases that may benefit from the activation of the alpha 7 nicotinic acetylcholine receptor such as neurological and psychiatric disorders, in particular Alzheimer's disease and schizophrenia.

### DESCRIPTION OF THE INVENTION

In a first aspect, the invention provides a compound of formula I

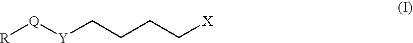

wherein:

Y is a group —CONH—; —NHCONH—; —NHCO—; —SO₂NH—; —NHSO₂—; —NHSO₂NH—; —OCONH; —NHCOO—

Q is a 5 to 10-membered aromatic or heteroaromatic ring

R is hydrogen; halogen; linear, branched or cyclic (C₁-C₆) alkyl, haloalkyl, alkoxy or acyl; hydroxy; cyano; nitro; mono- or di- (C₁-C₆) alkylamino, acylamino or alkylaminocarbonyl; carbamoyl; (C₆-C₁₀) aryl- or (C₁-C₆) alkylsulphonylamino; (C₆-C₁₀) aryl- or (C₁-C₆) alkylsulphamoyl; a 5 to 10-membered aromatic or heteroaromatic ring optionally substituted with: halogen; linear, branched or cyclic (C₁-C₃) alkyl, haloalkyl, alkoxy or acyl; hydroxy; cyano; nitro; amino; mono- or di- (C₁-C₆) alkylamino, acylamino or alkylaminocarbonyl groups; carbamoyl; (C₆-C₁₀) aryl- or (C₁-C₆) alkylsulphonylamino; (C₆₋₁₀) aryl- or (C₁-C₆) alkylsulphamoyl;

X is a group of formula

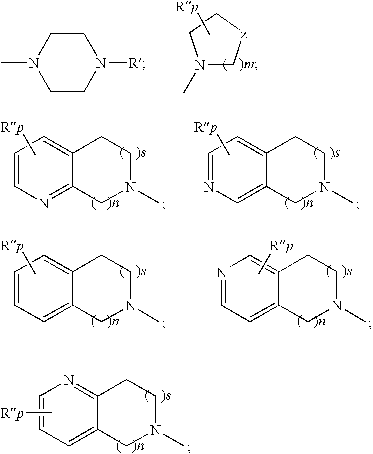

wherein

R¹ represents (C₁-C₆) acyl; linear, branched or cyclic (C₁-C₆) alkyl; a —(CH₂)ⱼ—R′″ group, wherein j=0,1 and R′″ is a 5 to 10-membered aromatic or heteroaromatic ring optionally substituted with: halogen; hydroxy; cyano; nitro; (C₁-C₆) alkyl, haloalkyl, alkoxy, acyl, acylamino groups;

Z is CH₂, N or O

m is an integer from 1 to 4

n is 0 or 1;

s is 1 or 2;

p is 0, 1 or 2;

R″, independently from one another for p=2, represents hydrogen; halogen; hydroxy; cyano; nitro; linear, branched or cyclic (C₁-C₆) alkyl, haloalkyl, alkoxy, acyl; a —(CH₂)ⱼ—R′″ group, wherein n and R′″ are as above defined; carbamoyl; (C₆-C₁₀) aryl- or (C₁-C₃) alkylsulphonylamino; (C₆-C₁₀) aryl- or (C₁-C₃) alkylsulphamoyl; mono- or di-[linear, branched or cyclic (C₁-C₆) alkyl]aminocarbonyl;

A first group (Ia) of preferred compounds of formula I are those in which:

Y is —CONH—; —NHCO—; —NHCONH—

Q is a 5 to 10-membered aromatic or heteroaromatic ring;

R is selected from the group consisting of hydrogen; halogen; linear, branched or cyclic (C₁-C₆) alkyl, alkoxy or alkylamino; trihaloalkyl; phenyl; naphthyl; pyridyl; pyrimidinyl; quinolinyl; isoquinolinyl; indolyl; thienyl; benzothienyl; furanyl; benzofuranyl; imidazolyl; benzoimidazolyl; pyrrolyl; optionally substituted as indicated above for the compounds of formula (I);

X is a group

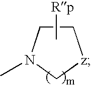

Z is CH₂, N or O

m is an integer from 1 to 4

p is 0, 1 or 2

R″, independently from one another for p=2, is selected from the group consisting of hydrogen; mono- or di-[linear, branched or cyclic (C₁-C₆) alkyl]aminocarbonyl; linear, branched or cyclic (C₁-C₆) alkyl, alkoxy, acyl;

Particularly preferred compounds Ia are those where Y is —CONH(Q)-;

Q is a 5 to 10-membered aromatic or heteroaromatic ring

R is selected from the group consisting of phenyl; naphthyl; pyridyl; pyrimidinyl; quinolinyl; isoquinolinyl; indolyl; thienyl; benzothienyl; furanyl; benzofuranyl; imidazolyl; benzoimidazolyl; pyrrolyl; optionally substituted as indicated above for the compounds of formula (I);

X is a group

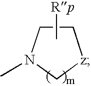

where

Z is CH₂, N or O

m is an integer from 1 to 4

p is 0, 1 or 2

R″, independently of one another for p=2, is selected from the group consisting of hydrogen; mono- or di-[linear, branched or cyclic (C₁-C₆) alkyl]aminocarbonyl; linear, branched or cyclic (C₁-C₆) alkyl, alkoxy, acyl;

Another group of particularly preferred compounds Ia are those where

Y is —NHCONH(Q)-;

Q is a 5 to 10-membered aromatic or heteroaromatic ring

R is selected from the group consisting of halogen; linear, branched or cyclic (C₁-C₆) alkyl, alkoxy or alkylamino; haloalkyl; phenyl; naphthyl; pyridyl; pyrimidinyl; quinolinyl; isoquinolinyl; indolyl; thienyl; benzothienyl; furanyl; benzofuranyl; imidazolyl; benzoimidazolyl; pyrrolyl; optionally substituted as indicated above for the compounds of formula (I);

X is a group

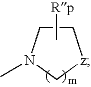

Z is CH₂, N or O

m is an integer from 1 to 4

is 0, 1 or 2

R″, independently from one another for p=2, is selected from the group consisting of hydrogen; mono- or di-[linear, branched or cyclic (C₁-C₆) alkyl]aminocarbonyl; linear, branched or cyclic (C₁-C₆) alkyl, alkoxy, acyl;

Another group of particularly preferred compounds Ia are those where

Y=—NHCO(Q)-;

Q is phenyl

R is selected from the group consisting of phenyl; naphthyl; pyridyl; pyrimidinyl; quinolinyl; isoquinolinyl; indolyl; thienyl; benzothienyl; furanyl; benzofuranyl; imidazolyl; benzoimidazolyl; pyrrolyl; optionally substituted as indicated above for the compounds of formula (I);

X is a group

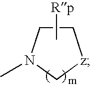

where

Z is CH₂, N or O

m is an integer from 1 to 4

p is 0, 1 or 2

R″, independently of one another for p=2, is selected from the group consisting of hydrogen; mono- or di-[linear, branched or cyclic (C₁-C₆) alkyl]aminocarbonyl; linear, branched or cyclic (C₁-C₆) alkyl, alkoxy, acyl;

A further group (Ib) of preferred compounds of formula (I) are those in which

Y is —CONH(Q)

Q is phenyl, indolyl

R is selected from the group consisting of halogen; phenyl; naphthyl; pyridyl; quinolinyl; isoquinolinyl; indolyl; thienyl; benzothienyl; furanyl; benzofuranyl; imidazolyl; benzoimidazolyl; pyrrolyl; optionally substituted as indicated above for the compounds of formula (I);

X is a group

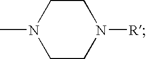

where R′ is a 5-10-membered aromatic or heteroaromatic ring optionally substituted with halogen or (C₁-C₆) alkoxy groups;

A further group (Ic) of preferred compounds of formula (I) are those in which

Y is —NHCONH(Q)

Q is phenyl, indolyl

R is selected from the group consisting of halogen; phenyl; naphthyl; pyridyl; quinolinyl; isoquinolinyl; indolyl; thienyl; benzothienyl; furanyl; benzofuranyl; imidazolyl; benzoimidazolyl; pyrrolyl; optionally substituted as indicated above for the compounds of formula (I);

X is a group

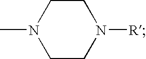

where R′ is a 6-membered aromatic or heteroaromatic ring optionally substituted with halogen or (C₁-C₆) alkoxy groups;

Another group (Id) of preferred compounds of formula I are those in which

Y is —NHCO(Q);

Q is phenyl, pyridyl

R is selected from the group consisting of phenyl; naphthyl; pyridyl; quinolinyl; pyrimidinyl; isoquinolinyl; indolyl; thienyl; benzothienyl; furanyl; benzofuranyl; imidazolyl; benzoimidazolyl; pyrrolyl; optionally substituted as indicated above for the compounds of formula (I);

X is a group

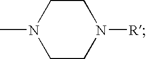

where R′ is a phenyl ring optionally substituted with halogen or (C₁-C₆) alkoxy groups;

Particularly preferred are the compounds (Id) wherein

Y is —NHCO(Q);

Q is phenyl

R is selected from the group consisting of phenyl; pyridyl; indolyl; pyrimidinyl; optionally substituted with: halogen; linear, branched or cyclic (C₁-C₃) alkyl, alkoxy or acyl; cyano; (C₁-C₆) alkylamino; acylamino; alkylaminocarbonyl groups; carbamoyl;

X is a group

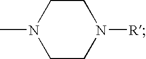

where R′ is a phenyl ring optionally substituted with halogen or (C₁-C₆) alkoxy groups

The compounds of the invention can be in the form of free bases or acid addition salts, preferably salts with pharmaceutically acceptable acids. The invention also includes separated isomers and diastereomers of compounds I, or mixtures thereof (e.g. racemic mixtures).

The compounds of Formula (I) can be prepared through a number of synthetic routes amongst which the ones illustrated in Schemes 1, 2, and 3 (see also for reference Bioorg. Med. Chem. Lett. 1995, 5 (3), 219-222).

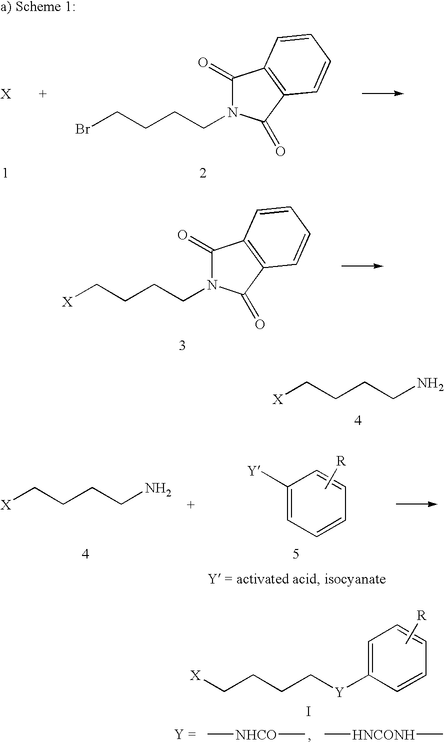

According to Scheme 1, a suitably activated butylphthalimide (compound 2) is reacted with an amine (compound 1) in an organic solvent in the presence of a base. For example, a mixture of 1 (or its hydrochloride salt) and 2 are refluxed in methylethyl ketone in the presence of alkaline carbonate until the reaction is complete, then the reaction mixture is cooled, the insoluble materials removed by filtration, the filtrate washed with CHCl₃, and the filtrate and washings concentrated to dryness.

In the following step, the N-(4-aminobutyl)phthalimide 3 is converted into a (4-aminobutyl)amine 4, for example by refluxing a mixture of 3 and hydrazine hydrate in ethanol. Then 4 is reacted with an activated species 5 such as for example (but not limited to) an acid chloride or an isocyanate in an organic solvent in the presence of a base. For example, to a mixture of 4 and 5 in CH₂Cl₂ triethylamine and a catalytic amount of DMAP are added, to give compounds I. Alternatively, a mixture of 4, 5, a carbodiimide or carbonyldiimidazole and DMAP are reacted to yield compounds I.

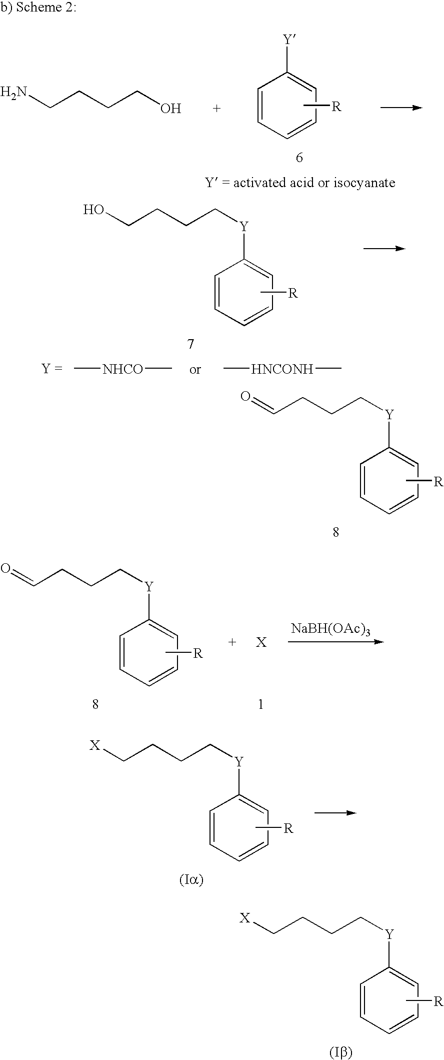

According to Scheme 2, aminobutanol is reacted with an activated acid species or an isocyanate—for example (but not limited to) a substituted acid chloride 6 in the presence of a base—in an organic solvent like dichloromethane until the reaction is complete. The alcohol 7 thus obtained is then oxidised under standard conditions (for example Swern oxidation) and aldehyde 8 is then reacted with the suitably substituted amine 1 under standard conditions—for example with sodium triacetoxyborohydride—to afford compound Iα. In the case of R being a halogen, Iα can be further processed—for example via a cross-coupling reaction with a boronic acid—to yield compound Iβ.

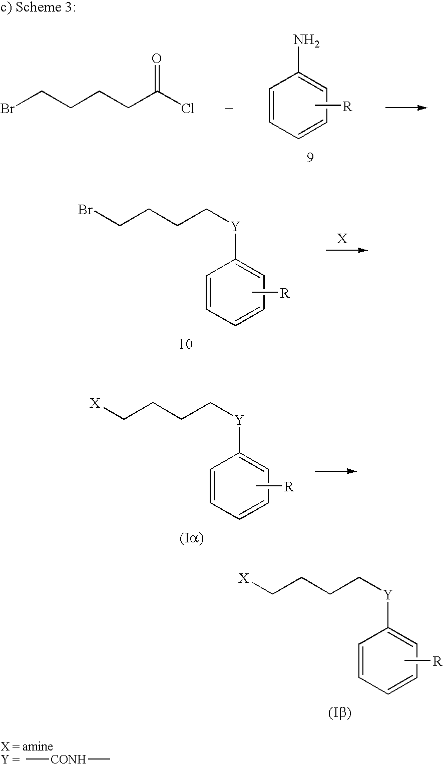

According to Scheme 3, 5-bromopentanoyl chloride is reacted with an (hetero)aromatic amine 9 in the presence of an organic base to afford a 5-bromopentanoic acid amide 10. This species is reacted with an amine 1 to displace the halogen and furnish compounds Iα. In the case of R being a halogen, Ia can be further processed—for example via a cross-coupling reaction with a boronic acid—to yield compounds Iβ.

The compounds of formula I, their optical isomers or diastereomers can be purified or separated according to well-known procedures, including but not limited to chromatography with chiral matrix and fractional crystallisation.

The pharmacological activity of a representative group of compounds of formula I was demonstrated in an in vitro assay utilising cells stably transfected with the alpha 7 nicotinic acetylcholine receptor and cells expressing the alpha 1 and alpha 3 nicotinic acetylcholine receptors and 5HT3 receptor as controls for selectivity. Neuroprotection of these compounds was demonstrated in a cell-based excitotoxicity assay utilising primary neuronal cell cultures.

According to a further aspect, the invention is therefore directed to a method of treating neurological and psychiatric disorders, which comprises administering to a subject, preferably a human subject in need thereof, an effective amount of a compound of formula I. Neurological and psychiatric disorders that may benefit from the treatment with the invention compounds include but are not limited to senile dementia, attention deficit disorders, Alzheimer's disease and schizophrenia. In general, the compounds of formula I can be used for treating any disease condition, disorder or dysfunction that may benefit from the activation of the alpha 7 nicotinic acetylcholine receptor, including but not limited to Parkinson's disease, Huntington's chorea, amyotrophic lateral sclerosis, multiple sclerosis, epilepsy, memory or learning deficit, panic disorders, cognitive disorders, depression, sepsis, arthritis, immunological and inflammatory disorders.

The dosage of the compounds for use in therapy may vary depending upon, for example, the administration route, the nature and severity of the disease. In general, an acceptable pharmacological effect in humans may be obtained with daily dosages ranging from 0.01 to 200 mg/kg.

In yet a further aspect, the invention refers to a pharmaceutical composition containing one or more compounds of formula I, in association with pharmaceutically acceptable carriers and excipients. The pharmaceutical compositions can be in the form of solid, semi-solid or liquid preparations, preferably in form of solutions, suspensions, powders, granules, tablets, capsules, syrups, suppositories, aerosols or controlled delivery systems. The compositions can be administered by a variety of routes, including oral, transdermal, subcutaneous, intravenous, intramuscular, rectal and intranasal, and are preferably formulated in unit dosage form, each dosage containing from about 1 to about 1000 mg, preferably from 1 to 600 mg of the active ingredient. The compounds of the invention can be in the form of free bases or as acid addition salts, preferably salts with pharmaceutically acceptable acids. The invention also includes separated isomers and diastereomers of compounds I, or mixtures thereof (e.g. racemic mixtures). The principles and methods for the preparation of pharmaceutical compositions are described for example in Remington's Pharmaceutical Science, Mack Publishing Company, Easton (Pa.).

### DESCRIPTION OF THE FIGURES

FIG. 1

Effect of compound from Example 64 on NMDA-induced toxicity in rat cortical neurons. Rat cortical neurons were pre-treated with the compound at the indicated concentrations 24 h before addition of NMDA and toxicity determined by lactate dehydrogenase (LDH) measurements after 24 h. Data of all experiments are normalised to 100% NMDA toxicity. Statistical analysis:

p<0.05 vs NMDA treatment; One-Way ANOVA and Tukey post test values were normalised to the level of NMDA (=100%).

FIG. 2

Effect of sub-chronic treatment of compound from Example 1 or nicotine on number of ChAT-positive neurons in the nucleus basalis of quisqualic acid injected animals. Compounds were administered 24 h and 1 h before quisqualic acid injection and for 7 days after lesioning. Doses: compound 3 mg/kg i.p. daily or nicotine 0.3 mg/kg i.p. daily. The doses were selected on the basis of literature data and comparable effects in behavioral studies. Number of neurons is expressed as % changes vs non-injected hemisphere. Statistical analysis: ANOVA and Fisher Post-Hoc test: F(3,21)=13.00 P<0.001*P<0.05 vs quisqualic acid injected rats # P<0.05 vs nicotine treated rats.

FIG. 3

FIG. 3a—Results of passive avoidance test

Effect of acute administration of compound from Example 1 on scopolamine-induced amnesia in young rats in passive avoidance test and reversion by the selective alpha-7 antagonist MLA. Amnesia was induced by scopolamine 0.5 mg/kg i.p. 20 min before training trial and the compound (3 mg/kg i.p.) was injected 5 min after scopolamine. MLA (5 mg/kg i.p.) was administered 10 min before scopolamine and compound administration. Results are presented as retest latencies 24 h after the training trial.

Statistical analysis: ANOVA and Tukey Post-Hoc test: * P<0.05 vs saline and scopolamine-treated rats # P<0.05 vs saline treated rats.

FIG. 3b—Results of object recognition test

Effect of acute administration of compound from Example 1 on scopolamine-induced amnesia in young rats. Amnesia was induced by scopolamine 0.2 mg/kg i.p. 20 min before training trial and the compound (3 mg/kg i.p.) was injected 5 min after scopolamine. Results are presented as discrimination index calculated on the exploration time of new (N) and familiar (F) objects during the test trial performed after 2 h from the training trial as follow: Discrimination index: N−F/N+F. Statistical analysis: ANOVA and Tukey Post-Hoc test: * P<0.05 scopolamine-treated rats.

### EXPERIMENTAL PROCEDURES—SYNTHESIS OF COMPOUNDS

General

Unless otherwise specified all nuclear magnetic resonance spectra were recorded using a Bruker AC200 (200 MHz) or a Varian Mercury Plus 400 Mhzspectrometer equipped with a PFG ATB Broadband probe.

HPLC-MS analyses were performed with an Agilent 1100 instrument, using a Zorbax Eclipse XDB-C8 4.6×150 mm; a Zorbax CN 4.6×150 mm column or a Zorbax Extend C18 2.1×50 mm column, coupled to an atmospheric API-ES MS for the 2.5 minutes method. The 5 and 10 minute methods were run using a waters 2795 separation module equipped with a Waters Micromass ZQ (ES ionisation) and Waters PDA 2996, using a Waters XTerra MS C18 3.5 μm 2.1×50 mm column.

Preparative HLPC was run using a Waters 2767 system with a binary Gradient Module Waters 2525 pump and coupled to a Waters Micromass ZQ (ES) or Waters 2487 DAD, using a Supelco Discovery HS C18 5.0 μm 10×21.2 mm column

Gradients were run using 0.1% formic acid/water and 0.1% formic acid/acetonitrile with gradient 5/95 to 95/5 in the run time indicated.

All column chromatography was performed following the method of Still, C.; J. Org Chem 43, 2923 (1978). All TLC analyses were performed on silica gel (Merck 60 F254) and spots revealed by UV visualisation at 254 nm and KmnO4 or ninhydrin stain.

All microwave reactions were performed in a CEM Discover oven.

### N-(4-(Arylpiperazin-1-yl)-butyl)phthalimides

The compounds were prepared following the general procedure outlined in Nishikawa, Y.; et al; Chem. Pharm. Bull., 1989, 37 (1), 100-105.

A mixture of N-(4-bromobutyl)-phthalimide (0.00135 mol), 1-(aryl)-piperazine hydrochloride (0.00135 mol), K₂CO₃ (0.00270 mol), NaI (0.00186 mol) and methylethyl ketone (7 mL) was refluxed for 20 h with stirring. After the mixture was cooled, the insoluble materials were removed by filtration and washed with CHCl₃. The filtrate and the washings were concentrated to dryness in vacuo.

The residue was subjected to chromatography on silica gel using CHCl₃/MeOH 95/5 as eluent.

### 4-[4-(Aryl-piperazin-1-yl)]-butylamines

A solution of N-(4-(Arylpiperazin-1-yl)-butyl)phthalimides (0.236 mmol) and hydrazine hydrate (0.478 mmol) in ethanol (2 mL) was refluxed for 2 h with stirring. After the solution had cooled, the insoluble materials were removed by filtration and washed with EtOH. The filtrate and the washings were concentrated to dryness in vacuo. The residue was taken up with CHCl₃. The CHCl₃ layer was washed with water, dried and concentrated to give the title amine.

### 4-[4-(2-Methoxy-phenyl)-piperazin-1-yl]-butylamine

a) Following the general procedure, 2-methoxyphenyl-piperazine (3.4 mL, 17.7 mmol) is added to a suspension of N-(4-bromobutyl)phthalimide (5 g, 17.7 mmol), sodium iodide (1.33 g, 8.85 mmol) and potassium carbonate (3.67 g, 26.6 mmol) in 2-butanone (70 mL). The resulting suspension is stirred for 18 h at 100° C., before LC-MS check. The reaction is filtered and the solvent removed by vacuum distillation; the resulting oil is dissolved in 5% MeOH in dichloromethane, washed with water and sat. NaCl, dried over Na₂SO₄. The solvent is removed under reduced pressure to yield the desired product as a thick yellow oil. The residue is extracted into ethyl acetate and washed with water and then saturated brine and dried over sodium sulphate. The solvent is removed under reduced pressure to afford 5.01 g of 2-{4-[4-(2-methoxy-phenyl)-piperazin-1-yl]-butyl}-isoindole-1,3-dione used without further purification in step b) below (72%).

2-{4-[4-(2-Methoxy-phenyl)-piperazin-1-yl]-butyl}-isoindole-1,3-dione (5.01 g, 12.7 mmol) is dissolved in abs. EtOH (60 mL) and hydrazine monohydrate (2.54 mL, 26 mmol) is added dropwise. The reaction is heated at 100° C. for 1 h; the reaction is filtered, concentrated at reduced pressure and transformed into its hydrochloride salt. The salt is dissolved in 15% NaOH and extracted into ethyl acetate to yield 2.04 g of 4-[4-(2-Methoxy-phenyl)-piperazin-1-yl]-butylamine as waxy solid (7.8 mmol, 61%).

C₁₅H₂₅N₃O Mass (calculated) [263.39]; (found) [M+H+]32 264.39

LC Rt=0.45, 92% (5 min method)

NMR (400 MHz, CDCl3): 1.48 (2H, m); 1.57 (2H, m); 2.42 (2H, m); 2.65 (4H, bs); 2.72 (2H, m); 3.1 (4H, bs); 3.86 (3H, s); 6.85 (1H, d); 6.97 (3H, m).

### 4-[4-(2,4-Difluoro-phenyl)-piperazin-1-yl]-butylamine

To a solution of N-(4-bromobutyl)phthalimide (5 g, 17.73 mmol) and 1-(2,4-difluoro-phenyl)-piperazine (17.73 mmol) in 2-butanone (100 mL), potassium carbonate (26.6 mmol) and potassium iodide (13.3 mmol) were added. The resulting mixture was heated at 90° C. overnight. After cooling the solution was filtered and evaporated to dryness. The residue was dissolved in dichloromethane (100 mL) and washed with water. The organic phase was dried over sodium sulphate and evaporated. This material was dissolved in ethanol (100 mL) and hydrazine (2 eq) was added. The solution was refluxed for 4 hours when a thick precipitate formed. Conc. HCl (5 mL) was then added and the mixture heated for a further hour. After cooling the solvent was evaporated and the residue dissolved in 2M HCl (100 mL). This solution was filtered and the aqueous filtrate evaporated again to dryness. The resulting residue was taken in isopropanol (30 mL) and filtered to give the hydrochloride salt of the required product. The salt was converted in the free amine by dissolution in NaOH (15% w/w) and extraction with dichloromethane. (2.6 g, 54%).

¹H-NMR (CDCl₃) δ 1.3 (br s, 2H), 1.46-1.58 (m, 4H), 2.41 (t, 2H), 2.62 (s, 4H), 2.73 (t, 2H), 3.05 (br s, 4H), 6.77-6.83 (m, 2H), 6.87-6.94 (m, 1H) (M+1) e/z 270

### 4-Morpholin-4-yl-butylamine

a) Following the general procedure, morpholine (1.7 mL, 20 mmol) is added to a suspension of N-(4-bromobutyl)phthalimide (5.36 g, 20 mmol), sodium iodide (1.5 g, 10 mmol) and potassium carbonate (5.53 g, 40 mmol) in 2-butanone (80 mL). The resulting suspension is stirred for 18 h at 100° C., before LC-MS check. The reaction is filtered and the solvent removed by vacuum distillation; the resulting oil is dissolved in 5% MeOH in dichloromethane, washed with water and sat. NaCl, dried over Na₂SO₄. The solvent is removed under reduced pressure to yield the desired product as a thick yellow oil. The residue was extracted into ethyl acetate and washed with water and then saturated brine and dried over sodium sulphate. The solvent was removed under reduced pressure to afford 5.7 g of 2-(4-Morpholin-4-yl-butyl)-isoindole-1,3-dione used without further purification in step b) below.

C₁₆H₂₀N₂O₃ Mass (calculated) [288.35]; (found) [M+H+]=289.36

Lc Rt=0.83, 95% (3 min method)

b) 4-Morpholin-4-yl-butyl-isoindole-1,3-dione (5.69 g, 19 mmol) is dissolved in abs. EtOH (95 mL) and hydrazine monohydrate (3.8 mL, 80 mmol) is added dropwise. The reaction is heated at 100° C. for 1 h; LC-MS show the reaction to be complete. The reaction is filtered, concentrated at reduced pressure and taken up with toluene and dichloromethane to remove excess phthalhydrazide; the crude amine is purified by SCX column, eluting with MeOH:dichloromethane 1:1 followed by 2 M NH3 in MeOH, to afford 1.46 g (9.2 mmol, 48%).

C₈H₁₈N₂O Mass (calculated) [158.25]; (found) [M+H+]=159.27

LC Rt=0.29, 96% (3 min method)

NMR (400 MHz, CD3OD): 1.51 (4H, m); 2.36 (2H, m); 2.46 (4H, s); 2.64 (2H, m); 3.68 (4H, m).

¹H-NMR (CDCl₃) δ 1.26 (br s, 2H), 1.44-1.57 (m, 4H), 2.35 (t, 2H), 2.44 (br s, 4H), 2.71 (t, 2H), 3.72 (m, 4H)

### 4-(4-Methyl-piperazin-1-yl)-butylamine

Prepared in analogous manner as 4-[4-(2,4-difluoro-phenyl)-piperazin-1-yl]-butylamine and obtained in yield=25%.

¹H-NMR (dmso-d6+D₂O) δ 1.53-1.61 (m, 2H), 1.66-1.74 (m, 2H), 2.80 (t, 2H), 2.85 (s, 3H), 3.17 (m, 2H), 3.38 (br s, 4H), 3.67 (br s, 4H); (M+1) e/z 172.

### 4-Piperidin-1-yl-butylamine

a) Following the general procedure, N-(4-bromobutyl)phthalimide (5.96 g, 20 mmol) was added to a suspension of piperidine (1.98 mL, 20 mmol), sodium iodide (1.5 g, 10 mmol) and potassium carbonate (4.15 g, 21 mmol) in 2-butanone (100 mL). The resulting suspension was stirred for 18 h at 85° C. The reaction was filtered and the solvent removed by vacuum distillation; the resulting oil was washed with water and recovered with dichloromethane. The solvent was removed under reduced pressure to afford 3.7 g of desired product as a white solid (yield: 65%).

C₁₇H₂₂N₂O₂ Mass (calculated) [286.38]; (found) [M+H+]=287

Lc Rt=0.97, 95% (5 min method)

NMR (400 MHz, CDCl3) 1.41 (2H, m), 1.49-1.59 (6H, m), 1.65-1.72 (2H, m), 2.15-2.35 (6H, m), 3.69-3.73 (6H, m), 7.69-7.74 (2H, m), 7.80-7.85 (2H, m).

b) 2-(4-Piperidin-1-yl-butyl)-isoindole-1,3-dione (3.7 g, 13 mmol) was dissolved in EtOH (50 mL) and hydrazine monohydrate (1.26 mL, 26 mmol) was added dropwise. The mixture was heated at 80° C. for 4 h. The reaction was filtered, concentrated at reduced pressure and taken up with toluene and dichloromethane to remove excess phthalhydrazide by filtration; the crude amine was purified by SCX column, eluting with MeOH:dichloromethane 1:1 followed by 2 M NH3 in MeOH, to afford g (410 mg, 35%).

C₉H₂₀N₂ Mass (calculated) [156.27]; (found) [M+H+]157

LC Rt=0.31 (5 min method)

NMR (400 MHz, CD3OD): 1.45-1.62 (10 H, m), 2.30-2.43 (10 H, m), 2.64-2.67 (2H, m).

### 1-(4-Amino-butyl)-piperidine-3-carboxylic acid diethylamide

a) Following the general procedure, commercially available N,N-diethylnipecotamide (3.4 g, 40 mmol) was weighed, placed in a flask and dissolved in 150 mL 2-butanone. To this N-(4-bromobutyl)phthalimide (11.3 g, 40 mmol), NaI (3 g, 20 mmol) and K2CO3 (8.28 g, 60 mmol) were added. The resulting mixture was heated at 85° C. for 20 hours. The solution was dried under vacuum and the crude solution was washed twice with water and dichloromethane. The organic layer was purified by flash chromatography using dichloromethane/MeOH 96/4.

C₂₂H₃₁N₃O₃ Mass (calculated) [385.50]; (found) [M+H+]=386

LC Rt=2.63, 94% (10 min method)

NMR (400 MHz, CDCl3): 1.08-1.12 (2H, m), 1.14-1.21 (2H, m), 1.52-1.76 (8H, m), 2.1 (1H, m), 2.23 (1 H, m), 2.44 (1H, m), 2.79 (1H, m), 2.94 (2H, m), 3.29-3.35 (4H, m), 3.69-3.73 (2H, m), 7.71-7.82 (2H, m), 7.82-7.86 (2H, m).

b) The phthalimide was deprotected using the general method described for the previous examples to obtain the desired product in 38% yield.

C₁₄H₂₉N₃O Mass (calculated) [255.23]; (found) [M+H+]32 256

LC Rt=0.35 (10 min method)

NMR (400 MHz, CDCl3): 1.09 (3H, m); 1.21 (3H, m); 1.50-1.60 (1H, m); 1.62-1.84 (6H, m), 2.13-2.19 (1H, m); 2.35-2.40 (1H, m); 2.46-2.50 (2H, m); 2.79-3.02 (5H, m); 3.27-3.47 (4H, m); 5.20-5.31 (3H, m).

General Procedure for the synthesis of biaryl carboxylic acids

Prepared according to the procedure outlined in Gong, Y. and Pauls, H. W. Synlett, 2000, 6, 829-831.

A catalytic amount of Pd(PPh₃)₄ was added to a degassed solution of 4-carboxyphenylboronic acid (0.001 mol) and arylic bromide (0.001 mol) in 0.4 M sodium carbonate solution (5 mL) and acetonitrile (5 mL).

The mixture was heated at 90° C. under N₂ for 15-20 h. The hot suspension was filtered. The filtrate was concentrated to about a half the original volume and then washed with CH₂Cl₂. The aqueous layer was acidified with conc. HCl and the resulting precipitate was collected.

### 2′-Amino-biphenyl-4-carboxylic acid

Yield: 80%

¹H-NMR (CD₃OD) δ (ppm): 8.10 (d, 1H); 7.50 (d, 2H); 6.94 (m, 4H)

Mass (ES) m/z %: 214 (M+1, 100%).

### 4-(Pyridin-2-yl)-benzoic acid

Yield: 70%;

¹H-NMR (CD₃OD) δ (ppm): 8.63 (d, 1H); 8.05 (m, 4H); 7.90 (m, 2H); 7.51 (m, 1H).

Mass (ES) m/z %: 200 (M+1, 100%).

### 4-(1-Oxy-pyridin-2-yl)-benzoic acid

Mass (ES) m/z %: 216 (M+1, 100%).

### 2′-Methylbiphenyl-4-carboxylic acid

Prepared with a modification of the procedure outlined in Leadbeater, N. E.; Marco, M; Org. Lett. 2002, 4 917) 2973-2976:

In a 10 mL glass tube were placed 4-carboxyphenyl boronic acid (166 mg, 1.0 mmol), 2-bromotoluene (120 μL, 1.0 mmol), Na₂CO₃ (315 mg, 3 mmol), Pd(OAc)₂ (1 mg, 0.004 mmol), 2 mL of water and a magnetic stirbar. The vessel was sealed with a septum and placed into the microwave cavity. Microwave irradiation (maximum emitted power 200W) was used to increase the temperature to 150° C.; the reaction mixture was then kept at this temperature for 5 min.

The mixture was allowed to cool to room temperature, and the reaction mixture was filtered washing with little CHCl₃. The aqueous layer was acidified, and the precipitate collected. The product was purified by chromatography on silica gel using Petroleum Ether/AcOEt 50/50 as eluent to give 67.8 mg of 12, yield 32%.

¹H-NMR (CD₃OD) δ (ppm): 8.05 (m, 2H, arom); 7,41 (m, 2H, arom); 7.21 (m, 4H, arom); 2.22 (s, 3H, C—CH₃).

Mass (ES) m/z %: 424 (2M, 100%).

### 2′-Nitrobiphenyl-4-carboxylic acid

To a stirred solution of 2′-aminobiphenyl-4-carboxylic acid (213 mg, 0.001 mol) in hexane/water/acetone (6.7:5:1, 6 mL), were added at 0° C. NaHCO₃ (400 mg) and Oxone (1.050 g). After 20 min a second portion of NaHCO₃ (400 mg) and Oxone® (1050 mg) was added and, after 20 min, a final portion of NaHCO₃ (400 mg) and Oxone® (1050 mg) was added. After 6 h the suspension was diluted with water and the organic layer was extracted with CH₂Cl₂. The combined organic layers were evaporated to give 2′-nitro-biphenyl-4-carboxylic acid (138.5 mg, 0.00057 mol), yield 57%.

¹H-NMR (CD₃OD) δ (ppm): 7.80 (m, 8H)

Mass (ES neg) m/z %: 242 (M−1, 100%); 226 (M−1-16, 70%)

### 2′-Methoxy-biphenyl-4-carboxylic acid

To a solution of 4-carboxyphenylboronic acid (3.32 g, 20 mmol), Fibrecat®1007 (2 g) and potassium carbonate (3.03 g, 22 mmol) in ethanol/water (20 mL/20 mL), 1-bromo-2-methoxy-benzene was added (4.11 g, 22 mmol). The reaction mixture was heated to reflux for 3 hours. After cooling, was filtered and the solution evaporated under reduced pressure. The residue was suspended in aq. citric acid (10% w/v), filtered and washed with water and diethyl ether. The resulting solid was dried under vacuum to yield the title compound (4.02 g, 88%).

¹H-NMR (dmso-d6) δ 3.79 (s, 3H), 7.08 (m, 1H), 7.34 (m, 1H), 7.58 (d, 1H), 7.96 (d, 1H)

### 2′-Chloro-biphenyl-4-carboxylic acid

A mixture of 4-carboxyphenylboronic acid (3.32 g, 20 mmol), Fibrecat®1007 (1 g), potassium carbonate (3.03 g, 22 mmol) and 1-bromo-2-chloro-benzene (4.2 g, 22 mmol) were exposed to microwave irradiation in a CEM Discovery Microwave for 15 minutes up to the maximum temperature of 120° C. After cooling, the mixture was filtered and the solution evaporated under reduced pressure. The residue was suspended in 1M HCl solution, filtered and washed with water and diethyl ether. The resulting solid was dried under vacuum to yield the title compound (4.0 g, 86%).

¹H-NMR (dmso-d6) δ 7.38-7.45 (m, 3H), 7.50-7.59 (m, 3H), 7.98-8.02 (m, 2H); (M+1) e/z 233

### 2′,4′-Difluoro-biphenyl-4-carboxylic acid

Prepared as outlined for 2′-chloro-biphenyl-4-carboxylic acid and obtained in yield=49%.

¹H-NMR (dmso-d6) δ 7.24 (m, 1H), 7.42 (m, 1H), 7.62-7.60 (m, 3H), 8.04 (d, 2H); (M+1) e/z 235

### 2′-Carbamoyl-biphenyl-4-carboxylic acid

Prepared as outlined for 2′-chloro-biphenyl-4-carboxylic acid and obtained in yield=29%.

¹H-NMR (dmso-d6) δ 7.33 (s, 1H), 7.40-7.52 (m, 6H), 7.70 (s, 1H), 7.95 (d, 2H); (M+1) e/z 242

### 2-Methyl-biphenyl-4-carboxylic acid

Prepared as outlined for 2′-chloro-biphenyl-4-carboxylic acid and obtained in yield=59%.

¹H-NMR (dmso-d6) δ 2.29 (s, 3H), 7.31-7.50 (m, 6H), 7.83 (dd, 1H), 7.89 (s, 1H); (M+1) e/z 213

### 6-Phenyl-nicotinic acid

Prepared as outlined for 2′-chloro-biphenyl-4-carboxylic acid

¹H-NMR (dmso-d6) δ 7.47-7.55 (m, 3H), 8.1 (d, 1H), 8.11-8.16 (m, 2H), 8.32 (dd, 1H), 9.13 (s, 1H), 13.39 (br s, 1H); (M+1) e/z 200

### 4-(5-oxo-4,5-dihydro-[1,2,4]oxadiazol-3-yl)-benzoic acid

### a) 4-(N-hydroxycarbamimidoyl)-benzoic acid methyl ester

A mixture of 4-cyano-benzoic acid methyl ester (16.5 g, 102 mmol), hydroxylamine hydrochloride (102 mmol), NaHCO₃ (110 mmol) in methanol (200 mL) was stirred for 30 minutes at room temperature and heated to the reflux for a further 3 hours. After cooling, water (400 mL) was added, the precipitate collected by filtration, washed and dried in a vacuum oven at 50° C. for 8 hours to give the title compound as a white solid (16.5 g, 83%). (M+1) e/z 195

### b) 4-(5-Oxo-4,5-dihydro-[1,2,4]oxadiazol-3-yl) -benzoic acid

To a solution of 4-(N-hydroxycarbamimidoyl)-benzoic acid methyl ester (5.7 g, 29.4 mmol) in dioxane (30 mL) was added CDI (1.2 eq). The reaction mixture was heated to 110° C. for 30 minutes. After cooling the solvent was evaporated, the residue suspended in water and the pH adjusted to pH=2 with aq. HCl (3M). The precipitate was collected by filtration washed with water, suspended in aqueous solution of NaOH (30 mL, 10% w/w) and methanol (50 mL) and left stirring at room temperature overnight. After evaporation of the solvents, the residue was taken in water (30 mL), pH adjusted to pH=2 adding aq. HCl (3M). The precipitate was collected by filtration, washed with water and dried under vacuum to yield the title compound as a white solid (4.1 g, 68%).

¹H-NMR (dmso-d6) δ 2.29 (s, 3H), 7.31-7.50 (m, 6H), 7.83 (dd, 1H), 7.89 (s, 1H); (M+1) e/z 213

### 4-(3-Methyl-[1,2,4]oxadiazol-5-yl)-benzoic acid

### a) N-(4-Methoxycarbonylbenzoyl)oxy)acetarnidine

To a solution of terephthalic acid monomethyl ester (5 g, 27.7 mmol) in dichloromethane (40 mL), CDI (27.7 mmol) was added. After 10 minutes stirring, N-hydroxy-acetamidine (27.7 mmol) was added and the resulting mixture stirred at room temperature for 3 hours. The solution was filtered and evaporated under reduced pressure to yield the title compound as a white solid (4.9 g, 75%).

(M+1) e/z 237

### b) 4-(3-Methyl-[1,2,4]oxadiazol-5-yl)-benzoic acid

A mixture of N-(4-methoxycarbonylbenzoyl)oxy)acetamidine (4.9 g, 20.7 mmol) and sodium acetate (20.7 mmol) in methanol (70 mL) and water (20 mL) was heated to 90° C. for 8 hours. After cooling a solid crystallised out of solution. The solid was filtered out, suspended in aq. NaOH solution (10% w/w, 30 mL) and methanol (30 mL) and left stirring at room temperature overnight. The solution was then evaporated under reduced pressure, the pH adjusted to pH=3 adding aq. HCl (6M). A precipitated formed, which was collected by filtration, washed with water, diethyl ether and dried under vacuum to yield the title compound as a white solid (2.5 g, 44%).

¹H-NMR (dmso-d6) δ 2.44 (s, 3H), 8.17 (m, 4H); (M+1) e/z 205

### 4-(1H-Tetrazol-5-yl)-benzoic acid

A mixture of 4-cyano-benzoic acid methyl ester (4.02 g, 25 mmol), sodium azide (32.5 mmol) and triethylamine hydrochloride (32.5 mmol) in toluene (40 mL) is heated at 97° C. for 7 hours. After cooling the solution, water (100 mL) was added. The aqueous phase was separated and to this solution HCl conc (7 g) was added. A precipitate formed which was isolated by filtration and washed with water. The obtained solid was suspended in aq. NaOH solution (20 mL, 10% w/w) and methanol (20 mL) and left stirring at room temperature for 2 hours. The solvent was then evaporated, water was added to the residue and the pH acidified with HCl (6M). A white precipitate formed which was isolated by filtration, washed with water and dried under vacuum to give the title compound (4.5 g, 95%).

¹H-NMR (dmso-d6) δ 8.09-8.17 (m, 4H); (M+1) e/z 191

### 4-(5-Methyl-[1,2,4]oxadiazol-3-yl)-benzoic acid

To a solution of 4-(N-hydroxycarbamimidoyl)-benzoic acid methyl ester (3.88 g, 20 mmol) in dichloromethane (20 mL), acetic anhydride (40 mmol) was added. The mixture was left stirring at room temperature overnight. After 16 hours the solvent was evaporated, pyridine (30 mL) was added and the reaction mixture heated at 95° C. for 2 days. After cooling the solution a solid crystallised out of solution. To this solution, water (20 mL) was added and after 2 hours stirring at room temperature it was filtered and the solid collected. The solid was suspended in aq. NaOH (30 mL, 10% w/w) and methanol (50 mL) and left stirring at room temperature overnight. After evaporation of the solvents, the residue was taken in water (30 mL), pH adjusted to pH=2 adding aq. HCl (3M). A precipitate formed which was collected by filtration, washed with water and dried under vacuum to yield the title compound as a white solid (3.8 g, 93%). (M+1) e/z 205.

General Procedure for the Synthesis of Biaryl-Carboxylic Acid Chlorides

The biarylcarboxylic acids (0.00057 mol) were treated with 5 mL of SOCl₂ for 5 h under reflux. The excess of SOCl₂ was removed by distillation and the crude acid chloride was used in the next reaction without further purification.

General Procedure for Acid—Amine Coupling Method using Acid Chlorides

A mixture of (4-aryl-piperazin-1-yl)-alkylamine (0.3 mmol), biarylcarboxylic acid chloride (0.3 mmol), triethylamine (0.56 mmol) and a catalytic amount of DMAP in CH₂Cl₂ was stirred at 0° C. for 10 min then at room temperature for 4 h.

The CH₂Cl₂ layer was washed with water, dried and concentrated. The residue purified by chromatography on silica gel with CHCl₃/MeOH 95/5 as eluent to give the title compound.

General Procedure for Acid—Amine Coupling Method using Carbodiimide

A solution of (4-aryl-piperazin-1-yl)-alkylamine (0.00014 mol) in 5 mL of dry CH₂Cl₂ was cooled to 0° C. The carboxylic acid (0.0002 mol), 1-(3-dimethylaminopropyl)-3-ethylcarbodiimide hydrochloride (EDC) (0.0002 mol) and a catalytic amount of DMAP were added and the reaction mixture was stirred at room temperature for 16 h.

The CH₂Cl₂ layer was then washed with water, dried and concentrated in vacuo and the residue purified by chromatography eluting with a gradient CHCl3/MeOH 99:1 to 95:5.

General Procedure for Acid—Amine Coupling Method using N,N′-carbonyldiimidazole (CDI)

To the preweighed acid (0.55 mmol), dimethylformamide was added (2 mL) to dissolve, followed by N,N′-carbonyldiimidazole (CDI) (0.55 mmol). The solution was then left for 60 minutes before adding the amine (0.6 mmol) and the reaction was stirred for a further 16 hours. The solvent was removed under reduced pressure and the crude mixture was treated with 5% MeOH in dichloromethane (2 mL) and washed with 10% sodium hydroxide solution (2 mL). This mixture was passed through a column packed with 5 grams of diatomaceous earth and the eluting the product with dichloromethane. The collected organic layer, containing the desired compound, was further purified using flash chromatography eluting with 10% MeOH in dichloromethane. Fractions containing the product were combined and the solvent removed under reduced pressure.

For less reactive carboxylic acids, activation was accomplished by heating the reaction at 60° C. for 2 h before adding the amine (1 eq) (1M solution in dimethylformamide) to the reaction mixture upon cooling; the reaction is then shaken at room temperature for 18-24 h.

Alternatively, to a solution of carboxylic acid (0.3 mmol) and CDI (0.3 mmol) in acetonitrile (3 mL), the amine (0.3 mmol) was added after 10 minutes. The reaction mixture was exposed to microwave irradiation for 10 minutes at 100° C. After cooling the reaction mixture was absorbed on a SCX cartridge, eluted with dichloromethane, methanol and methanol/ammonia solution. After evaporation, the residue was purified by silica column eluting with a gradient ethyl acetate/cyclohexane (1:1)ethyl acetate→ethyl acetate/methanol (9:1). The fractions containing the product were combined and the solvent evaporated.

General Procedure for Coupling of 4-oxo-butyl-benzamides via Reductive Alkylation

### a) 4-bromo-N-(4-hydroxybutyl)benzamide

A solution of 4-aminobutan-1-ol (20.71 g, 232 mmol) in dichloromethane (50 mL) was added to a stirring solution of 4-bromobenzoyl chloride (51 g, 232 mmol) in dichloromethane (250 mL). Diisopropylethylamine (40.4 mL, 232 mmol) was added and the colourless solution was stirred at room temperature. LC/MS indicated completion of the reaction after 50 mins. The solution was transferred to a separating funnel and washed with water. A white solid precipitated out which was filtered off and washed with dichloromethane to afford pure product. The filtrate was treated with H₂O which gave rise to further precipitate. The organic layer was washed with 1M HCl and NaHCO₃ (sat), dried over MgSO₄, filtered and concentrated in-vacuo to afford a further batch of product (total yield 57.99 g).

MS (ES) m/z 272/274 (Br)

### b) 4-Bromo-N-(4-oxobutyl)-benzamide

A solution of oxalyl chloride (4.15 mL, 47.6 mmol) in dichloromethane (200 mL) was stirred under a N₂ flow at −60° C. DMSO (6.76 mL, 95.2 mmol) was added cautiously ensuring that the temperature remained below −50° C. After 15 mins a solution of 4-bromo-N-(4-hydroxybutyl)benzamide (10 g, 36.6 mmol) in a mixture of dichloromethane (20 mL), THF (40 mL) and DMSO (5 mL) was added. After 30 mins the temperature had risen to −50° C. After 1 h triethylamine (1.637 g, 16.18 mmol) was added. The mixture was allowed to warm to room temperature and stirred overnight. LC/MS indicated completion of the reaction. H₂O (200 mL) was added to the reaction mixture. The organic layer was washed with 1M HCl, NaHCO₃ (sat) and brine, dried over MgSO₄, filtered and concentrated in-vacuo to afford an orange oil (9.93 g).

MS (ES) m/z 270/272 (Br); 252/254 (Br)

### a) 3-Bromo-N-(4-hydroxybutyl)benzamide

A solution of 4-aminobutan-1-ol (20.3 g, 228 mmol) in dichloromethane (50 mL) was added to a stirring solution of 3-bromobenzoyl chloride (50 g, 228 mmol) in dichloromethane (250 mL). DIPEA (39.6 mL, 228 mmol) was added and the colourless solution was stirred at room temperature. LC/MS indicated completion of the reaction after 50 mins. The solution was transferred to a separating funnel and washed with water. A white solid precipitated out which was filtered off and washed with dichloromethane to afford pure product. The filtrate was treated with H₂O which gave rise to further precipitate. The organic layer was washed with 1M HCl and NaHCO₃ (sat), dried over MgSO₄, filtered and concentrated in-vacuo to afford a further batch of product (total yield 46.82 g, 76%, 97% pure by LC/MS).

Rt=1.09; MS (ES) m/z 272/274 (Br)

### b) 3-Bromo-N-(4-oxo-butyl)-benzamide

A solution of oxalyl chloride (20.85 mL, 239 mmol) in dichloromethane (900 mL) was stirred under a N2 flow at −60° C. DMSO (33.9 mL, 478 mmol) was added cautiously ensuring that the temperature remained below −50° C. After 15 mins a solution of 3-bromo-N-(4-hydroxybutyl)benzamide 1 (50 g, 184 mmol) in a mixture of dichloromethane (100 mL), THF (400 mL) and DMSO (50 mL) was added. After 30 mins the temperature had risen to −50° C. After 1 h triethylamine (96.7 g, 956 mmol) was added. The mixture was allowed to warm to room temperature and stirred overnight. LC/MS indicated completion of the reaction. H₂O (1 L) was added to the reaction mixture. The organic layer was washed with 1M HCl, NaHCO₃ (sat) and brine, dried over MgSO4, filtered and concentrated in-vacuo to afford an orange oil (9.93 g, >100%, 97% pure by LC/MS).

Rt=1.18; MS (ES) m/z 252/254, 270/272 (Br)

### Reductive alkylation on N-(4-oxo-butyl)benzamides

To the preweighed amine (1 equivalent), the aldehyde was added dissolved in anhydrous dichloromethane (1.2 eq, dichloromethane). The solution was left to mix for 90 minutes before addition of sodium triacetoxyborohydride (1.5 equivalents). The reaction was left to mix for a further 16 hours. The crude reaction was then washed with saturated NaHCO₃ (2 mL solution/reaction) and the organic layer extracted. The dichloromethane crude solution was passed through an SCX column, eluting the desired product in 20% ammonia in methanol. Fractions containing the compound were combined and the product purified further using HPLC prep.

General Procedure for Suzuki coupling of N-(4-amino)butyl-3- or 4-bromobenzamides—Exemplified in Detail for N-(4-(4-acetylpiperazin-1-yl)butyl)-4-bromobenzamide and 2-ethylphenylboronic Acid

N-(4-(4-acetylpiperazin-1-yl)butyl)-4-bromobenzamide (86 mg, 0.225 mmol) was dissolved in DME:EtOH 1:1 (20 mL) and added to a microwave tube containing 2-ethylphenylboronic acid (34 mg, 0.225 mmol). 1M Na₂CO₃ in H₂O was added (300 μl, 0.3 mmol) followed by Pd(PPh₃)₄ (26 mg, 0.0225 mmol). The tube was capped, shaken by hand and loaded into the microwave for 10 mins at 150° C. The reaction was filtered through celite and washed with MeOH. The filtrate was concentrated in-vacuo and purified by reverse phase preparative HPLC. The product was taken on directly to form the HCl salt: 200 μl 1.25 M HCl in MeOH and 800 μl dichloromethane were added to the title compound and the solution was shaken and concentrated in-vacuo to afford the hydrochloride salt (38.7 mg).

MS (ES) m/z 408

General procedures for 5-alkylaminopentanoic Acid arylamides Preparation from 5-bromopentanoyl Chloride

In dichloromethane at 0° C.-room temperature. A solution of aromatic amine (1 eq) and triethylamine (1 eq) in dichloromethane (0.2 mmol/mL) is cooled at 0° C. under nitrogen atmosphere. 5-Bromopentanoyl chloride (1 eq) in dichloromethane (0.3 mmol/mL) is slowly added and the mixture stirred at room temperature for 1.5 hr. The amine (5 eq) and triethylamine (1 eq) are added at once and the reaction is stirred at room temperature for 40 hrs. The organic solution is then washed with brine, dried and the solvent removed. The product are crystallised by hexane: diethylether 1:1 or purified by flash chromatography.

Modified room temperature conditions for array synthesis: To a solution of aniline (1 eq) and triethylamine (1 eq) in dichloromethane (2 mL) at room temperature was slowly added 5-bromo-pentanoyl chloride (1 eq) and the mixture stirred for 1.5 hr. The solution was added to a previously prepared vial containing the amine (5 eq) and triethylamine (1 eq) and the reactions were shaken at room temperature for 40 hrs. The organic solution was washed with brine, dried and the solvent removed. The products were purified by flash chromatography or by preparative HPLC.

In dichloroethane/dimethylformamide at 55° C.: A substituted aromatic amine (1 eq) and triethylamine (1 eq) are weighed in a glass vial and 1,2-dichloroethane is added to give a 1.2 M solution; 5-bromovaleryl chloride (0.95 eq) is then added dropwise as a solution in dimethylformamide (1.2 M) and the reaction is shaken at room temperature for 1 h 30 min. The amine (3 eq) and triethylamine (1 eq) are then added as a solution in DCE (amine concentration 1.8 M) and the reaction mixture shaken at 55° C. for 4 h. After this period, the reaction mixture is cooled and partitioned between water and dichloromethane; the organic layer is washed with sat. NaCl and dried over Na₂SO₄. The crude amides obtained after solvent evaporation at reduced pressure are purified by preparative HPLC.

### 5-(4-Methyl-piperazin-1-yl)-pentanoic acid (4-bromo-phenyl)-amide

Prepared according the general procedure in dichloromethane at room temperature to give 3.7 g (70%) of the title compound.

C₁₆H₂₄N₃OBr Mass (calculated) [354.29]; found [M+H+]=354/356 (Br),

Lc Rt=0.58, 93%

NMR (400 MHz, DMSO): 1.43 (2H, m); 1.55 (2H, m); 2.23 (3H, s); 2.27-2.50 (12H, m); 7.44 (2H, d, J=9 Hz); 7.55 (2H, d, J=9 Hz); 10.05 (1H, s).

General Suzuki Cross-Coupling Procedure for the Synthesis of Arylamides

To a degassed mixture of 5-alkylamino-pentanoic acid bromoaryl-amide (0.1 g, 1 eq) and a substituted benzeneboronic acid (1.1 eq) in acetonitrile/sodium carbonate 0.4 M solution 1/1 (4 mL) a catalytic amount of Pd[(PPh₃)]₄ (5 mmol %) was added. The reaction mixture was heated at 90° C. for 20 minutes under microwave irradiation (150 Watt) and then again other 20 minutes. The organic layer was separated and purified by SCX column. The solvent was removed under reduced pressure to afford the corresponding product.

General Procedure for Urea Synthesis from Isocyanates

To a cooled 0.2 M solution of amine (1 eq) in dichloromethane, 1 eq of bromophenylisocyanate was added. The mixture was left stirring at 0° C. and it was stopped when a white solid was formed (1 h), after ca. 1 hour. The product was recovered by filtration as a white solid which was used without further purification.

General Suzuki Cross-Coupling Procedure for the Synthesis of Ureas Microwave Irradiation

To a degassed 0.067 M solution of bromide (1 eq, prepared following the procedure for ureas described above) in acetonitrile/water (1/1), the appropriate boronic acid (1 eq) and Na2CO3 (3 eq) were added followed by Pd[(PPh₃)]₄ (10% mol). The solution was irradiated under microwave conditions, using the following parameters: power=200 watt; ramp time=1 min; hold time=20 min; temp=90° C.; pressure=200 psi. The acetonitrile layer was separated and the crude mixture was purified using a SCX column washing with dichloromethane/MeOH followed by MeOH and then NH3/MeOH to elute the product. The fractions containing the desired product were combined and dried under reduced pressure.

Thermal Heating

The urea was weighted (1 eq, prepared following the procedure for ureas described above), placed in a 2-neck flask and dissolved in a degassed solution of acetonitrile/water (4/1, 0.04 M). To this solution boronic acid (1.1 eq), Na₂CO₃ (3 eq) and Pd[(PPh₃)]₄ (10% mmol) were added. The mixture was heated at 80° C. and stirred for 20 hours. The solution was filtered on Celite layer and purified using SCX or preparative HPLC.

### EXAMPLE 1

### N-{4-[4-(2,4-Dimethoxy-phenyl)-piperazin-1-yl]-butyl}-4- (pyridin-2-yl)-benzamide

### a) 1-(2,4-dimethoxy-phenyl)-piperazine hydrochloride

Prepared with a modification of Pascal, J. C.; et al. Eur. J. Med. Chem., 1990, 25, 291-293: a solution of 1.48 g (0.0097 mol) of 2,4-dimethoxyaniline, 1.89 g (0.0160 mol) of bis-2-chloroethylamine hydrochloride and 2.00 g of K₂CO₃ in 25 mL of 1-butanol was refluxed for 24 h then filtered hot.

The solvent was removed under reduced pressure and the residue triturated with acetone. The resulting powder was filtered and dried to give 1.25 g of the title compound.

¹H-NMR (DMSO-d₆) δ (ppm): 9.21 (br s, 1H); 6.82 (d, 1H); 6.52 (s, 1H); 6.42 (d, 1H); 3.74 (s, 3H); 3.68 (s, 3H); 3.12 (s, 4H); 3.07 (s, 4H).

### b) 2-{4-[4-(2,4-Dimethoxy-phenyl)-piperazin-1-yl]-butyl}-isoindole-1,3-dione

Prepared following the general procedure outlined in Nishikawa, Y.; et al; Chem. Pharm. Bull., 1989, 37 (1), 100-105.

A mixture of N-(4-bromobutyl)phthalimide (0.00135 mol), 1-(2′,4′-dimethoxyphenyl)-piperazine hydrochloride (0.00135 mol), K₂CO₃ (0.00270 mol), Nal (0.00186 mol) and methylethyl ketone (7 mL) was refluxed for 20 h with stirring. After the mixture had cooled, the insoluble materials were removed by filtration and washed with CHCl₃ The filtrate and the washings were concentrated to dryness in vacuo.

The residue was purified by cromatography on silica gel with CHCl₃/MeOH 95/5 as eluent. Yield: 68%.

¹H-NMR (CDCl₃) δ (ppm): 7.73 (m, 4H); 6.82 (d, 1H); 6.40 (m, 2H); 3.79 (s, 3H), 3.73 (s, 3H), 3.65 (m, 2H); 2.98 (m, 4H); 2.61 (m, 4H); 2.41 (t, 2H); 1.66 (m, 4H).

### c) 4-[4-(2,4-Dimethoxy-phenyl)-piperazin-1-yl]-butylamine

A solution of 2-{4-[4-(2,4-dimethoxy-phenyl)-piperazin-1-yl]-butyl}-isoindole-1,3-dione (0.000236 mol) and hydrazine hydrate (0.000478 mol) in ethanol (2 mL) was refluxed for 2 h with stirring. After the solution had cooled, any insoluble materials were removed by filtration and washed with EtOH. The filtrate and the washings were concentrated in vacuo to dryness. The residue was taken up with CHCl₃. The CHCl₃ layer was washed with water, dried and concentrated to give the title amine. Yield: 50%.

¹H-NMR (CDCl₃) δ (ppm): 6.85 (d, 1H); 6.41 (m, 2H); 3.81 (s, 3H); 3.75 (s, 3H); 3.01 (m, 4H); 2.63 (m, 4H); 2.40 (t, 2H); 1.35 (m, 6H).

### d) N-{4-[4-(2,4-Dimethoxy-phenyl)-piperazin-1-yl]-butyl}-4-(pyridin-2-yl)-benzamide

Prepared by reaction with 4-(pyridin-2-yl)-benzoic acid according to the general procedure (acid chloride method).

Yield: 35%.

Mp 154.5-156° C. (free base); 212-216° C. (HCl salt)

¹H-NMR (CDCl₃) δ (ppm): 8.66 (d, 1H); 8.02 (d, 2H); 7.85 (d, 2H); 7.75 (m, 2H); 7.23 (m, 1H); 6.96 (br s, 1H); 6.76 (d, 1H); 6.42 (d, 1H); 6.36 (dd, 1H); 3.78 (s, 3H); 3.72 (s, 3H); 3.47 (m, 2H); 2.97 (m, 4H); 2.65 (m, 4H); 2.47 (t, 2H); 1.70 (m, 4H)

Mass (ES) m/z %: 475 (M+1, 100%); 497 (M+Na, 19%)

HPLC: column Zorbax C8 MeOH 80%/H₂O 20%, 1.0 mL/min; Rt 6.54; area=99%

### EXAMPLE 2

### Biphenyl-4-carboxylic acid (4-[4-(2,4-dimethoxy-phenyl)-piperazin-1-yl]-butyl}-amide

Prepared from 4-[4-(2,4-dimethoxy-phenyl)-piperazin-1-yl]-butylamine and 4-biphenylcarboxylic acid following the general procedure (acid chloride method).

Yield: 35%

¹H-NMR (CDCl₃) δ (ppm): 7.82 (d, 2H); 7.5-7.6 (m, 4H); 7.48-7.5 (m, 3H); 6.89 (br s, 1H); 6.77 (d, 1H); 6.45 (d, 1H); 6.34 (dd, 1H); 3.80 (s, 3H); 3.73 (s, 3H); 3.49 (m, 2H); 2.96 (m, 4H); 2.64 (m, 4H); 2.45 (t, 2H); 1.68 (m, 4H).

Mass (ES) m/z %: 474 (M+1, 100%); 496 (M+Na, 6%).

HPLC: column: Zorbax CN AcCN 40%/H₂O (CF₃COOH pH=2.3) 60%, 0.8 mL/min; Rt=5.396; Area 98%

### EXAMPLE 3

### 2′-Nitro-biphenyl-4-carboxylic acid (4-[4-(2,4-dimethoxy-phenyl)-piperazin-1-yl]-butyl}-amide

Prepared from 4-[4-(2,4-dimethoxy-phenyl)-piperazin-1-yl]-butylamine and 2′-nitrobiphenyl-4-carboxylic acid following the general procedure (acid chloride method).

Yield: 17%

¹H-NMR (CDCl₃) δ (ppm): 7.7-7.9 (m, 3H); 7.45-7.55 (m, 2H); 7.3-7.4 (m, 3H); 6.84 (br s, 1H); 6.80 (d, 1H); 6.44 (d, 1H); 6.37 (dd, 1H); 3.80 (s, 3H); 3.74 (s, 3H); 3.49 (m, 2H); 2.97 (m, 4H); 2.63 (m, 4H); 2.46 (t, 2H); 1.68 (m, 4H)

Mass (ES) m/z %: 519 (M+1, 100%); 541 (M+Na, 11%)

HPLC: column Zorbax CN MeOH 50%/H₂O (CF₃COOH pH=2) 50%, 0.4 mL/min; Rt=17.209; Area 88%

### EXAMPLE 4

### 2′-Fluoro-biphenyl-4-carboxylic acid (4-[4-(2,4-dimethoxy-phenyl)-piperazin-1-yl]-butyl}-amide

Prepared from 4-[4-(2,4-dimethoxy-phenyl)-piperazin-1-yl]-butylamine and 2′-fluorobiphenyl-4-carboxylic acid following the general procedure (acid chloride method).

Yield: 20%

Mp=124-125.5° C.

Rₜ (CHCl₃/MeOH 95/5) 0.21

¹H-NMR (CDCl₃) δ (ppm): 7.81 (d, 2H); 7.56 (d, 2H); 7.1-7.4 (m, 4H); 6.99 (s br, 1H); 6.76 (d, 1H); 6.43 (d, 1H); 6.33 (dd, 1H); 3.78 (s, 3H); 3.71 (s, 3H); 3.46 (m, 2H); 2.94 (m, 4H); 2.60 (m, 4H); 2.44 (t, 2H); 1.66 (m, 4H)

Mass (ES) m/z %: 492 (M+1, 100%);

HPLC: column Zorbax CN AcCN 50%/H₂O (CF₃COOH pH=2,3) 50%, 0.4 mL/min; Rt=13.525; Area 96%

### EXAMPLE 5

### 2′-Methyl-biphenyl-4-carboxylic acid (4-[4-(2,4-dimethoxy-phenyl)-piperazin-1-yl]-butyl}-amide

Prepared from 4-[4-(2,4-dimethoxy-phenyl)-piperazin-1-yl]-butylamine and 2′-methylbiphenyl-4-carboxylic acid following the general procedure (acid chloride method).

Yield: 21%

¹H-NMR (CDCl₃) δ (ppm): 7.80 (d, 2H); 7.35 (d, 2H); 7.2-7.4 (m, 4H); 6.88 (br s, 1H); 6.79 (d, 1H); 6.46 (d, 1H); 6.36 (m, 1H); 3.82 (s, 3H); 3.76 (s, 3H); 3.50 (m, 2H); 2.98 (m, 4H); 2.66 (m, 4H); 2.47 (m, 2H); 2.25 (s, 3H); 1.70 (m, 4H)

Mass (ES) m/z %: 488 (M+1, 100%)

HPLC: column Zorbax C8 AcCN 40%/H₂O (CF₃COOH pH=2.3) 60%, 1.0 mL/min; Rt=11.748; Area 96%

### EXAMPLE 6

### N-{4-[4-(2-Methoxy-phenyl)-piperazin-1-yl]-butyl}-4-(pyridin-2-yl)-benzamide

### a) 2-{4-[4-(2-Methoxy-phenyl)-piperazin-1-yl]-butyl}-isoindole-1,3-dione

Prepared According to the General Procedure

Yield: 80%

1H-NMR (CDCl₃) δ (ppm): 7.72 (m, 4H); 6.89 (m, 4H); 3.81 (s, 3H); 3.69 (t, 2H); 3.15 (m, 4H); 2.60 (4H, m); 2.40 (t, 2H); 1.66 (m, 4H).

### b) 4-[4-(2-Methoxy-phenyl)-piperazin-1-yl]-butylamine

Prepared According to the General Procedure

Yield: 53%

¹H-NMR (CD₃OD) δ (ppm): 6.90 (m, 4H); 3.83 (s, 3H); 3.05 (m, 4H); 2.79 (t, 2H); 2.66 (4H, m); 2.43 (m, 2H); 1.60 (m, 4H).

Mass (ES) m/z %: 264 (M+1, 100%).

### c) N-{4-[4-(2-Methoxy-phenyl)-piperazin-1-yl]-butyl}-4-(pyridin-2-yl)-benzamide

Prepared by Reaction with 4-(pyridin-2-yl)-benzoic acid According to the General Procedure—Carbodiimide Method.

Yield: 41%

Mp=152.3-154.6° C.

Rₜ (CHCl₃/MeOH 95/5)=0.15

¹H-NMR (CDCl₃) δ (ppm): 8.66 (d, 1H); 8.00 (d, 2H); 7.84 (d, 2H); 7.70 (m, 2H); 7.21 (m, 1H); 6.8-7.0 (m, 5H); 3.80 (s, 3H); 3.44 (m, 2H); 3.03 (m, 4H); 2.62 (m, 4H); 2.43 (m, 2H); 1.65 (m, 4H).

Mass (ES) m/z %: 445 (M+1, 100%); 467 (M+Na, 78%).

HPLC: column Zorbax C8 MeOH 80%/H₂O 20%, 0.8 mL/min; Rt=4.72; area: 99.9%.

### EXAMPLE 7

### 1H-Indole-6-carboxylic acid (4-[4-(2,4-difluoro-phenyl)-piperazin-1-yl]-butyl}-amide

Following the general procedure, 6-indolecarboxylic acid (44 mg, 0.27 mmol) is dissolved in dimethylformamide (1 mL) and 1,1′-carbonyldiimidazole (44 mg, 0.27 mmol) is added. 4-[4-(2,4-Difluoro-phenyl)-piperazin-1-yl]-butylamine (73 mg, 0.27 mmol) dissolved in dimethylformamide (0.25 mL) is then added and the mixture is allowed to react for 18 h. Work-up followed by preparative HPLC affords the title compound (51 mg, 41%, >95% pure) as formate salt.

C₂₃H₂₆F₂N₄O Mass (calculated) [412.49]; (found) [M+H+]=413

LC Rt=3.02, 100% (10 min method)

NMR (400 MHz, CDCl3): 1.51 (4H, m); 2.34 (2H, t); 2.47 (4H, bs); 2.93 (4H, bs); 3.26 (2H, m); 6.49 (1H, s); 6.95-7.01 (2H, m); 7.12-7.17 (1H, m); 7.40 (2H, m); 7.6 (1H, dd, J=8.4, 1.2), 8.09 (1H, s); 8.17 (1H, HCOOH,s); 8.26 (1H, t); 11.27 (1H, s).

### EXAMPLE 8

### N-(4-Azepan-1-yl-butyl)-4-pyridin-2-yl-benzamide

### a) N-(4-Hydroxy-butyl)-4-pyridin-2-yl-benzamide

CDI (4.07 g, 25 mmol) was added to a solution of 4-pyridin-2-yl-benzoic acid (5.0 g, 25 mmol) in dichloromethane and the reaction mixture stirred for 4 hours. 4-aminobutanol (3.0 mL, 30 mmol) was added and the reaction mixture stirred for 4 hours after which the solution was washed with a saturated solution of Na₂CO₃. The organic layer was separated, dried over MgSO₄, filtered and the solvent removed under reduced pressure. The product was purified by column chromatography (dichloromethane, dichloromethane/MeOH 1%) to give 2.4 g of the title alcool.

LC Rt=0.98 min (5 min run)

(M+1=271)

1H NMR (400 MHz, DMSO): 8.71-8.66 (1H,m), 8.53-8.46 (1H, m), 8.78 (2H,d, 8.1 Hz), 8.12 (1H, d, 8.3 Hz), 7.94 (2H, d, 8.1 Hz), 7.92-7.83 (1H, m), 7.46-7.36 (1H, m), 4.38 (1H, t, 6.6 Hz), 3.42 (2H, dd, 6.6 Hz, 12.0 Hz), 3.35-3.25 (2H, m), 1.60-1.42 (4H,m).

### b) N-(4-Oxo-butyl)-4-pyridin-2-yl-benzamide

A solution of oxalyl chloride (42 μL, 0.48 mmol) in dichloromethane (5 mL) was stirred under N₂ at −60° C. DMSO (34 μL, 0.48 mmol) was added followed after 15 mins by a solution of alcohol (100 mg, 0.37 mmol) in dichloromethane (100 mL). After 2 h triethylamine (106 μl, 0.74 mmol) was added. The mixture was then allowed to warm to room temperature and stirred overnight. LC/MS indicated completion of the reaction. The organic layer was washed with a saturated solution of NH₄Cl, dried over MgSO₄, filtered and concentrated under reduced pressure to give 100 mg of a white powder (92% pure by LC/MS Rt=0.98, M+1=269) which was used in the next step without further purification.

### c) N-(4-Azepan-1-yl-butyl)-4-pyridin-2-yl-benzamide

Azepane (50 μl, 0.45 mmol) was weighed into a clean glass vial. To this, the crude N-(4-oxo-butyl)-4-pyridin-2-yl-benzamide (100 mg, 0.37 mmol) was added, dissolved in 2 mL of anhydrous dichloromethane. The reaction was left to mix for 90 minutes before addition of sodium triacetoxyborohydride (118 mg, 0.56 mmol), after which it was stirred for 16 hours at room temperature before washing the crude reaction with saturated NaHCO₃ (2 mL solution) and extracting the organic layer. The dichloromethane crude solution was passed through an SCX column, eluting the desired product in 20% ammonia in methanol. Fractions containing the compound were combined and the product purified further using HPLC prep to yield N-(4-Azepan-1-yl-butyl)-4-pyridin-2-yl-benzamide as the formate salt (47 mg, 36% yield).

¹H NMR (CDCl₃) 8.08 (m, 4H), 7.77 (m, 3H), 7.27 (m, 1H), 3.54 (m, 2H), 3.10 (m, 6H), 1.89 (m, 6H), 1.73 (m, 6H)

### EXAMPLE 9

### 5-Piperidin-1-yl-pentanoic acid (3-chloro-phenyl)-amide

Following the general procedure in dichloroethane/dimethylformamide at 55° C., 3-chloroaniline (76 mg, 0.6 mmol) and triethylamine (60 mg, 0.6 mmol) are dissolved in dimethylformamide (0.5 mL) and 5-bromovaleryl chloride (113 mg, 0.57 mmol) in dimethylformamide (0.5 mL) is added dropwise. After 1 h 30 min, piperidine (153 mg, 1.8 mmol) and triethylamine (60 mg, 0.6 mmol) in dimethylformamide (0.5 mL) and the reaction mixture heated at +55° C. for 4 h. Wok-up followed by preparative HPLC affords the title compound (118 mg, 67%) as a white solid as formate salt.

C₁₆H₂₃C₁N₂O Mass (calculated) [294.82]; (found) [M+H+]=295

LC Rt=1.78, 100% (10 min method)

NMR (400 MHz, dmso-d6): 1.48 (2H, m); 1.52 (6H, m); 2.31 (2H, t); 2.48 (6H, m); 7.05 (1H, dd, J=8, 1.2); 7.30 (1H, m); 7.41 (1H, dd, J=8.4, 0.8); 7.80 (1H, s); 8.21 (1H, HCOOH,s); 10.1 (1H, bs).

### EXAMPLE 10

### 5-morpholin-4-yl-pentanoic acid (4-bromo-phenyl)-amide

Prepared according the general procedure in dichloromethane at room temperature to give 6.4 g (93%) of the title compound.

C₁₅H₂₁N₂O₂Br Mass (calculated) [341.24]; found [M+H+]=341/343 (Br)

Lc Rt=2.30, 100%

NMR (400 MHz, DMSO): 1.44 (2H, m); 1.57 (2H, m); 2.29 (8H, m), 3.54 (4H, m), 7.44 (2H, d, J=7 Hz), 7.54 (2H, d, J=7 Hz).

### EXAMPLE 11

### 5-Piperidin-1-yl-pentanoic acid (3-bromo-phenyl)-amide

Prepared according the general procedure in dichloromethane at room temperature to give 1.7 g (33%) of the title compound.

C₁₆H₂₃N₂OBr Mass (calculated) [339.28]; found [M+H+]=339/341 (Br),

Lc Rt=1.86, 98%

NMR (400 MHz, DMSO): 1.51-1.64 (10H, m); 2.34 (2H, m); 2.23 (2H, m); 2.76 (4H, m); 2.97 (2H, m); 7.12-7.264 (2H, m); 7.48 (2H, br d, J=8 Hz); 7.97 (1H, s).

### EXAMPLE 12

### 5-Morpholin-4-yl-pentanoic acid (2′-trifluoromethyl-biphenyl-4-yl)-amide

Prepared according the general procedure in dichloromethane at room temperature followed by Suzuki coupling to give 0.1 g (92%) of the title compound.

C₂₂H₂₅N₂O₂F₃ Mass (calculated) [406.44]; (found) [M+H+]=407

Lc Rt=3.36, 98%

NMR (400 MHz, DMSO): 1.45 (2H, m); 1.6 (2H, m); 2.3 (8H, m); 3.55 (4H, m); 7.21 (2H, d, J=8.4 Hz); 7.36 (1H, d, J=7.3 Hz); 7.56 (1H, m); 7.63 (2H, d, J=8.4 Hz); 7.68 (1H, m); 7.79 (1H, d, J=7.7 Hz)

### EXAMPLE 13

### 4′-[5-(4-Methyl-piperazin-1-yl)-pentanoylamino]-biphenyl-3-carboxylic acid amide

Prepared according the general procedure in dichloromethane at room temperature followed by Suzuki coupling to give 0.07 g (63%) of the title compound.

C₂₃H₃₀N₄O₂Mass (calculated) [394.51]; (found) [M+H+]=395

Lc Rt=1.06, 100%

NMR (400 MHz, DMSO): 1.43 (2H, m); 1.58 (2H, m); 2.10 (3H, s); 2.12-2.44 (12H, m); 7.40 (1H, s); 7.49 (1H, m); 7.68 (4H, m); 7.78 (2H, m); 8.06 (1H, s); 8.11 (1H, s); 9.97 (1H, s).

### EXAMPLE 14

### 5-(4-Acetyl-piperazin-1-yl)-pentanoic acid (2′-methoxy-biphenyl-4-yl)-amide

Prepared according the general procedure in dichloromethane at room temperature followed by Suzuki coupling to give 46 mg (51%) of the title compound.

C₂₄H₃₁N₃O₃ Mass (calculated) [409.53]; (found) [M+H+]=410

LC Rt=2.21, 100% (10 min method)

NMR (400 MHz, CD3OD): 1.62 (2H, m); 1.74(2H, m); 2.07 (3H, s); 2.41-2.49 (8H, m); 3.53 (2H, m); 3.58 (2H, m);3.78 (3H, s); 6.98 (1H, m); 7.04 (1H, d, J=8); 7.27 (2H, m); 7.43 (2H, d, J=8.8); 7.56 (2H, d, J=8.8)

### EXAMPLE 15

### 4-Acetyl-1-[4-(2′,3′-difluoro-biphenyl-4-ylcarbamoyl)-butyl]-[1,4]diazepan-1-ium formate

Prepared according the general procedure in dichloromethane at room temperature followed by Suzuki coupling to give 0.04 g (37%) of the title compound.

C₂₄H₂₉N₃O₂F₂ HCO₂H Mass (calculated) [429.51/46.01]; (found) [M+H+]=430.28

Lc Rt=2.98, 100%

NMR (400 MHz, DMSO): 1.44 (2H, m); 1.58 (2H, m); 1.66 (1H, m); 1.75 (1H, m); 1.96 (3H, s), 2.32 (2H, m); 2.42 (2H, m); 2.52 (3H, m); 2.62 (1H, m); 3.54 (4H, m), 7.24-7.42 (3H, m); 7.5 (2H, d, J=9 Hz); 7.7 (2H, d, J=9 Hz); 8.16 (1H, s); 10.03 (1H, s)

### EXAMPLE 16

### 5-Piperidin-1-yl-pentanoic acid (3′-hydroxy-biphenyl-3-yl)-amide

Prepared according the general procedure in dichloromethane at room temperature followed by Suzuki coupling to give 0.06 g (58%) of the title compound.

C₂₂H₂₈N₂O₂ Mass (calculated) [352.47]; (found) [M+H+]=353.32

Lc Rt=1.90, 99%

NMR (400 MHz, DMSO): 1.34 (2H, m); 1.40-1.47 (6H, m); 1.57 (2H, m); 2.19-2.33 (8H, m); 6.73 (1H, d, J=8 Hz); 6.95 (1H, s); 6.99 (1H, d, J=7 Hz); 7.23 (2H, m); 7.32 (1H, m); 7.51 (1H, d, J=9 Hz); 7.87 (1H, s); 9.56 (1H, br s); 9.94 (1H, s).

### EXAMPLE 17

### 1-(2′-Chloro-biphenyl-4-yl)-3- (4-morpholin-4-yl-butyl)-urea

1-(4-Bromo-phenyl)-3-(4-morpholin-4-yl-butyl)-urea was weighed (0.8 g, 0.22 mmol), placed in 2 necks flask and dissolved in a degassed solution of acetonitrile (4 mL) and water (1 mL). 2-Chloro-phenylboronic acid (0.33 g, 0.24 mmol) and Na2CO3 (0.65 g, 0.6 mmol) and a catalytic amount of Pd[(PPh₃)]₄ werer then added in sequence and the mixture was heated at 80° C. and stirred for 20 hours. The solution was filtered on Celite layer and purified using preparative HPLC.

C₂₁H₂₆C₁N₃O₂ Mass (calculated) [387.91]; (found) [M+H+]=388

Lc Rt: 3.20 (96%)

NMR (400 MHz, MeOH): 1.56-1.58 (2H, m), 1.71 (2H, m), 2.94-2.98 (2H, m), 3.06-3.22 (4H, m), 3.22-3.25 (2H, m), 3.8 (4H, m), 7.24-7.29 (5H, m), 7.37-7.42 (3H, m), 8.31 (1H, s)

### TABLE 1-EXAMPLES 18-254

Table 1 shows a selection of the compounds synthesised, which were prepared according to the method indicated in the last column of the table and discussed in detail in the Experimental Procedures with the synthesis of Examples 1-17. When the compound is indicated as the HCl salt, the salt was formed by dissolution of the free base in methanol and addition of 1 eq 1M HCl in ether followed by evaporation of the solvents. When the compound is indicated as HCOOH (formic acid) salt, the compound was purified by preparative HPLC.

| Example | Structure | Salt | Parent Formula |
|---|---|---|---|
| 8 | 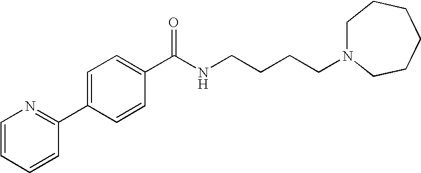 | HCOOH | C22H29N3O |
| 9 | 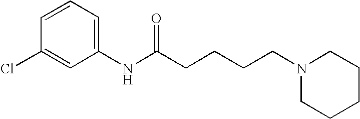 | HCOOH | C16H23N2OCl |
| 10 | 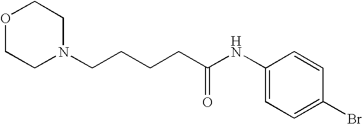 | | C15H21N2O2Br |
| 11 | 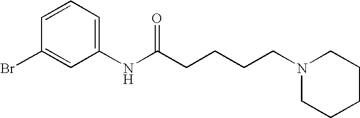 | HCOOH | C16H23N2OBr |
| 12 | 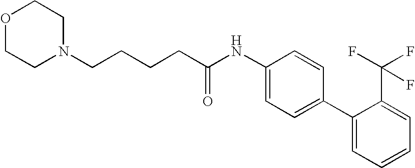 | | C22H25N2O2F3 |
| 13 | 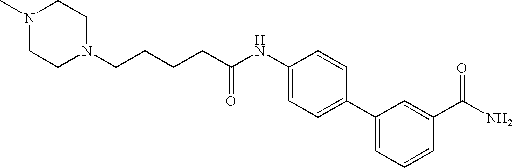 | | C23H30N4O2 |
| 14 | 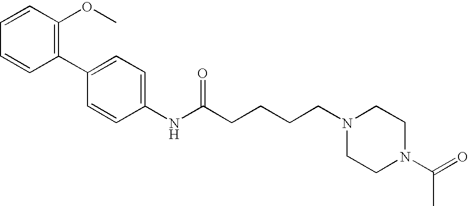 | | C24H31N3O3 |
| 15 | 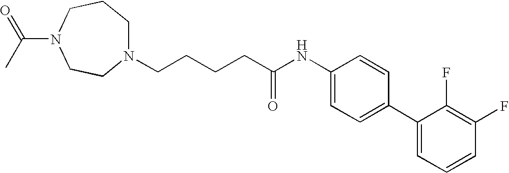 | HCOOH | C24H29N3O2F2 |
| 16 | 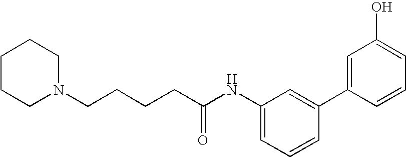 | | C22H28N2O2 |
| 17 | 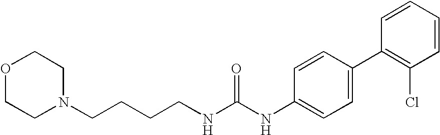 | HCOOH | C21H26N3O2Cl |
| 18 | 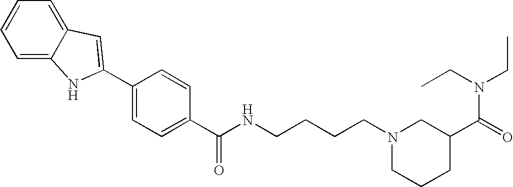 | HCl | C29H38N4O2 |
| 19 | 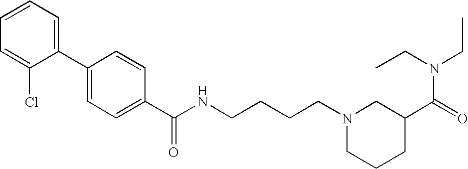 | HCl | C27H36N3O2Cl |
| 20 | 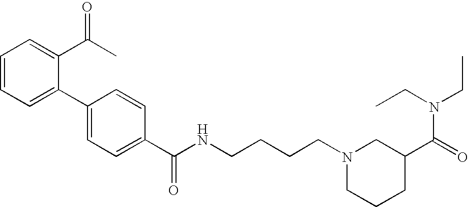 | HCl | C29H39N3O3 |
| 21 | 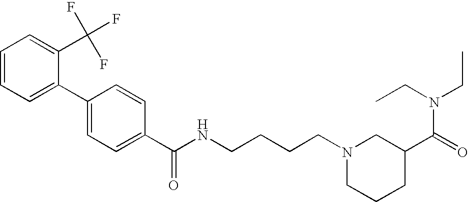 | HCl | C28H36N3O2F3 |
| 22 | 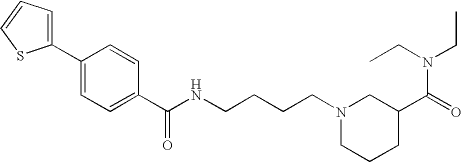 | HCl | C25H35N3O2S |
| 23 | 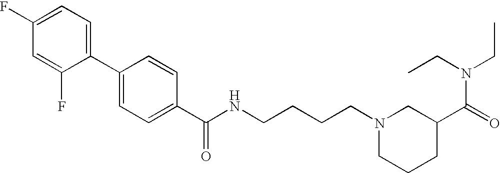 | HCl | C27H35N3O2F2 |
| 24 | 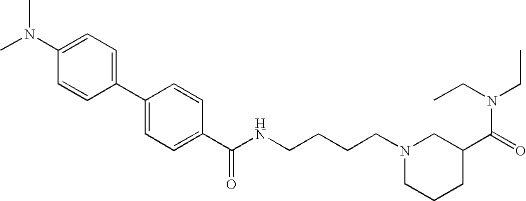 | HCl | C29H42N4O2 |
| 25 | 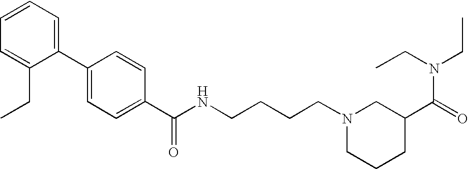 | HCl | C29H41N3O2 |
| 26 | 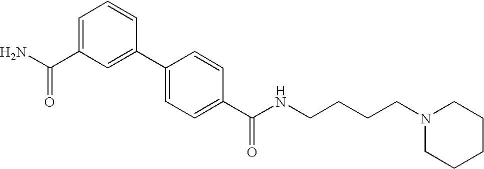 | HCl | C23H29N3O2 |
| 27 | 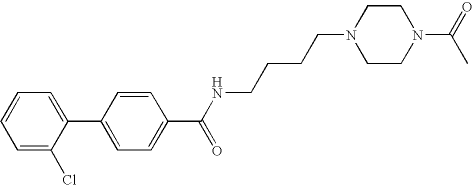 | HCl | C23H28ClN3O2 |
| 28 | 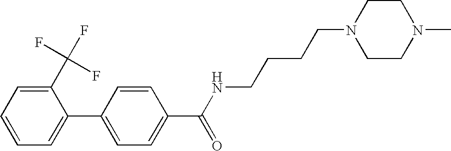 | HCl | C23H28N3OF3 |
| 29 | 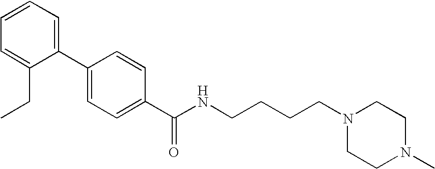 | HCl | C24H33N3O |
| 30 | 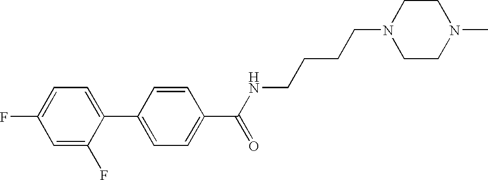 | HCl | C22H27N3OF2 |
| 31 | 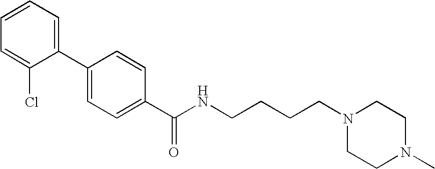 | HCl | C22H28N3OCl |
| 32 | 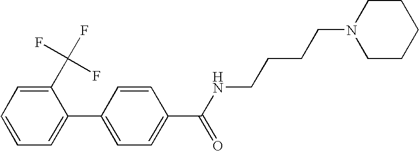 | HCl | C23H27N2OF3 |
| 33 | 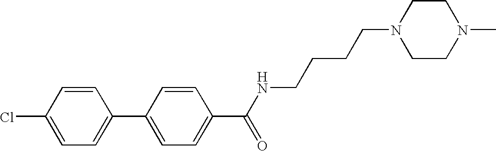 | HCl | C22H28N3OCl |
| 34 | 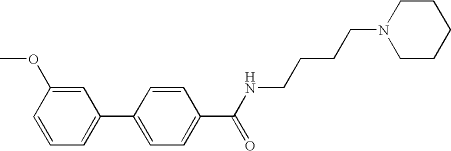 | HCl | C23H30N2O2 |
| 35 | 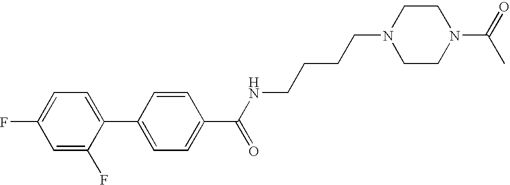 | HCl | C23H27N3O2F2 |
| 36 | 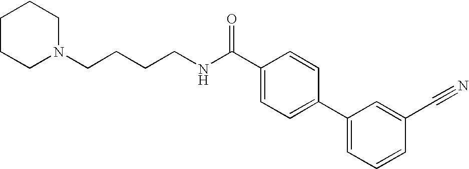 | | C23H27N3O |
| 37 | 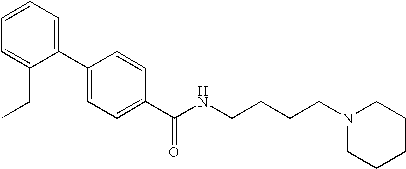 | HCl | C24H32N2O |
| 38 | 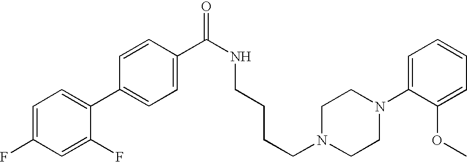 | HCl | C28H31F2N3O2 |
| 39 | 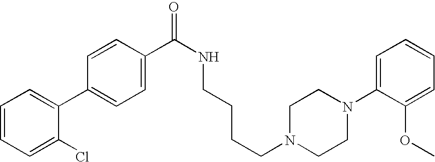 | HCl | C28H32ClN3O2 |
| 40 | 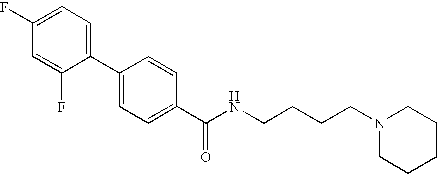 | HCl | C22H26N2OF2 |
| 41 | 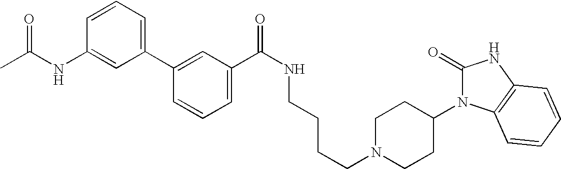 | HCl | C31H35N5O3 |
| 42 | 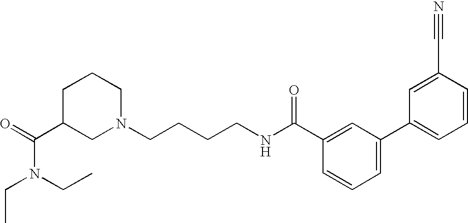 | HCl | C28H36N4O2 |
| 43 | 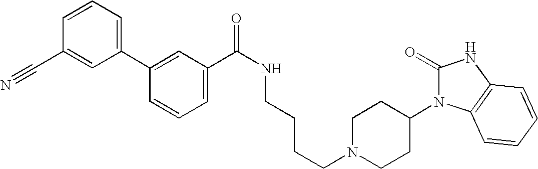 | HCl | C30H31N5O2 |
| 44 | 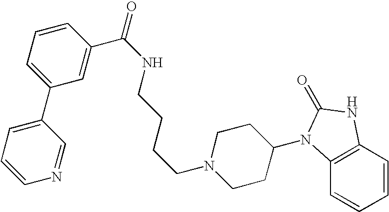 | HCl | C28H31N5O2 |
| 45 | 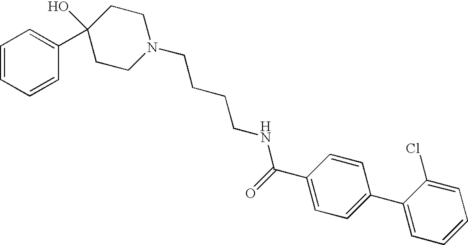 | HCl | C28H31N2O2Cl |
| 46 | 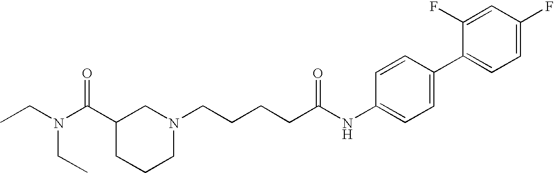 | HCl | C27H35N3O2F2 |
| 47 | 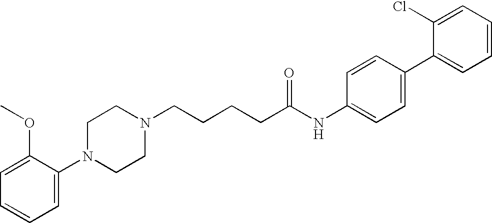 | HCl | C28H32N3O2Cl |
| 48 | 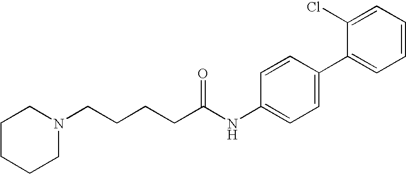 | HCl | C22H27N2OCl |
| 49 | 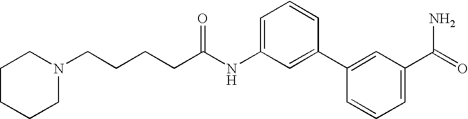 | HCl | C23H29N3O2 |
| 50 | 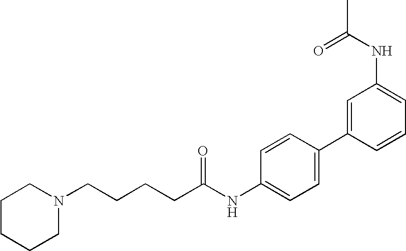 | HCl | C24H31N3O2 |
| 51 | 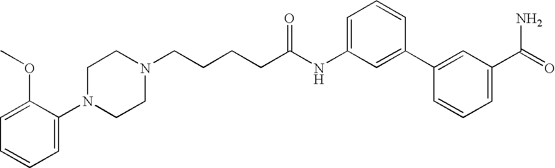 | HCl | C29H34N4O3 |
| 52 | 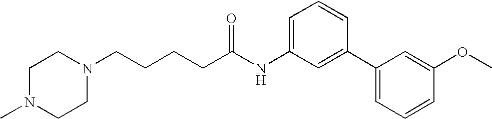 | HCl | C23H31N3O2 |
| 53 | 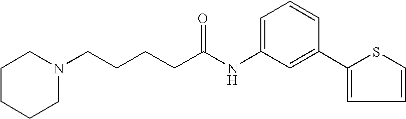 | HCl | C20H26N2OS |
| 54 | 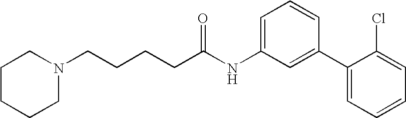 | HCl | C22H27N2OCl |
| 55 | 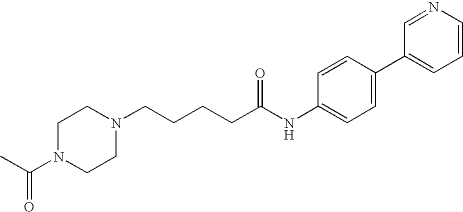 | HCl | C22H28N4O2 |
| 56 | 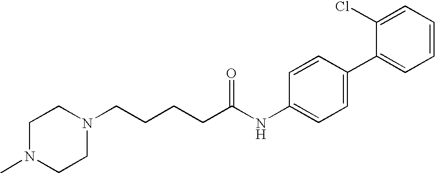 | HCl | C22H28N3OCl |
| 57 | 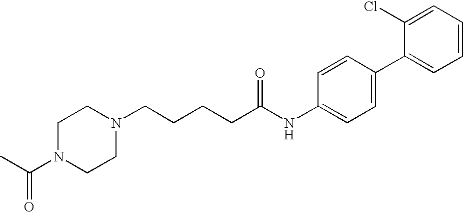 | HCl | C23H28N3O2Cl |
| 58 | 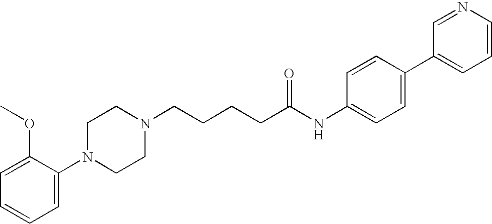 | HCl | C27H32N4O2 |
| 59 | 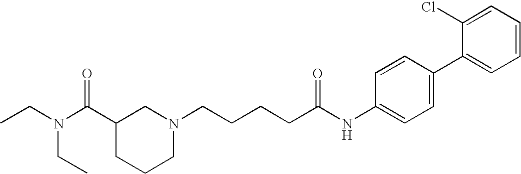 | HCl | C27H36N3O2Cl |
| 60 | 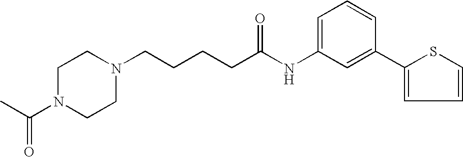 | HCl | C21H27N3O2S |
| 61 | 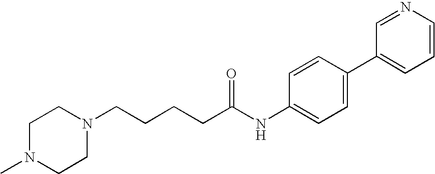 | HCl | C21H28N4O |
| 62 | 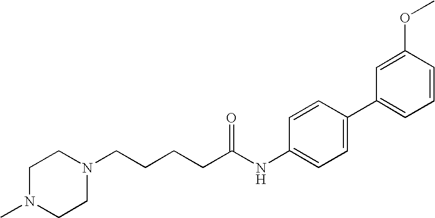 | HCl | C23H31N3O2 |
| 63 | 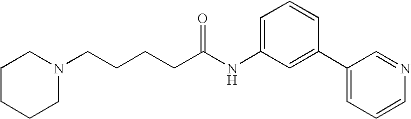 | HCl | C21H27N3O |
| 64 | 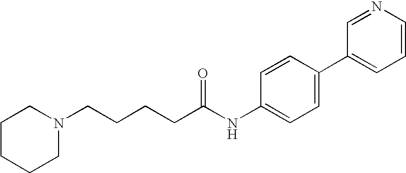 | HCl | C21H27N3O |
| 65 | 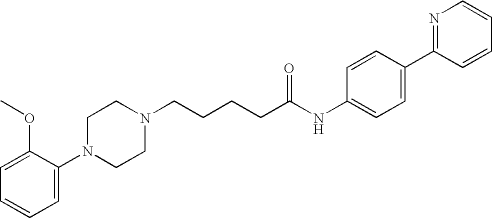 | HCl | C27H32N4O2 |
| 66 | 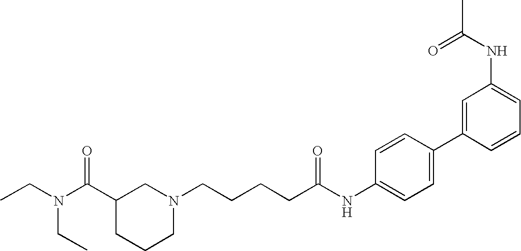 | HCl | C29H40N4O3 |
| 67 | 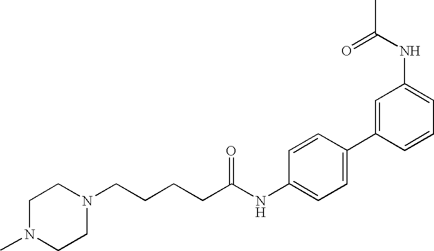 | HCl | C24H32N4O2 |
| 68 | 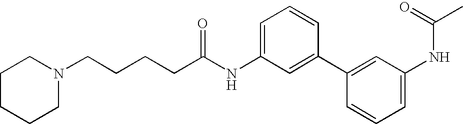 | HCl | C24H31N3O2 |
| 69 | 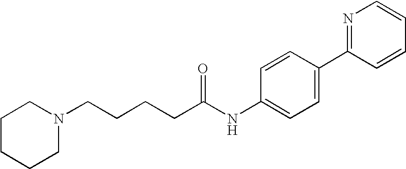 | HCl | C21H27N3O |
| 70 | 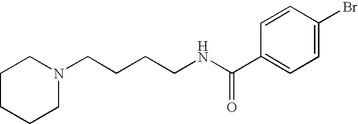 | HCl | C16H23N2OBr |
| 71 | 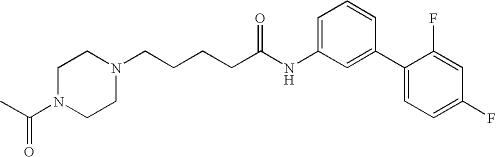 | HCl | C23H27N3O2F2 |
| 72 | 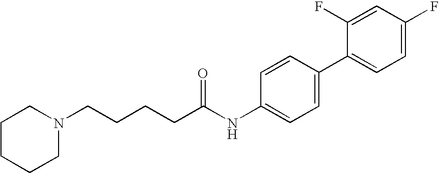 | HCl | C2H26N2OF2 |
| 73 | 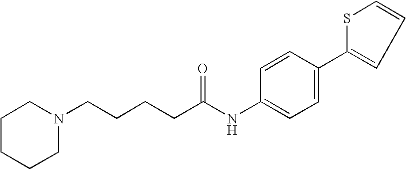 | HCl | C20H26N2OS |
| 74 | 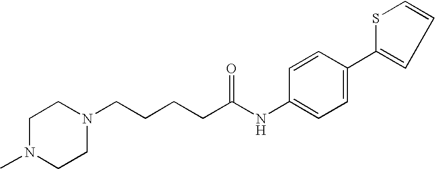 | HCl | C20H27N3OS |
| 75 | 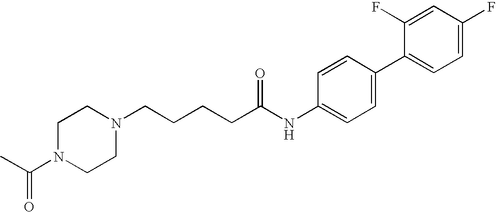 | HCl | C23H27N3O2F2 |
| 76 | 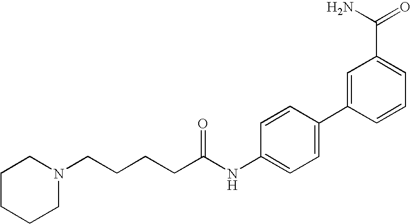 | HCl | C23H29N3O2 |
| 77 | 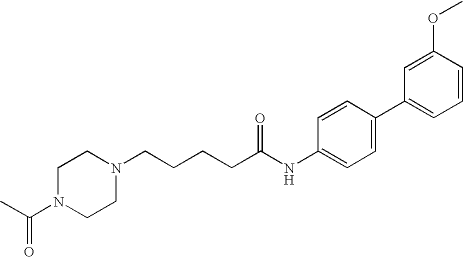 | HCl | C24H31N3O3 |
| 78 | 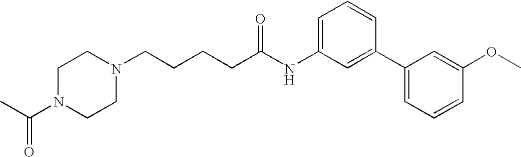 | HCl | C24H31N3O3 |
| 79 | 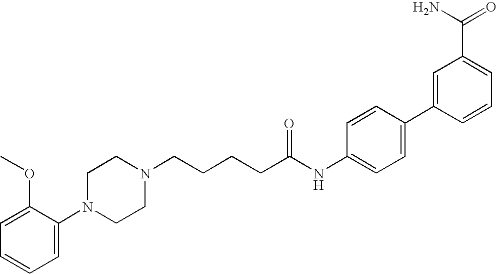 | HCl | C29H34N4O3 |
| 80 | 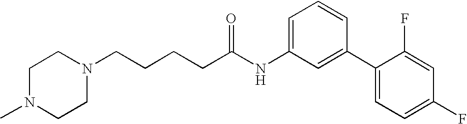 | HCl | C22H27N3OF2 |
| 81 | 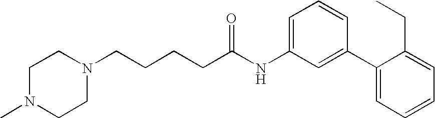 | HCl | C24H33N3O |
| 82 | 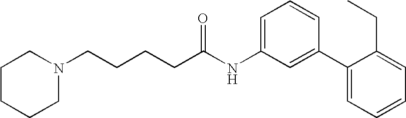 | HCl | C24H32N2O |
| 83 | 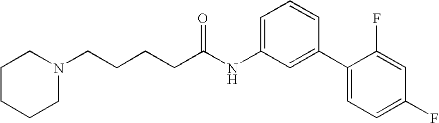 | HCl | C22H26N2OF2 |
| 84 | 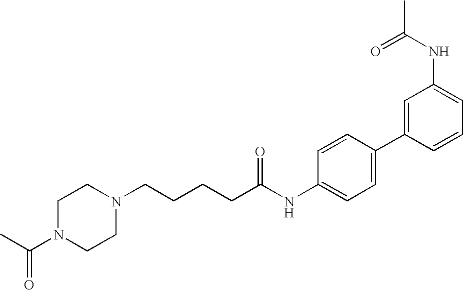 | HCl | C25H32N4O3 |
| 85 | 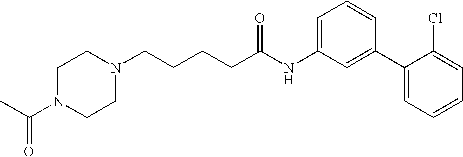 | HCl | C23H28N3O2Cl |
| 86 | 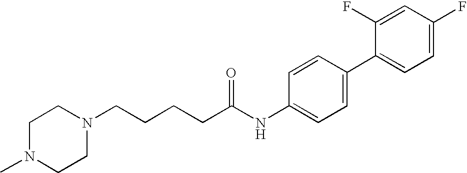 | HCl | C22H27N3OF2 |
| 87 | 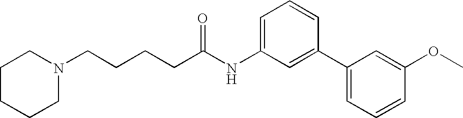 | HCl | C23H30N2O2 |
| 88 | 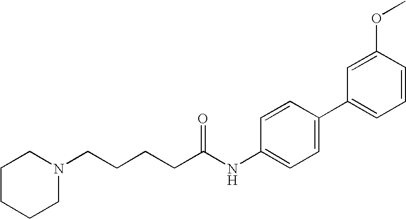 | | C23H30N2O2 |
| 89 | 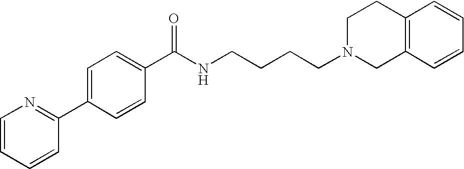 | HCOOH | C25H27N3O |
| 90 | 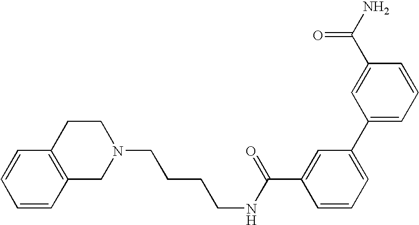 | HCl | C27H29N3O2 |
| 91 | 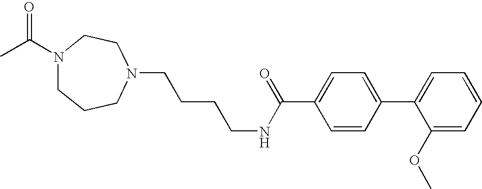 | HCl | C25H33N3O3 |
| 92 | 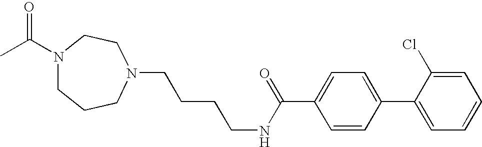 | HCl | C24H30N3O2Cl |
| 93 | 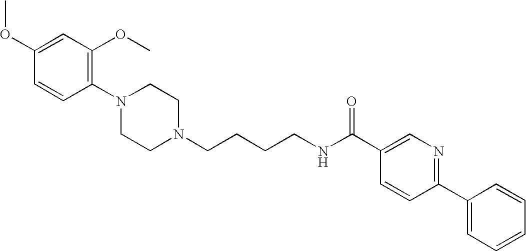 | | C28H34N4O3 |
| 94 | 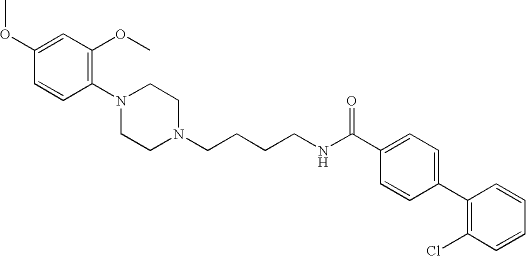 | | C29H34N3O3Cl |
| 95 | 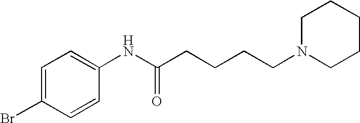 | | C16H23N2OBr |
| 96 | 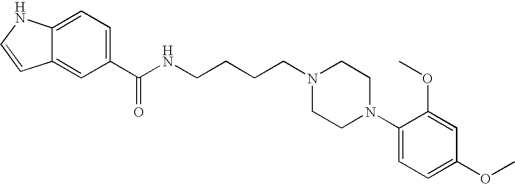 | HCOOH | C25H32N4O3 |
| 97 | 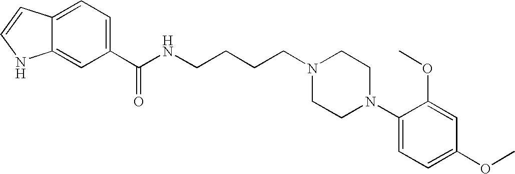 | HCOOH | C25H32N4O3 |
| 98 | 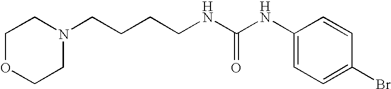 | | C15H22N3O2Br |
| 99 | 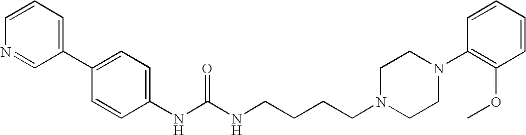 | | C27H33N5O2 |
| 100 | 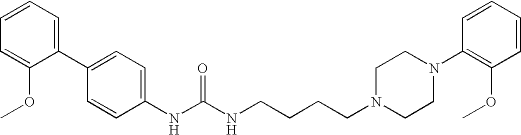 | | C29H36N4O3 |
| 101 | 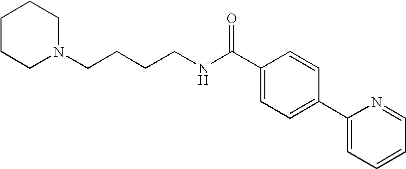 | | C21H27N3O |
| 102 | 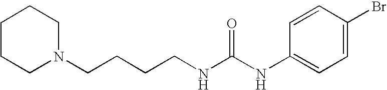 | | C16H24N3OBr |
| 103 | 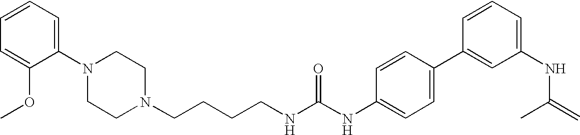 | HCl | C30H37N5O3 |
| 104 | 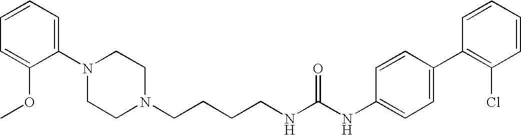 | HCOOH | C28H33N4O2Cl |
| 105 | 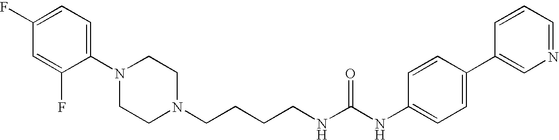 | | C26H29N5OF2 |
| 106 | 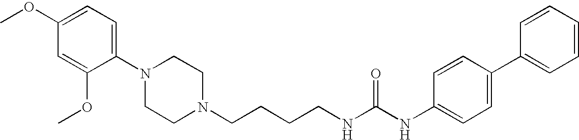 | HCOOH | C28H35N5O3 |
| 107 | 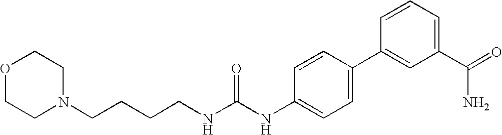 | | C22H28N4O3 |
| 108 | 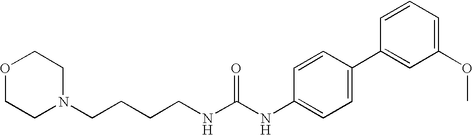 | | C22H29N3O3 |
| 109 | 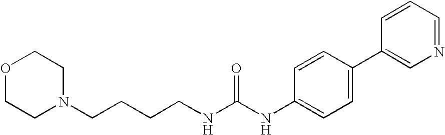 | | C20H26N4O2 |
| 110 | 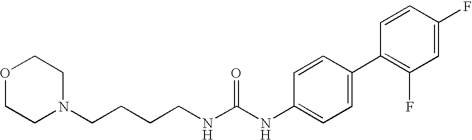 | | C21H25N3O2F2 |
| 111 | 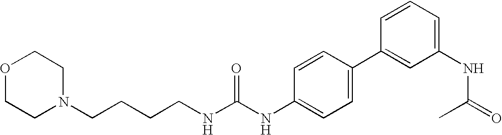 | | C23H30N4O3 |
| 112 | 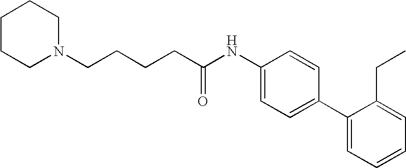 | | C24H32N2O |
| 113 | 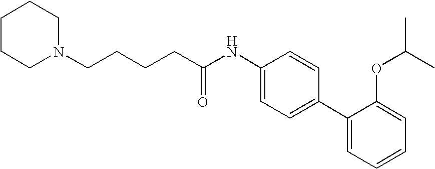 | | C25H34N2O2 |
| 114 | 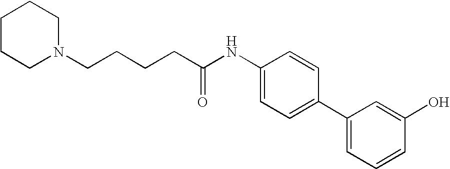 | | C22H28N2O2 |
| 115 | 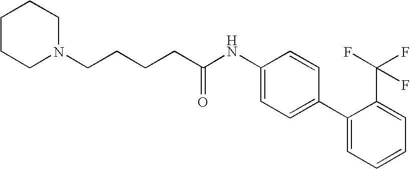 | | C23H27N2OF3 |
| 116 | 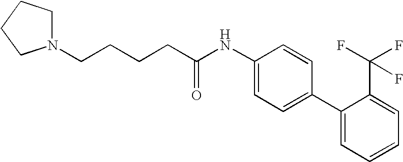 | | C22H25N2OF3 |
| 117 | 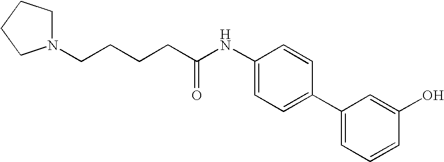 | | C21H26N2O2 |
| 118 | 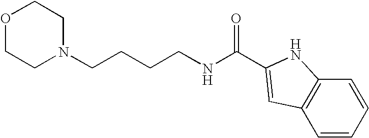 | HCOOH | C17H23N3O2 |
| 119 | 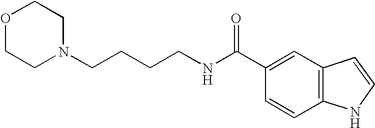 | | C17H23N3O2 |
| 120 | 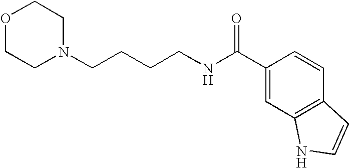 | HCOOH | C17H23N3O2 |
| 121 | 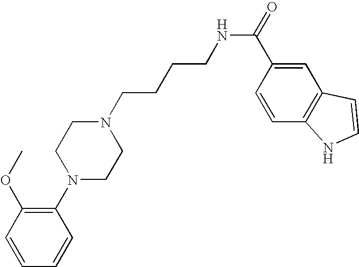 | HCOOH | C24H30N4O2 |
| 122 | 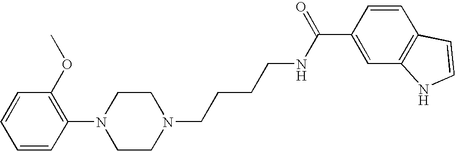 | | C24H30N4O2 |
| 123 | 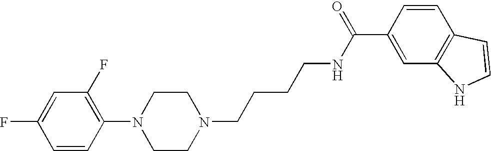 | HCOOH | C23H26N4OF2 |
| 124 | 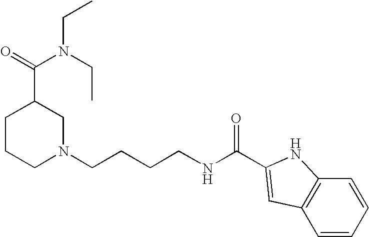 | HCOOH | C23H34N4O2 |
| 125 | 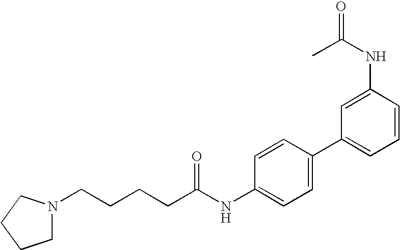 | | C23H29N3O2 |
| 126 | 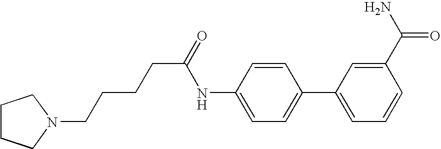 | | C22H27N3O2 |
| 127 | 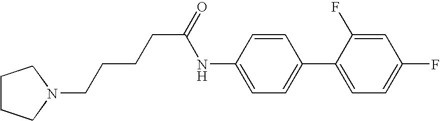 | | C21H24N2OF2 |
| 128 | 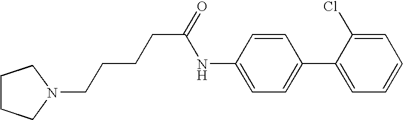 | | C21H25N2OCl |
| 129 | 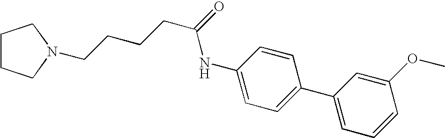 | | C22H28N2O2 |
| 130 | 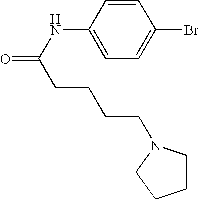 | | C15H21N2OBr |
| 131 | 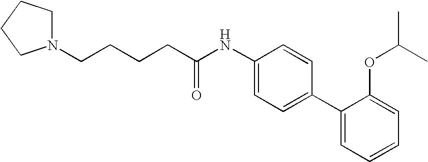 | | C24H32N2O2 |
| 132 | 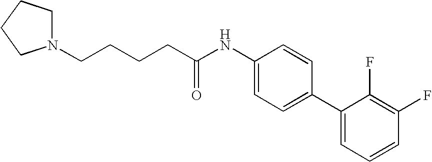 | | C21H24N2OF2 |
| 133 | 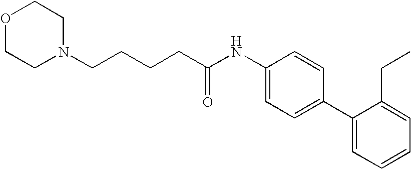 | | C23H30N2O2 |
| 134 | 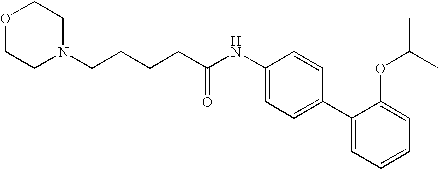 | | C24H32N2O3 |
| 135 | 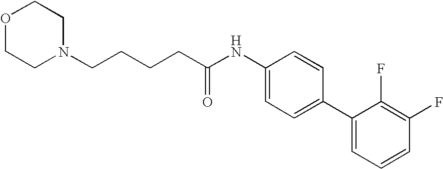 | | C21H24N2O2F2 |
| 136 | 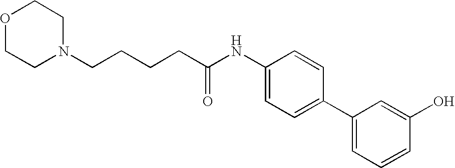 | | C21H26N2O3 |
| 137 | 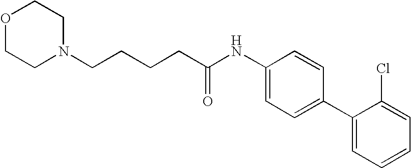 | | C21H25N2O2Cl |
| 138 | 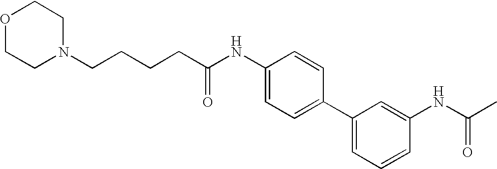 | | C23H29N3O3 |
| 139 | 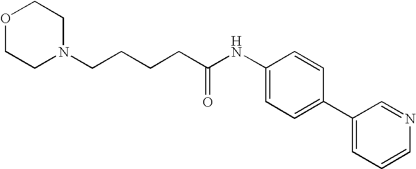 | | C20H25N3O2 |
| 140 | 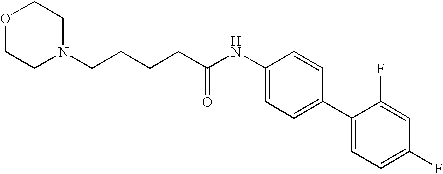 | | C21H24N2O2F2 |
| 141 | 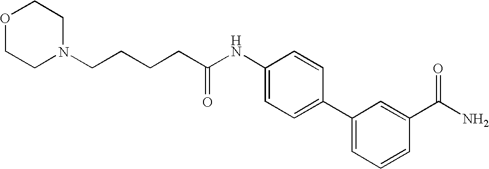 | | C22H27N3O3 |
| 142 | 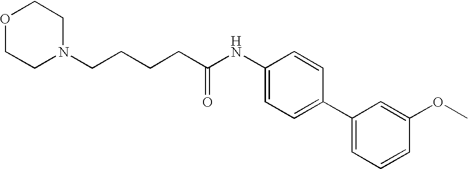 | | C22H28N2O3 |
| 143 | 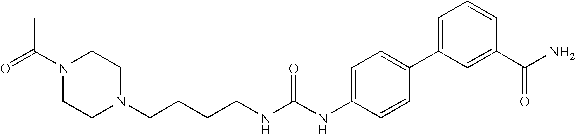 | | C24H31N5O3 |
| 144 | 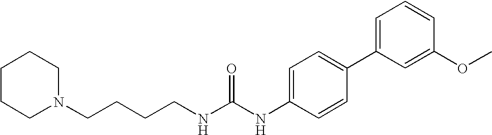 | | C23H31N3O2 |
| 145 | 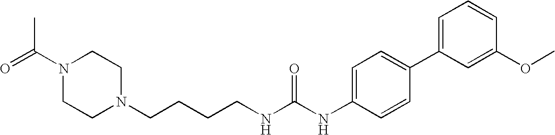 | HCOOH | C24H32N4O3 |
| 146 | 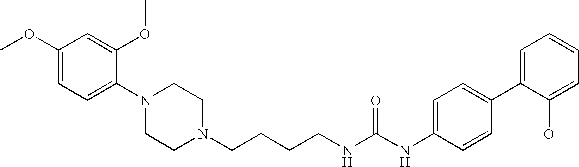 | | C30H38N4O4 |
| 147 | 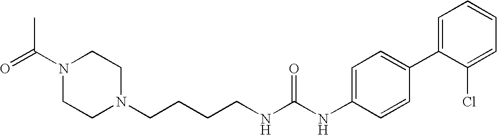 | | C23H29N4O2Cl |
| 148 | 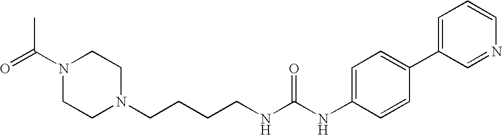 | HCl | C22H29N5O2 |
| 149 | 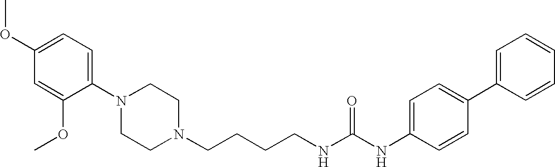 | HCl | C31H39N5O4 |
| 150 | 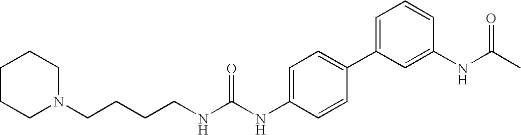 | HCl | C24H32N4O2 |
| 151 | 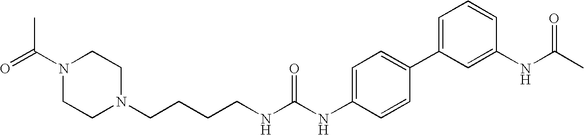 | HCl | C25H33N5O3 |
| 152 | 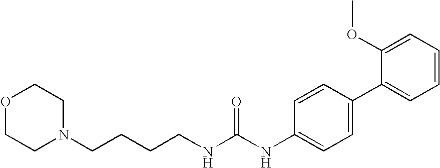 | HCOOH | C22H29N3O3 |
| 153 | 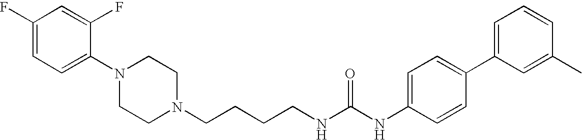 | HCl | C29H33N5O2F2 |
| 154 | 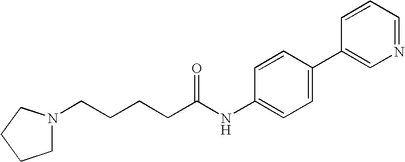 | HCOOH | C20H25N3O |
| 155 | 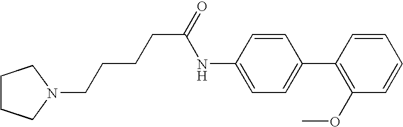 | HCOOH | C22H28N2O2 |
| 156 | 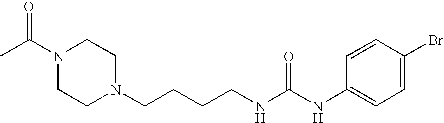 | HCOOH | C17H25N4O2Br |
| 157 | 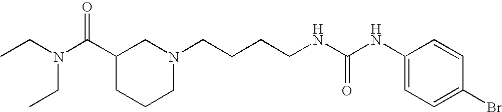 | | C21H33N4O2Br |
| 158 | 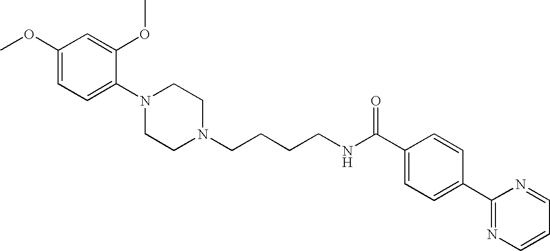 | | C27H33N5O3 |
| 159 | 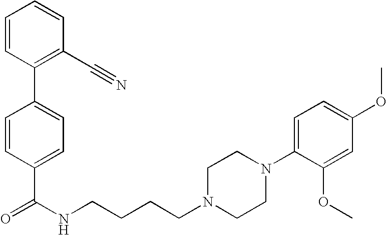 | | C30H34N4O3 |
| 160 | 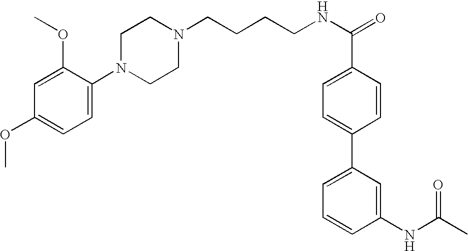 | | C31H38N4O4 |
| 161 | 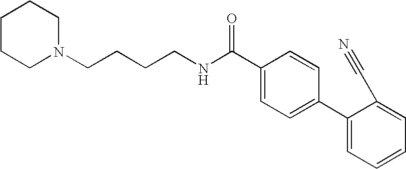 | | C23H27N3O |
| 162 | 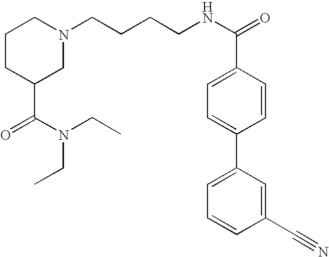 | | C28H36N4O2 |
| 163 | 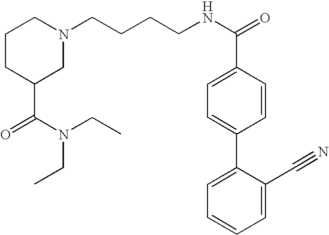 | | C28H36N4O2 |
| 164 | 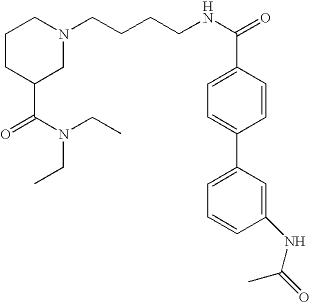 | | C29H40N4O3 |
| 165 | 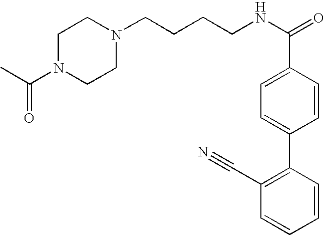 | | C24H28N4O2 |
| 166 | 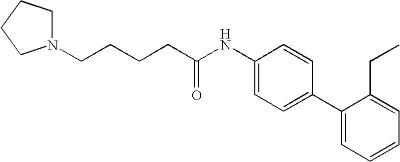 | HCOOH | C23H30N2O |
| 167 | 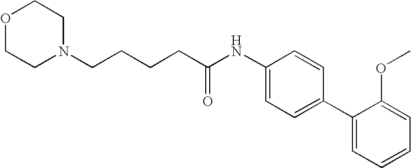 | HCOOH | C22H28N2O3 |
| 168 | 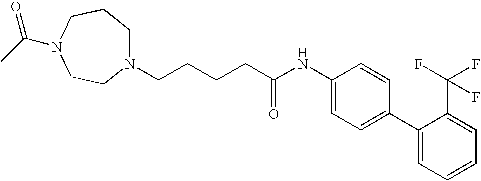 | HCOOH | C25H30N3O2F3 |
| 169 | 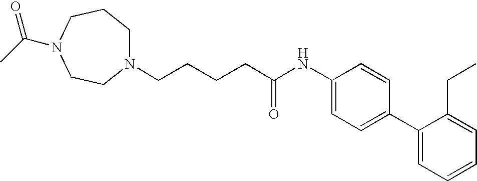 | HCOOH | C26H35N3O2 |
| 170 | 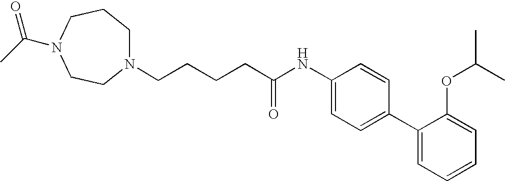 | HCOOH | C27H37N3O3 |
| 171 | 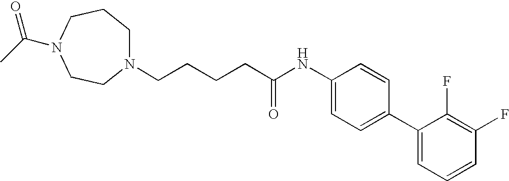 | HCOOH | C24H31N3O3 |
| 172 | 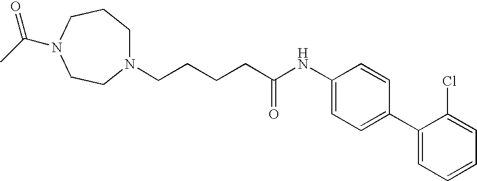 | HCOOH | C24H30N3O2Cl |
| 173 | 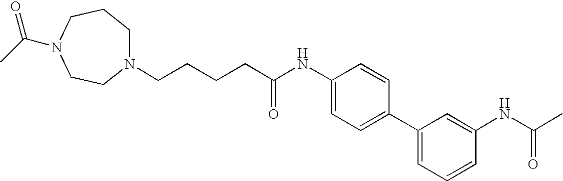 | HCOOH | C26H34N4O3 |
| 174 | 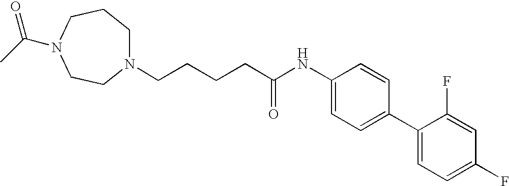 | HCOOH | C24H29N3O2F2 |
| 175 | 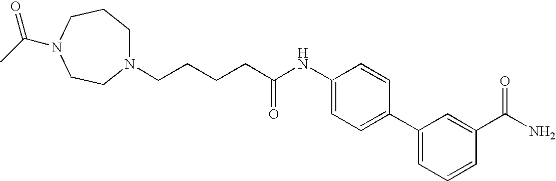 | HCOOH | C25H32N4O3 |
| 176 | 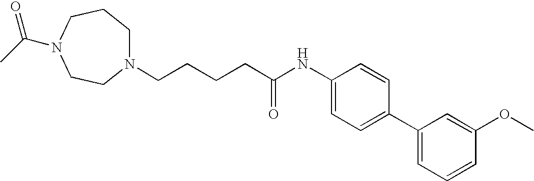 | HCOOH | C25H33N3O3 |
| 177 | 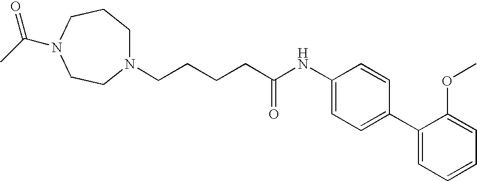 | HCOOH | C25H33N3O3 |
| 178 | 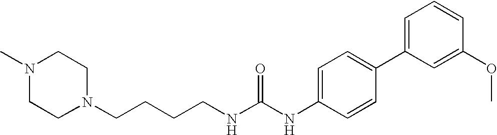 | | C23H32N4O2 |
| 179 | 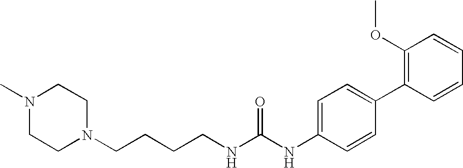 | HCOOH | C23H32N4O2 |
| 180 | 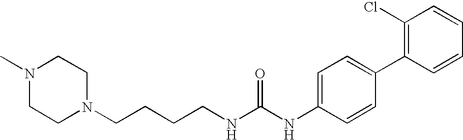 | HCOOH | C22H29N4OCl |
| 181 | 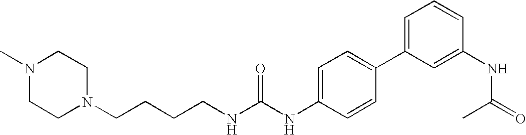 | | C24H33N5O2 |
| 182 | 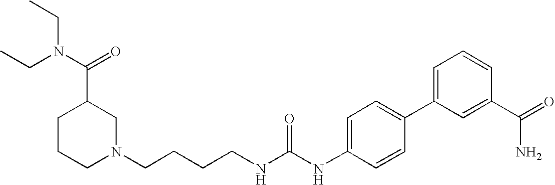 | HCOOH | C28H39N5O3 |
| 183 | 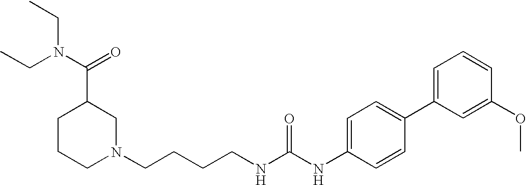 | HCOOH | C28H40N4O3 |
| 184 | 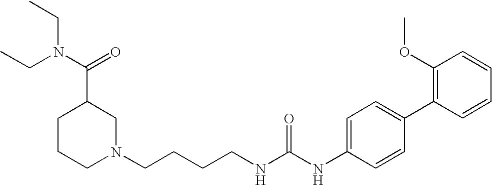 | | C28H40N4O3 |
| 185 | 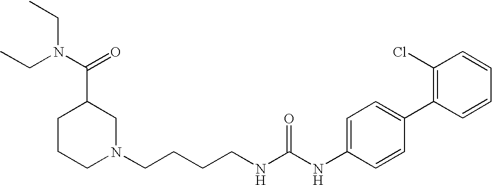 | | C27H37N4O2Cl |
| 186 | 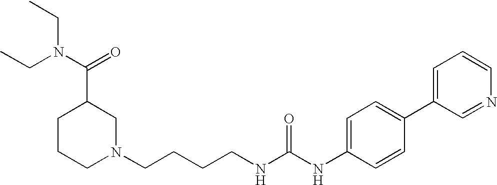 | HCOOH | C26H37N5O2 |
| 187 | 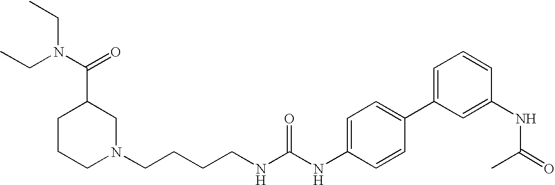 | | C29H41N5O3 |
| 188 | 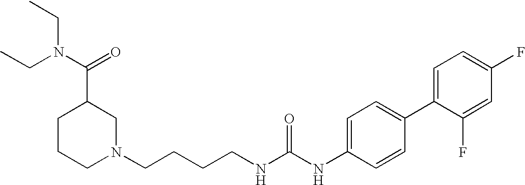 | | C27H36N4O2F2 |
| 189 | 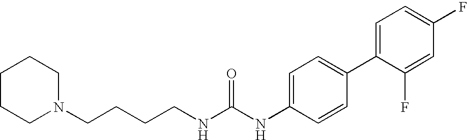 | | C22H27N3OF2 |
| 190 | 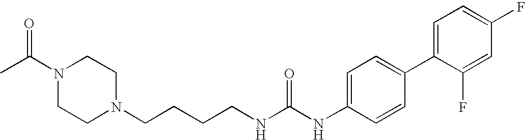 | | C23H28N4O2F2 |
| 191 | 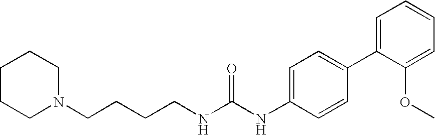 | HCOOH | C23H31N3O2 |
| 192 | 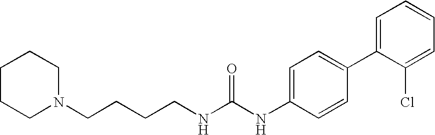 | HCOOH | C22H28N3OCl |
| 193 | 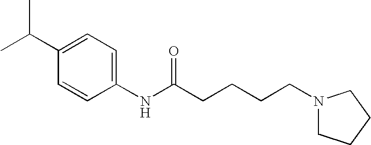 | | C18H28N2O |
| 194 | 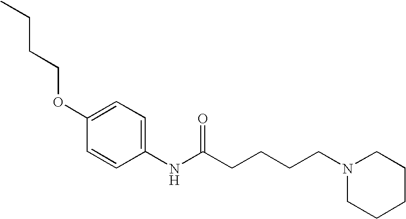 | | C20H32N2O2 |
| 195 | 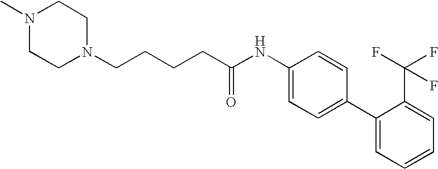 | HCOOH | C23H28N3OF3 |
| 196 | 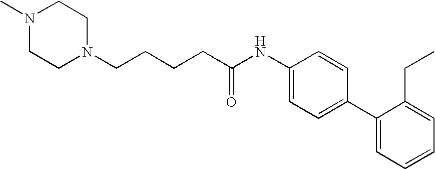 | HCOOH | C24H33N3O |
| 197 | 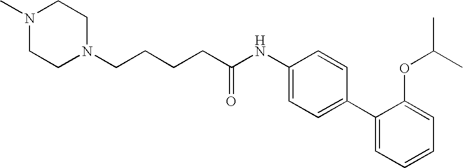 | HCOOH | C25H35N3O2 |
| 198 | 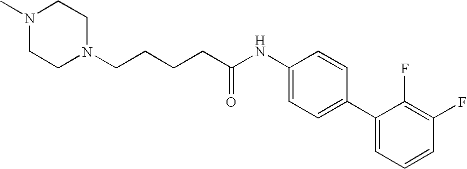 | HCOOH | C22H27N3OF2 |
| 199 | 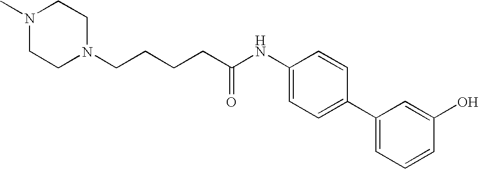 | | C22H29N3O2 |
| 200 | 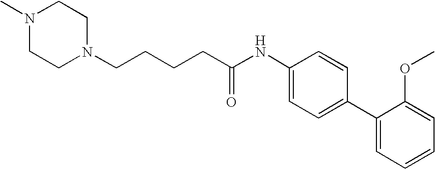 | | C23H31N3O2 |
| 201 | 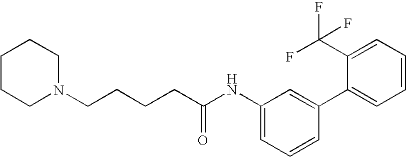 | | C23H27N2OF3 |
| 202 | 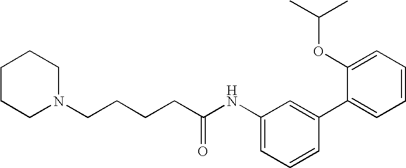 | | C25H34N2O2 |
| 203 | 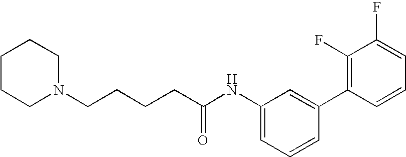 | | C22H26N2OF2 |
| 204 | 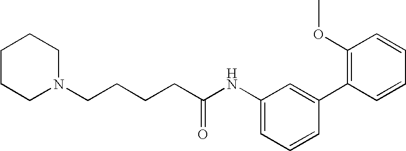 | HCOOH | C23H30N2O2 |
| 205 | 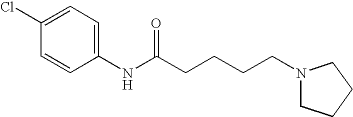 | | C15H21N2OCl |
| 206 | 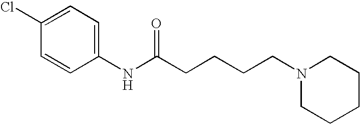 | | C16H23N2OCl |
| 207 | 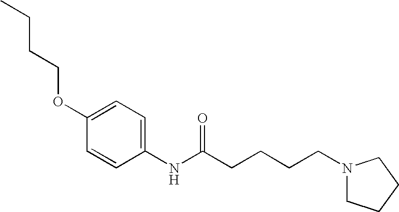 | | C19H30N2O2 |
| 208 | 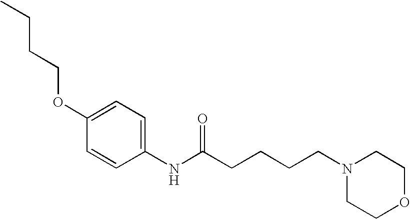 | | C19H30N2O3 |
| 209 | 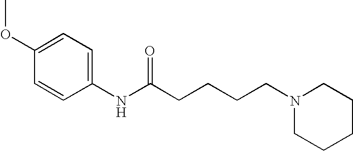 | | C17H26N2O2 |
| 210 | 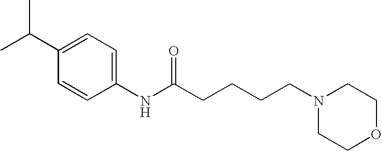 | HCOOH | C18H28N2O2 |
| 211 | 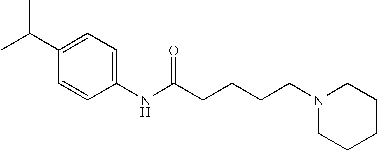 | HCOOH | C19H30N2O |
| 212 | 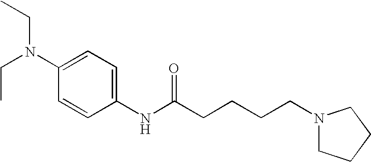 | HCOOH | C19H31N3O |
| 213 | 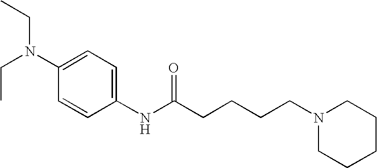 | HCOOH | C20H33N3O |
| 214 | 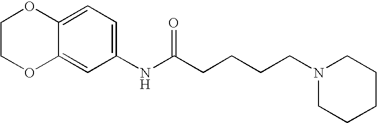 | HOOH | C18H26N2O3 |
| 215 | 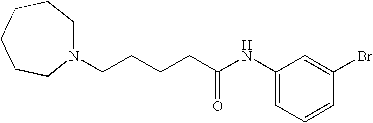 | | C17H25N2OBr |
| 216 | 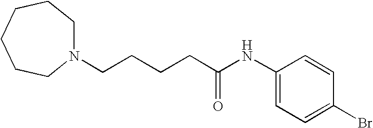 | | C17H25N2OBr |
| 217 | 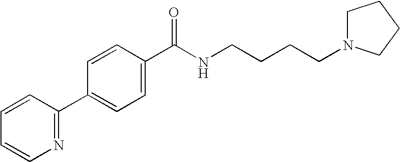 | HCOOH | C20H25N3O |
| 218 | 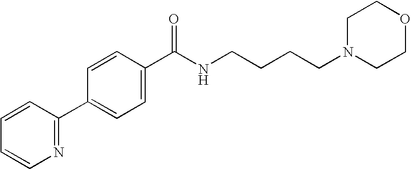 | HCOOH | C20H25N3O2 |
| 219 | 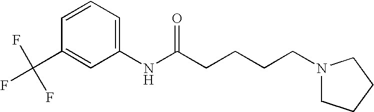 | HCOOH | C16H21N2OF3 |
| 220 | 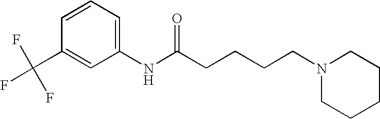 | HCOOH | C17H23N2OF3 |
| 221 | 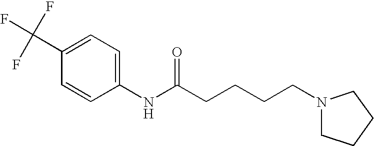 | HCOOH | C16H21N2OF3 |
| 222 | 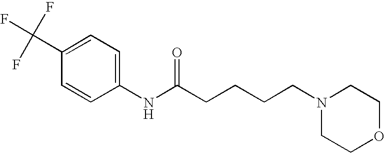 | HCOOH | C16H21N2O2F3 |
| 223 | 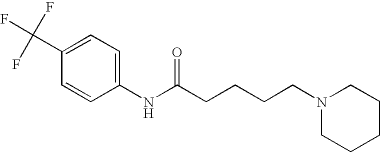 | HCOOH | C17H23N2OF3 |
| 224 | 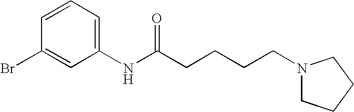 | HCOOH | C15H21N2OBr |
| 225 | 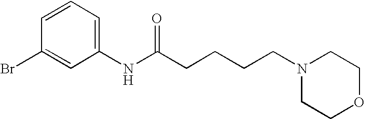 | HCOOH | C15H21N2O2Br |
| 226 | 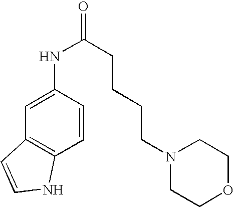 | HCOOH | C17H23N3O2 |
| 227 | 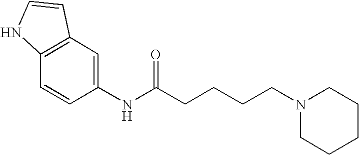 | HCOOH | C18H25N3O |
| 228 | 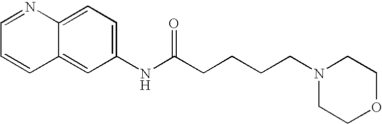 | HCOOH | C18H23N3O2 |
| 229 | 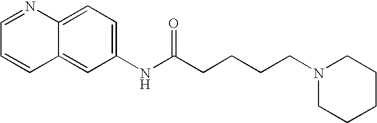 | HCOOH | C19H25N3O |
| 230 | 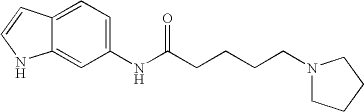 | HCOOH | C17H23N3O |
| 231 | 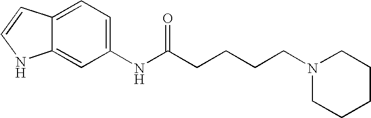 | HCOOH | C18H25N3O |
| 232 | 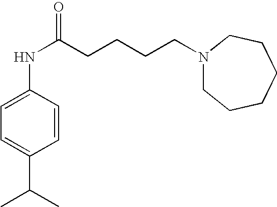 | HCOOH | C20H32N2O |
| 233 | 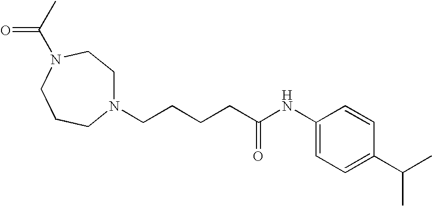 | HCOOH | C21H33N3O2 |
| 234 | 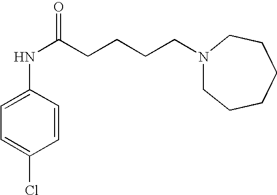 | HCOOH | C17H25N2OCl |
| 235 | 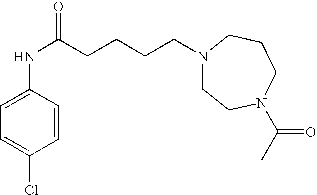 | HCOOH | C18H25N3O2Cl |
| 236 | 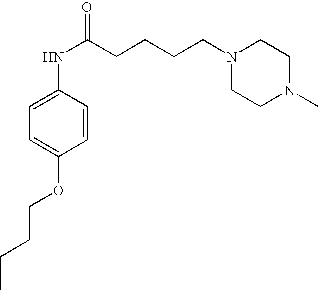 | HCOOH | C20H33N3O2 |
| 237 | 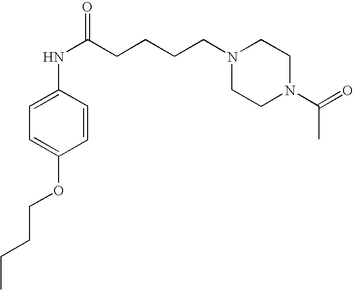 | HCOOH | C21H33N3O3 |
| 238 | 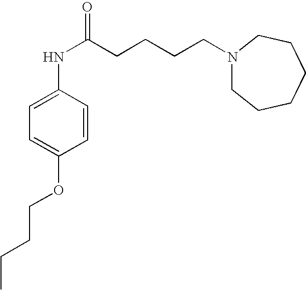 | HCOOH | C21H34N2O2 |
| 239 | 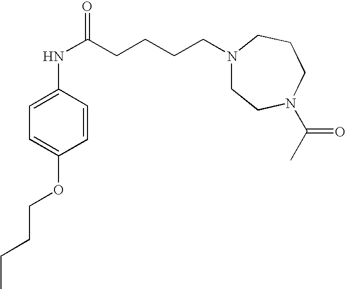 | HCOOH | C21H34N4O2 |
| 240 | 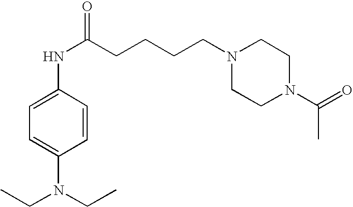 | HCOOH | C21H34N4O2 |
| 241 | 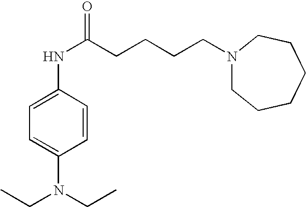 | HCOOH | C21H35N3O |
| 242 | 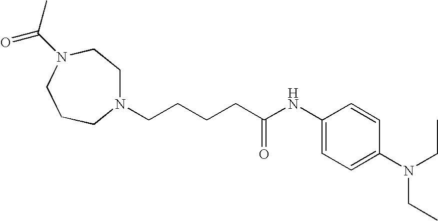 | HCOOH | C22H36N4O2 |
| 243 | 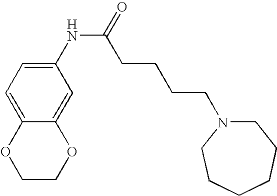 | HCOOH | C19H28N2O3 |
| 244 | 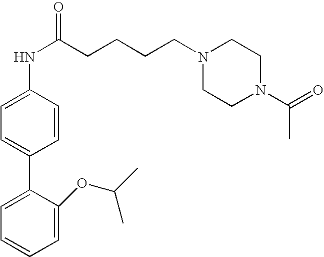 | | C26H35N3O3 |
| 245 | 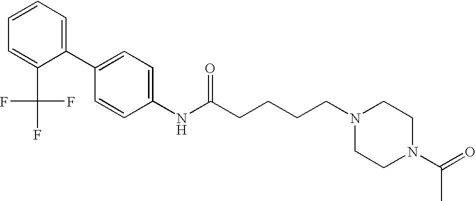 | | C24H28N3O2F3 |
| 246 | 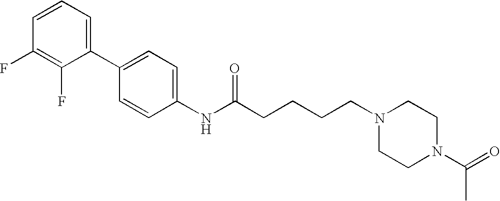 | | C23H27N3O2F2 |
| 247 | 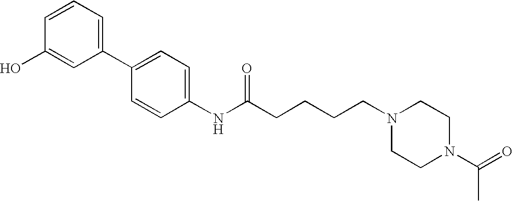 | | C23H29N3O3 |
| 248 | 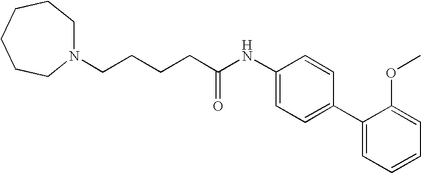 | HCOOH | C24H32N2O2 |
| 249 | 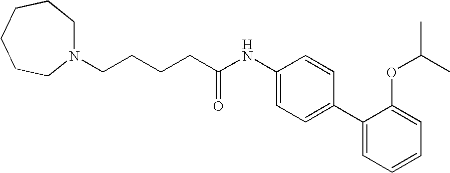 | HCOOH | C26H36N2O2 |
| 250 | 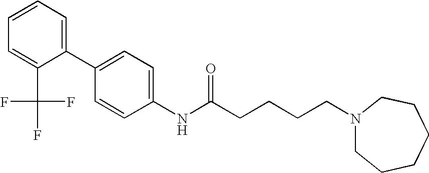 | HCOOH | C24H29N2OF3 |
| 251 | 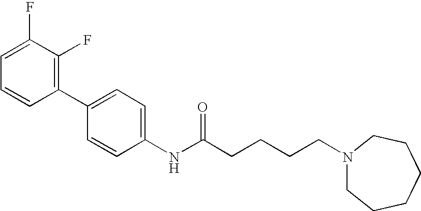 | HCOOH | C23H28N2OF2 |
| 252 | 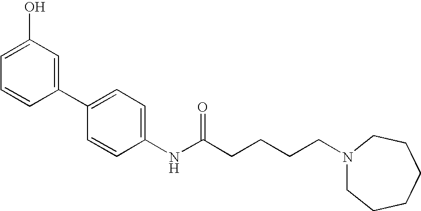 | | C23H30N2O2 |
| 253 | 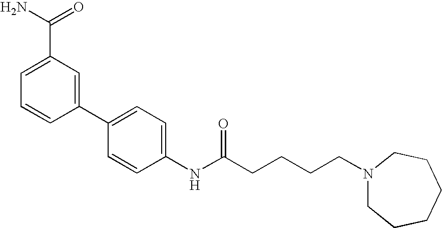 | HCOOH | C24H31N3O2 |
| 254 | 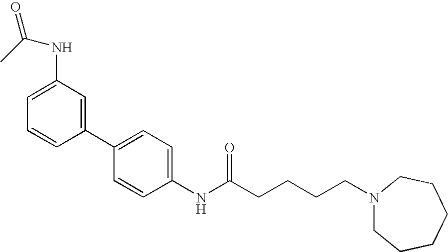 | HCOOH | C25H33N3O2 |

| | | | | | LC | |
|---|---|---|---|---|---|---|
| | Parent | | LC purity | | method | |
| Example | MW | Mass found | % | LC Rt | (min) | Synthetic Method |
| 8 | 351.49 | 352 | 100 | 1.79 | 10 | acid-amine coupling |
| | | | | | | with CDI, room temp. |
| 9 | 294.82 | 295 | 100 | 1.78 | 10 | from 5-bromopentanoyl |
| | | | | | | chloride in DCM/DMF, |
| | | | | | | 55 C. |
| 10 | 341.24 | 341, 343 | 100 | 2.3 | 10 | from 5-bromopentanoyl |
| | | | | | | chloride, 0 0 C.-rt |
| 11 | 339.27 | 339, 341 | 100 | 1.96 | 10 | from 5-bromopentanoyl |
| | | | | | | chloride in DCM/DMF, |
| | | | | | | 55 C. |
| 12 | 406.44 | 407 | 98 | 3.36 | 10 | from 5-bromopentanoyl |
| | | | | | | chloride 0 C.-rt, followed |
| | | | | | | by Suzuki coupling |
| 13 | 394.51 | 395 | 100 | 1.06 | 10 | from 5-bromopentanoyl |
| | | | | | | chloride 0 C.-rt, followed |
| | | | | | | by Suzuki coupling |
| 14 | 409.52 | 410 | 100 | 2.21 | 10 | from 5-bromopentanoyl |
| | | | | | | chloride 0 C.-rt, followed |
| | | | | | | by Suzuki coupling |
| 15 | 429.50 | 430 | 100 | 2.98 | 10 | from 5-bromopentanoyl |
| | | | | | | chloride 0 C.-rt, followed |
| | | | | | | by Suzuki coupling |
| 16 | 352.47 | 353 | 99 | 1.9 | 10 | from 5-bromopentanoyl |
| | | | | | | chloride 0 C.-rt, followed |
| | | | | | | by Suzuki coupling |
| 17 | 387.90 | 388 | 98 | 3.2 | 10 | urea synthesis followed |
| | | | | | | by Suzuki under thermal |
| | | | | | | heating |
| 18 | 474.64 | 475 | 98 | 1.35 | 2.5 | reductive alkylation on |
| | | | | | | N-(4-oxo- |
| | | | | | | butyl)bromobenzamide |
| | | | | | | followed by Suzuki |
| | | | | | | coupling |
| 19 | 470.05 | 470 | 97 | 1.34 | 2.5 | reductive alkylation on |
| | | | | | | N-(4-oxo- |
| | | | | | | butyl)bromobenzamide |
| | | | | | | followed by Suzuki |
| | | | | | | coupling |
| 20 | 477.64 | 478 | 94 | 1.18 | 2.5 | reductive alkylation on |
| | | | | | | N-(4-oxo- |
| | | | | | | butyl)bromobenzamide |
| | | | | | | followed by Suzuki |
| | | | | | | coupling |
| 21 | 503.60 | 504 | 99 | 1.42 | 2.5 | reductive alkylation on |
| | | | | | | N-(4-oxo- |
| | | | | | | butyl)bromobenzamide |
| | | | | | | followed by Suzuki |
| | | | | | | coupling |
| 22 | 441.63 | 442 | 100 | 1.29 | 2.5 | reductive alkylation on |
| | | | | | | N-(4-oxo- |
| | | | | | | butyl)bromobenzamide |
| | | | | | | followed by Suzuki |
| | | | | | | coupling |
| 23 | 471.58 | 472 | 98 | 1.37 | 2.5 | reductive alkylation on |
| | | | | | | N-(4-oxo- |
| | | | | | | butyl)bromobenzamide |
| | | | | | | followed by Suzuki |
| | | | | | | coupling |
| 24 | 478.56 | 479 | 96 | 1.37 | 2.5 | reductive alkylation on |
| | | | | | | N-(4-oxo- |
| | | | | | | butyl)bromobenzamide |
| | | | | | | followed by Suzuki |
| | | | | | | coupling |
| 25 | 463.65 | 464 | 100 | 1.46 | 2.5 | reductive alkylation on |
| | | | | | | N-(4-oxo- |
| | | | | | | butyl)bromobenzamide |
| | | | | | | followed by Suzuki |
| | | | | | | coupling |
| 26 | 379.50 | 380 | 100 | 1.12 | 2.5 | reductive alkylation on |
| | | | | | | N-(4-oxo- |
| | | | | | | butyl)bromobenzamide |
| | | | | | | followed by Suzuki |
| | | | | | | coupling |
| 27 | 414.00 | 414, 416 | 100 | 1.17 | 2.5 | reductive alkylation on |
| | | | | | | N-(4-oxo- |
| | | | | | | butyl)bromobenzamide |
| | | | | | | followed by Suzuki |
| | | | | | | coupling |
| 28 | 419.48 | 420 | 100 | 1.22 | 2.5 | reductive alkylation on |
| | | | | | | N-(4-oxo- |
| | | | | | | butyl)bromobenzamide |
| | | | | | | followed by Suzuki |
| | | | | | | coupling |
| 29 | 379.54 | 380 | 100 | 1.29 | 2.5 | reductive alkylation on |
| | | | | | | N-(4-oxo- |
| | | | | | | butyl)bromobenzamide |
| | | | | | | followed by Suzuki |
| | | | | | | coupling |
| 30 | 387.47 | 388 | 100 | 1.16 | 2.5 | reductive alkylation on |
| | | | | | | N-(4-oxo- |
| | | | | | | butyl)bromobenzamide |
| | | | | | | followed by Suzuki |
| | | | | | | coupling |
| 31 | 385.93 | 386 | 100 | 1.2 | 2.5 | reductive alkylation on |
| | | | | | | N-(4-oxo- |
| | | | | | | butyl)bromobenzamide |
| | | | | | | followed by Suzuki |
| | | | | | | coupling |
| 32 | 404.47 | 405 | 92 | 1.65 | 2.5 | reductive alkylation on |
| | | | | | | N-(4-oxo- |
| | | | | | | butyl)bromobenzamide |
| | | | | | | followed by Suzuki |
| | | | | | | coupling |
| 33 | 385.93 | 386 | 100 | 1.25 | 2.5 | reductive alkylation on |
| | | | | | | N-(4-oxo- |
| | | | | | | butyl)bromobenzamide |
| | | | | | | followed by Suzuki |
| | | | | | | coupling |
| 34 | 366.50 | 367 | 100 | 1.55 | 2.5 | reductive alkylation on |
| | | | | | | N-(4-oxo- |
| | | | | | | butyl)bromobenzamide |
| | | | | | | followed by Suzuki |
| | | | | | | coupling |
| 35 | 415.48 | 416 | 100 | 1.15 | 2.5 | reductive alkylation on |
| | | | | | | N-(4-oxo- |
| | | | | | | butyl)bromobenzamide |
| | | | | | | followed by Suzuki |
| | | | | | | coupling |
| 36 | 361.48 | 362 | 100 | 2.69 | 10 | acid-amine coupling |
| | | | | | | with CDI, room temp. |
| 37 | 364.52 | 365 | 100 | 1.75 | 2.5 | reductive alkylation on |
| | | | | | | N-(4-oxo- |
| | | | | | | butyl)bromobenzamide |
| | | | | | | followed by Suzuki |
| | | | | | | coupling |
| 38 | 479.60 | 480 | 97 | 1.55 | 2.5 | reductive alkylation on |
| | | | | | | N-(4-oxo- |
| | | | | | | butyl)bromobenzamide |
| | | | | | | followed by Suzuki |
| | | | | | | coupling |
| 39 | 478.00 | 478, 480 | 95 | 1.55 | 2.5 | reductive alkylation on |
| | | | | | | N-(4-oxo- |
| | | | | | | butyl)bromobenzamide |
| | | | | | | followed by Suzuki |
| | | | | | | coupling |
| 40 | 372.45 | 373 | 98 | 1.63 | 2.5 | reductive alkylation on |
| | | | | | | N-(4-oxo- |
| | | | | | | butyl)bromobenzamide |
| | | | | | | followed by Suzuki |
| | | | | | | coupling |
| 41 | 525.64 | 526 | 100 | 1.16 | 2.5 | reductive alkylation on |
| | | | | | | N-(4-oxo- |
| | | | | | | butyl)bromobenzamide |
| | | | | | | followed by Suzuki |
| | | | | | | coupling |
| 42 | 460.61 | 461 | 100 | 1.24 | 2.5 | reductive alkylation on |
| | | | | | | N-(4-oxo- |
| | | | | | | butyl)bromobenzamide |
| | | | | | | followed by Suzuki |
| | | | | | | coupling |
| 43 | 493.60 | 494 | 100 | 1.33 | 2.5 | reductive alkylation on |
| | | | | | | N-(4-oxo- |
| | | | | | | butyl)bromobenzamide |
| | | | | | | followed by Suzuki |
| | | | | | | coupling |
| 44 | 469.58 | 470 | 100 | 1.09 | 2.5 | reductive alkylation on |
| | | | | | | N-(4-oxo- |
| | | | | | | butyl)bromobenzamide |
| | | | | | | followed by Suzuki |
| | | | | | | coupling |
| 45 | 463.01 | 462, 464 | 91 | 1.1 | 2.5 | reductive alkylation on |
| | | | | | | N-(4-oxo- |
| | | | | | | butyl)bromobenzamide |
| | | | | | | followed by Suzuki |
| | | | | | | coupling |
| 46 | 472.58 | 472 | 91 | 1.45 | 2.5 | from 5-bromopentanoyl |
| | | | | | | chloride 0 C.-rt, followed |
| | | | | | | by Suzuki coupling |
| 47 | 478.03 | 478 | 98 | 1.63 | 2.5 | from 5-bromopentanoyl |
| | | | | | | chloride 0 C.-rt, followed |
| | | | | | | by Suzuki coupling |
| 48 | 370.92 | 370, 372 | 100 | 1.8 | 2.5 | from 5-bromopentanoyl |
| | | | | | | chloride 0 C.-rt, followed |
| | | | | | | by Suzuki coupling |
| 49 | 379.50 | 380 | 100 | 1.21 | 2.5 | from 5-bromopentanoyl |
| | | | | | | chloride 0 C.-rt, followed |
| | | | | | | by Suzuki coupling |
| 50 | 393.52 | 394 | 92 | 2.63 | 10 | from 5-bromopentanoyl |
| | | | | | | chloride 0 C.-rt, followed |
| | | | | | | by Suzuki coupling |
| 51 | 486.61 | 487 | 96 | 1.23 | 2.5 | from 5-bromopentanoyl |
| | | | | | | chloride 0 C.-rt, followed |
| | | | | | | by Suzuki coupling |
| 52 | 381.51 | 382 | 100 | 1.25 | 2.5 | from 5-bromopentanoyl |
| | | | | | | chloride 0 C.-rt, followed |
| | | | | | | by Suzuki coupling |
| 53 | 342.50 | 343 | 100 | 1.68 | 2.5 | from 5-bromopentanoyl |
| | | | | | | chloride 0 C.-rt, followed |
| | | | | | | by Suzuki coupling |
| 54 | 370.92 | 370, 372 | 100 | 1.79 | 2.5 | from 5-bromopentanoyl |
| | | | | | | chloride 0 C.-rt, followed |
| | | | | | | by Suzuki coupling |
| 55 | 380.48 | 381 | 93 | 0.87 | 2.5 | from 5-bromopentanoyl |
| | | | | | | chloride 0 C.-rt, followed |
| | | | | | | by Suzuki coupling |
| 56 | 385.93 | 385, 387 | 100 | 1.29 | 2.5 | from 5-bromopentanoyl |
| | | | | | | chloride 0 C.-rt, followed |
| | | | | | | by Suzuki coupling |
| 57 | 413.94 | 413, 415 | 97 | 1.24 | 2.5 | from 5-bromopentanoyl |
| | | | | | | chloride 0 C.-rt, followed |
| | | | | | | by Suzuki coupling |
| 58 | 444.57 | 445 | 99 | 1.23 | 2.5 | from 5-bromopentanoyl |
| | | | | | | chloride 0 C.-rt, followed |
| | | | | | | by Suzuki coupling |
| 59 | 470.05 | 469, 471 | 94 | 1.47 | 2.5 | from 5-bromopentanoyl |
| | | | | | | chloride 0 C.-rt, followed |
| | | | | | | by Suzuki coupling |
| 60 | 385.52 | 386 | 91 | 1.15 | 2.5 | from 5-bromopentanoyl |
| | | | | | | chloride 0 C.-rt, followed |
| | | | | | | by Suzuki coupling |
| 61 | 352.47 | 353 | 100 | 0.92 | 2.5 | from 5-bromopentanoyl |
| | | | | | | chloride 0 C.-rt, followed |
| | | | | | | by Suzuki coupling |
| 62 | 381.51 | 382 | 93 | 1.21 | 2.5 | from 5-bromopentanoyl |
| | | | | | | chloride 0 C.-rt, followed |
| | | | | | | by Suzuki coupling |
| 63 | 337.46 | 338 | 100 | 1.3 | 2.5 | from 5-bromopentanoyl |
| | | | | | | chloride 0 C.-rt, followed |
| | | | | | | by Suzuki coupling |
| 64 | 337.46 | 338 | 99 | 0.65 | 10 | from 5-bromopentanoyl |
| | | | | | | chloride 0 C.-rt, followed |
| | | | | | | by Suzuki coupling |
| 65 | 444.57 | 445 | 96 | 1.31 | 2.5 | from 5-bromopentanoyl |
| | | | | | | chloride 0 C.-rt, followed |
| | | | | | | by Suzuki coupling |
| 66 | 492.65 | 493 | 100 | 1.11 | 2.5 | from 5-bromopentanoyl |
| | | | | | | chloride 0 C.-rt, followed |
| | | | | | | by Suzuki coupling |
| 67 | 408.54 | 409 | 100 | 0.93 | 2.5 | from 5-bromopentanoyl |
| | | | | | | chloride 0 C.-rt, followed |
| | | | | | | by Suzuki coupling |
| 68 | 393.52 | 394 | 98 | 1.32 | 2.5 | from 5-bromopentanoyl |
| | | | | | | chloride 0 C.-rt, followed |
| | | | | | | by Suzuki coupling |
| 69 | 337.46 | 338 | 100 | 1.35 | 2.5 | from 5-bromopentanoyl |
| | | | | | | chloride 0 C.-rt, followed |
| | | | | | | by Suzuki coupling |
| 70 | 339.27 | 339, 341 | 100 | 1.49 | 2.5 | reductive alkylation on |
| | | | | | | N-(4-oxo- |
| | | | | | | butyl)bromobenzamide |
| | | | | | | followed by Suzuki |
| | | | | | | coupling |
| 71 | 415.48 | 416 | 95 | 1.23 | 2.5 | from 5-bromopentanoyl |
| | | | | | | chloride 0 C.-rt, followed |
| | | | | | | by Suzuki coupling |
| 72 | 372.45 | 373 | 100 | 1.74 | 2.5 | from 5-bromopentanoyl |
| | | | | | | chloride 0 C.-rt, followed |
| | | | | | | by Suzuki coupling |
| 73 | 342.50 | 343 | 100 | 1.67 | 2.5 | from 5-bromopentanoyl |
| | | | | | | chloride 0 C.-rt, followed |
| | | | | | | by Suzuki coupling |
| 74 | 357.51 | 358 | 95 | 1.19 | 2.5 | from 5-bromopentanoyl |
| | | | | | | chloride 0 C.-rt, followed |
| | | | | | | by Suzuki coupling |
| 75 | 415.48 | 416 | 98 | 1.22 | 2.5 | from 5-bromopentanoyl |
| | | | | | | chloride 0 C.-rt, followed |
| | | | | | | by Suzuki coupling |
| 76 | 379.50 | 380 | 99 | 2.41 | 10 | from 5-bromopentanoyl |
| | | | | | | chloride 0 C.-rt, followed |
| | | | | | | by Suzuki coupling |
| 77 | 409.52 | 410 | 94 | 1.18 | 2.5 | from 5-bromopentanoyl |
| | | | | | | chloride 0 C.-rt, followed |
| | | | | | | by Suzuki coupling |
| 78 | 409.52 | 410 | 92 | 1.19 | 2.5 | from 5-bromopentanoyl |
| | | | | | | chloride 0 C.-rt, followed |
| | | | | | | by Suzuki coupling |
| 79 | 486.61 | 487 | 96 | 1.19 | 2.5 | from 5-bromopentanoyl |
| | | | | | | chloride 0 C.-rt, followed |
| | | | | | | by Suzuki coupling |
| 80 | 387.47 | 388 | 93 | 1.22 | 2.5 | from 5-bromopentanoyl |
| | | | | | | chloride 0 C.-rt, followed |
| | | | | | | by Suzuki coupling |
| 81 | 379.54 | 380 | 91 | 1.4 | 2.5 | from 5-bromopentanoyl |
| | | | | | | chloride 0 C.-rt, followed |
| | | | | | | by Suzuki coupling |
| 82 | 364.52 | 365 | 100 | 1.9 | 2.5 | from 5-bromopentanoyl |
| | | | | | | chloride 0 C.-rt, followed |
| | | | | | | by Suzuki coupling |
| 83 | 372.45 | 373 | 100 | 1.73 | 2.5 | from 5-bromopentanoyl |
| | | | | | | chloride 0 C.-rt, followed |
| | | | | | | by Suzuki coupling |
| 84 | 436.55 | 437 | 92 | 0.98 | 2.5 | from 5-bromopentanoyl |
| | | | | | | chloride 0 C.-rt, followed |
| | | | | | | by Suzuki coupling |
| 85 | 413.94 | 413, 415 | 98 | 1.27 | 2.5 | from 5-bromopentanoyl |
| | | | | | | chloride 0 C.-rt, followed |
| | | | | | | by Suzuki coupling |
| 86 | 387.47 | 388 | 100 | 1.24 | 2.5 | from 5-bromopentanoyl |
| | | | | | | chloride 0 C.-rt, followed |
| | | | | | | by Suzuki coupling |
| 87 | 366.50 | 367 | 100 | 1.69 | 2.5 | rom 5-bromopentanoyl |
| | | | | | | chloride 0 C.-rt, followed |
| | | | | | | by Suzuki coupling |
| 88 | 366.50 | 367 | 99 | 2.42 | 10 | from 5-bromopentanoyl |
| | | | | | | chloride 0 C.-rt, followed |
| | | | | | | by Suzuki coupling |
| 89 | 385.50 | 386 | 94 | 2.06 | 10 | acid-amine coupling |
| | | | | | | with CDI, room temp. |
| 90 | 427.54 | 428, 485 | 100 | 1.29 | 2.5 | reductive alkylation on |
| | | | | | | N-(4-oxo- |
| | | | | | | butyl)bromobenzamide |
| | | | | | | followed by Suzuki |
| | | | | | | coupling |
| 91 | 423.55 | 424 | 100 | 1.17 | 2.5 | reductive alkylation on |
| | | | | | | N-(4-oxo- |
| | | | | | | butyl)bromobenzamide |
| | | | | | | followed by Suzuki |
| | | | | | | coupling |
| 92 | 427.97 | 428, 430 | 95 | 1.24 | 2.5 | reductive alkylation on |
| | | | | | | N-(4-oxo- |
| | | | | | | butyl)bromobenzamide |
| | | | | | | followed by Suzuki |
| | | | | | | coupling |
| 93 | 474.59 | 475 | 98 | 3.7 | 10 | acid-amine coupling |
| | | | | | | with CDI, room temp. |
| 94 | 508.05 | 508 | 98 | 4.31 | 10 | acid-amine coupling |
| | | | | | | with CDI, room temp. |
| 95 | 339.27 | 339, 341 | 99 | 2.74 | 10 | from 5-bromopentanoyl |
| | | | | | | chloride, 0 C.-rt |
| 96 | 436.55 | 437 | 100 | 2.74 | 10 | acid-amine coupling |
| | | | | | | with CDI, 60 C. |
| 97 | 436.55 | 437 | 100 | 2.86 | 10 | acid-amine coupling |
| | | | | | | with CDI, 60 C. |
| 98 | 356.26 | 356/358 | 100 | 1.54 | 10 | urea synthesis |
| 99 | 459.58 | 460 | 95 | 1.21 | 10 | urea synthesis followed |
| | | | | | | by cross-coupling in |
| | | | | | | microwave |
| 100 | 488.62 | 489 | 97 | 2.69 | 10 | urea synthesis followed |
| | | | | | | by cross-coupling in |
| | | | | | | microwave |
| 101 | 337.46 | 338 | 100 | 2.25 | 10 | reductive alkylation of |
| | | | | | | 4-oxobutylbenzamide |
| 102 | 354.29 | 356 | 100 | 1.73 | 10 | urea synthesis |
| 103 | 515.65 | 516 | 100 | 2.22 | 10 | urea synthesis followed |
| | | | | | | by cross-coupling in |
| | | | | | | microwave |
| 104 | 493.04 | 493 | 93 | 3.77 | 10 | urea synthesis followed |
| | | | | | | by cross-coupling in |
| | | | | | | microwave |
| 105 | 465.54 | 466 | 100 | 2.39 | 10 | urea synthesis followed |
| | | | | | | by cross-coupling in |
| | | | | | | microwave |
| 106 | 489.61 | 490 | 92 | 2.3 | 10 | urea synthesis followed |
| | | | | | | by Suzuki under thermal |
| | | | | | | heating |
| 107 | 396.48 | 397 | 92 | 2.27 | 10 | urea synthesis followed |
| | | | | | | by cross-coupling in |
| | | | | | | microwave |
| 108 | 383.48 | 384 | 98 | 3.01 | 10 | urea synthesis followed |
| | | | | | | by cross-coupling in |
| | | | | | | microwave |
| 109 | 354.45 | 355 | 100 | 0.58 | 10 | urea synthesis followed |
| | | | | | | by cross-coupling in |
| | | | | | | microwave |
| 110 | 389.44 | 390 | 100 | 3.15 | 10 | urea synthesis followed |
| | | | | | | by cross-coupling in |
| | | | | | | microwave |
| 111 | 410.51 | 411 | 90 | 2.5 | 10 | urea synthesis followed |
| | | | | | | by cross-coupling in |
| | | | | | | microwave |
| 112 | 364.52 | 365 | 100 | 3.63 | 10 | from 5-bromopentanoyl |
| | | | | | | chloride 0 C.-rt, followed |
| | | | | | | by Suzuki coupling |
| 113 | 394.55 | 395 | 100 | 3.64 | 10 | from 5-bromopentanoyl |
| | | | | | | chloride 0 C.-rt, followed |
| | | | | | | by Suzuki coupling |
| 114 | 352.47 | 353.39 | 98 | 2.7 | 10 | from 5-bromopentanoyl |
| | | | | | | chloride 0 C.-rt, followed |
| | | | | | | by Suzuki coupling |
| 115 | 404.47 | 405 | 98 | 3.54 | 10 | from 5-bromopentanoyl |
| | | | | | | chloride 0 C.-rt, followed |
| | | | | | | by Suzuki coupling |
| 116 | 390.44 | 391 | 98 | 3.48 | 10 | from 5-bromopentanoyl |
| | | | | | | chloride 0 C.-rt, followed |
| | | | | | | by Suzuki coupling |
| 117 | 338.44 | 339.35 | 100 | 2.65 | 10 | from 5-bromopentanoyl |
| | | | | | | chloride 0 C.-rt, followed |
| | | | | | | by Suzuki coupling |
| 118 | 301.38 | 302 | 98 | 2.37 | 10 | acid-amine coupling |
| | | | | | | with CDI, 60 C. |
| 119 | 301.38 | 302 | 99 | 2.00/2.07 | 10 | acid-amine coupling |
| | | | | | | with CDI, 60 C. |
| 120 | 301.38 | 302 | 100 | 1.79 | 10 | acid-amine coupling |
| | | | | | | with CDI, 60 C. |
| 121 | 406.52 | 407 | 100 | 2.90 | 10 | acid-amine coupling |
| | | | | | | with CDI, 60 C. |
| 122 | 406.52 | 407 | 95 | 2.76 | 10 | acid-amine coupling |
| | | | | | | with CDI, 60 C. |
| 123 | 412.48 | 413 | 100 | 2.89 | 10 | acid-amine coupling |
| | | | | | | with CDI, 60 C. |
| 124 | 398.54 | 399 | 100 | 2.89 | 10 | acid-amine coupling |
| | | | | | | with CDI, 60 C. |
| 125 | 379.50 | 380 | 99 | 1.61 | 10 | from 5-bromopentanoyl |
| | | | | | | chloride 0 C.-rt, followed |
| | | | | | | by Suzuki coupling |
| 126 | 365.47 | 366 | 99 | 1.37 | 10 | from 5-bromopentanoyl |
| | | | | | | chloride 0 C.-rt, followed |
| | | | | | | by Suzuki coupling |
| 127 | 358.42 | 359 | 97 | 2.41 | 10 | from 5-bromopentanoyl |
| | | | | | | chloride 0 C.-rt, followed |
| | | | | | | by Suzuki coupling |
| 128 | 356.89 | 357 | 95 | 2.49 | 10 | from 5-bromopentanoyl |
| | | | | | | chloride 0 C.-rt, followed |
| | | | | | | by Suzuki coupling |
| 129 | 352.47 | 353 | 98 | 2.25 | 10 | from 5-bromopentanoyl |
| | | | | | | chloride 0 C.-rt, followed |
| | | | | | | by Suzuki coupling |
| 130 | 325.24 | 325, 327 | 99 | 1.71 | 10 | from 5-bromopentanoyl |
| | | | | | | chloride, 0 C.-rt |
| 131 | 380.52 | 381 | 98 | 3.45 | 10 | from 5-bromopentanoyl |
| | | | | | | chloride 0 C.-rt, followed |
| | | | | | | by Suzuki coupling |
| 132 | 358.42 | 359 | 100 | 3.19 | 10 | from 5-bromopentanoyl |
| | | | | | | chloride 0 C.-rt, followed |
| | | | | | | by Suzuki coupling |
| 133 | 366.50 | 367 | 99 | 3.39 | 10 | from 5-bromopentanoyl |
| | | | | | | chloride 0 C.-rt, followed |
| | | | | | | by Suzuki coupling |
| 134 | 396.52 | 397 | 99 | 3.44 | 10 | from 5-bromopentanoyl |
| | | | | | | chloride 0 C.-rt, followed |
| | | | | | | by Suzuki coupling |
| 135 | 374.42 | 375 | 95 | 3.19 | 10 | from 5-bromopentanoyl |
| | | | | | | chloride 0 C.-rt, followed |
| | | | | | | by Suzuki coupling |
| 136 | 354.44 | 355.34 | 99 | 2.48 | 10 | from 5-bromopentanoyl |
| | | | | | | chloride 0 C.-rt, followed |
| | | | | | | by Suzuki coupling |
| 137 | 372.89 | 373 | 96 | 3.2 | 10 | from 5-bromopentanoyl |
| | | | | | | chloride 0 C.-rt, followed |
| | | | | | | by Suzuki coupling |
| 138 | 395.49 | 396 | 97 | 2.49 | 10 | from 5-bromopentanoyl |
| | | | | | | chloride 0 C.-rt, followed |
| | | | | | | by Suzuki coupling |
| 139 | 339.43 | 340 | 99 | double | 10 | from 5-bromopentanoyl |
| | | | | peak | | chloride 0 C.-rt, followed |
| | | | | 0.57- | | by Suzuki coupling |
| | | | | 1.19 | | |
| 140 | 374.42 | 375 | 97 | 3.14 | 10 | from 5-bromopentanoyl |
| | | | | | | chloride 0 C.-rt, followed |
| | | | | | | by Suzuki coupling |
| 141 | 381.47 | 382 | 95 | 2.29 | 10 | from 5-bromopentanoyl |
| | | | | | | chloride 0 C.-rt, followed |
| | | | | | | by Suzuki coupling |
| 142 | 368.47 | 369 | 100 | 3.05 | 10 | from 5-bromopentanoyl |
| | | | | | | chloride 0 C.-rt, followed |
| | | | | | | by Suzuki coupling |
| 143 | 437.53 | 438 | 100 | 1.95 | 10 | urea synthesis followed |
| | | | | | | by cross-coupling in |
| | | | | | | microwave |
| 144 | 381.51 | 382 | 96 | 2.99 | 10 | urea synthesis followed |
| | | | | | | by cross-coupling in |
| | | | | | | microwave |
| 145 | 424.54 | 425 | 92 | 2.7 | 10 | urea synthesis followed |
| | | | | | | by cross-coupling in |
| | | | | | | microwave |
| 146 | 518.65 | 519 | 94 | 3.33 | 10 | urea synthesis followed |
| | | | | | | by cross-coupling in |
| | | | | | | microwave |
| 147 | 428.95 | 429 | 100 | 2.97 | 10 | urea synthesis followed |
| | | | | | | by cross-coupling in |
| | | | | | | microwave |
| 148 | 395.50 | 396 | 95 | 0.52 | 10 | urea synthesis followed |
| | | | | | | by cross-coupling in |
| | | | | | | microwave |
| 149 | 545.67 | 546 | 96 | 2.87 | 10 | urea synthesis followed |
| | | | | | | by cross-coupling in |
| | | | | | | microwave |
| 150 | 408.54 | 409 | 96 | 2.37 | 10 | urea synthesis followed |
| | | | | | | by cross-coupling in |
| | | | | | | microwave |
| 151 | 451.56 | 452 | 92 | 2.16 | 10 | urea synthesis followed |
| | | | | | | by cross-coupling in |
| | | | | | | microwave |
| 152 | 383.48 | 384 | 100 | 3.05 | 10 | urea synthesis followed |
| | | | | | | by cross-coupling in |
| | | | | | | microwave |
| 153 | 521.60 | 522 | 100 | 3.27 | 10 | urea synthesis followed |
| | | | | | | by cross-coupling in |
| | | | | | | microwave |
| 154 | 323.43 | 324 | 99 | 0.55 | 10 | from 5-bromopentanoyl |
| | | | | | | chloride 0 C.-rt, followed |
| | | | | | | by Suzuki coupling |
| 155 | 352.47 | 353 | 99 | 2.91 | 10 | from 5-bromopentanoyl |
| | | | | | | chloride 0 C.-rt, followed |
| | | | | | | by Suzuki coupling |
| 156 | 397.31 | 399 | 100 | 2.39 | 10 | urea synthesis |
| 157 | 453.42 | 455 | 100 | 2.74 | 10 | urea synthesis |
| 158 | 475.58 | 476 | 96 | 2.51 | 10 | acid-amine coupling |
| | | | | | | with CDI, room temp. |
| 159 | 498.62 | 499 | 100 | 3.18 | 10 | acid-amine coupling |
| | | | | | | with CDI, room temp. |
| 160 | 530.66 | 531, 266 | 100 | 2.81 | 10 | acid-amine coupling |
| | | | | | | with CDI, room temp. |
| 161 | 361.48 | 362 | 100 | 2.66 | 10 | acid-amine coupling |
| | | | | | | with CDI, room temp. |
| 162 | 460.61 | 461 | 100 | 2.84 | 10 | acid-amine coupling |
| | | | | | | with CDI, room temp. |
| 163 | 460.61 | 461 | 100 | 2.88 | 10 | acid-amine coupling |
| | | | | | | with CDI, room temp. |
| 164 | 492.65 | 493 | 100 | 2.58 | 10 | acid-amine coupling |
| | | | | | | with CDI, room temp. |
| 165 | 404.50 | 405 | 99 | 2.35 | 10 | acid-amine coupling |
| | | | | | | with CDI, room temp. |
| 166 | 350.50 | 351 | 98 | 3.25 | 10 | from 5-bromopentanoyl |
| | | | | | | chloride 0 C.-rt, followed |
| | | | | | | by Suzuki coupling |
| 167 | 368.47 | 369 | 94 | 3.01 | 10 | from 5-bromopentanoyl |
| | | | | | | chloride 0 C.-rt, followed |
| | | | | | | by Suzuki coupling |
| 168 | 461.52 | 462.3 | 100 | 3.07 | 10 | from 5-bromopentanoyl |
| | | | | | | chloride 0 C.-rt, followed |
| | | | | | | by Suzuki coupling |
| 169 | 421.58 | 422.33 | 100 | 3.23 | 10 | from 5-bromopentanoyl |
| | | | | | | chloride 0 C.-rt, followed |
| | | | | | | by Suzuki coupling |
| 170 | 451.60 | 452.35 | 100 | 3.14 | 10 | from 5-bromopentanoyl |
| | | | | | | chloride 0 C.-rt, followed |
| | | | | | | by Suzuki coupling |
| 171 | 409.52 | 410.31 | 96 | 2.19 | 10 | from 5-bromopentanoyl |
| | | | | | | chloride 0 C.-rt, followed |
| | | | | | | by Suzuki coupling |
| 172 | 427.97 | 428.25 | 95 | 2.98 | 10 | from 5-bromopentanoyl |
| | | | | | | chloride 0 C.-rt, followed |
| | | | | | | by Suzuki coupling |
| 173 | 450.47 | 451.31 | 99 | 2.2 | 10 | from 5-bromopentanoyl |
| | | | | | | chloride 0 C.-rt, followed |
| | | | | | | by Suzuki coupling |
| 174 | 429.50 | 430.3 | 100 | 2.89 | 10 | from 5-bromopentanoyl |
| | | | | | | chloride 0 C.-rt, followed |
| | | | | | | by Suzuki coupling |
| 175 | 436.55 | 437.32 | 100 | 2 | 10 | from 5-bromopentanoyl |
| | | | | | | chloride 0 C.-rt, followed |
| | | | | | | by Suzuki coupling |
| 176 | 423.55 | 424.37 | 99 | 2.77 | 10 | from 5-bromopentanoyl |
| | | | | | | chloride 0 C.-rt, followed |
| | | | | | | by Suzuki coupling |
| 177 | 423.55 | 424.33 | 100 | 2.85 | 10 | from 5-bromopentanoyl |
| | | | | | | chloride 0 C.-rt, followed |
| | | | | | | by Suzuki coupling |
| 178 | 396.53 | 397 | 93 | 2.4 | 10 | urea synthesis followed |
| | | | | | | by cross-coupling in |
| | | | | | | microwave |
| 179 | 396.53 | 397 | 100 | 2.39 | 10 | urea synthesis followed |
| | | | | | | by cross-coupling in |
| | | | | | | microwave |
| 180 | 400.94 | 401 | 99 | 2.54 | 10 | urea synthesis followed |
| | | | | | | by cross-coupling in |
| | | | | | | microwave |
| 181 | 423.55 | 424 | 100 | 1.85 | 10 | urea synthesis followed |
| | | | | | | by cross-coupling in |
| | | | | | | microwave |
| 182 | 493.64 | 494 | 100 | 2.39 | 10 | urea synthesis followed |
| | | | | | | by cross-coupling in |
| | | | | | | microwave |
| 183 | 480.64 | 481 | 96 | 3.05 | 10 | urea synthesis followed |
| | | | | | | by cross-coupling in |
| | | | | | | microwave |
| 184 | 480.64 | 481 | 91 | 3.19 | 10 | urea synthesis followed |
| | | | | | | by cross-coupling in |
| | | | | | | microwave |
| 185 | 485.06 | 485 | 96 | 3.38 | 10 | urea synthesis followed |
| | | | | | | by cross-coupling in |
| | | | | | | microwave |
| 186 | 451.60 | 452 | 100 | 1.64 | 10 | urea synthesis followed |
| | | | | | | by cross-coupling in |
| | | | | | | microwave |
| 187 | 507.67 | 508 | 97 | 2.64 | 10 | urea synthesis followed |
| | | | | | | by cross-coupling in |
| | | | | | | microwave |
| 188 | 486.60 | 487 | 95 | 3.4 | 10 | urea synthesis followed |
| | | | | | | by cross-coupling in |
| | | | | | | microwave |
| 189 | 387.47 | 388 | 96 | 3.08 | 10 | urea synthesis followed |
| | | | | | | by cross-coupling in |
| | | | | | | microwave |
| 190 | 430.49 | 431 | 97 | 2.89 | 10 | urea synthesis followed |
| | | | | | | by cross-coupling in |
| | | | | | | microwave |
| 191 | 381.51 | 382 | 98 | 2.9 | 10 | urea synthesis followed |
| | | | | | | by cross-coupling in |
| | | | | | | microwave |
| 192 | 385.93 | 386 | 99 | 3.08 | 10 | urea synthesis followed |
| | | | | | | by cross-coupling in |
| | | | | | | microwave |
| 193 | 288.43 | 289 | 100 | 2.10 | 10 | from 5-bromopentanoyl |
| | | | | | | chloride in DCM/DMF, |
| | | | | | | 55 C. |
| 194 | 332.48 | 333 | 100 | 2.35 | 10 | from 5-bromopentanoyl |
| | | | | | | chloride in DCM/DMF, |
| | | | | | | 55 C. |
| 195 | 419.48 | 420 | 100 | 2.13 | 10 | from 5-bromopentanoyl |
| | | | | | | chloride 0 C.-rt, followed |
| | | | | | | by Suzuki coupling |
| 196 | 379.54 | 380 | 100 | 2.2 | 10 | from 5-bromopentanoyl |
| | | | | | | chloride 0 C.-rt, followed |
| | | | | | | by Suzuki coupling |
| 197 | 409.56 | 410 | 100 | 2.18 | 10 | from 5-bromopentanoyl |
| | | | | | | chloride 0 C.-rt, followed |
| | | | | | | by Suzuki coupling |
| 198 | 387.47 | 388 | 100 | 1.95 | 10 | from 5-bromopentanoyl |
| | | | | | | chloride 0 C.-rt, followed |
| | | | | | | by Suzuki coupling |
| 199 | 367.48 | 369.32 | 100 | 1.29 | 10 | from 5-bromopentanoyl |
| | 468 | | | | | chloride 0 C.-rt, followed |
| | | | | | | by Suzuki coupling |
| 200 | 381.51 | 382 | 100 | 1.83 | 10 | from 5-bromopentanoyl |
| | | | | | | chloride 0 C.-rt, followed |
| | | | | | | by Suzuki coupling |
| 201 | 404.47 | 405 | 97 | 2.69 | 10 | from 5-bromopentanoyl |
| | | | | | | chloride 0 C.-rt, followed |
| | | | | | | by Suzuki coupling |
| 202 | 394.55 | 395 | 97 | 2.75 | 10 | from 5-bromopentanoyl |
| | | | | | | chloride 0 C.-rt, followed |
| | | | | | | by Suzuki coupling |
| 203 | 372.45 | 373 | 100 | 2.49 | 10 | from 5-bromopentanoyl |
| | | | | | | chloride 0 C.-rt, followed |
| | | | | | | by Suzuki coupling |
| 204 | 366.50 | 367 | 100 | 2.35 | 10 | from 5-bromopentanoyl |
| | | | | | | chloride 0 C.-rt, followed |
| | | | | | | by Suzuki coupling |
| 205 | 280.79 | 281 | 100 | 1.67 | 10 | from 5-bromopentanoyl |
| | | | | | | chloride in DCM/DMF, |
| | | | | | | 55 C. |
| 206 | 294.82 | 295 | 100 | 1.78 | 10 | from 5-bromopentanoyl |
| | | | | | | chloride in DCM/DMF, |
| | | | | | | 55 C. |
| 207 | 318.45 | 319 | 100 | 2.24 | 10 | from 5-bromopentanoyl |
| | | | | | | chloride in DCM/DMF, |
| | | | | | | 55 C. |
| 208 | 334.45 | 335 | 100 | 2.18 | 10 | from 5-bromopentanoyl |
| | | | | | | chloride in DCM/DMF, |
| | | | | | | 55 C. |
| 209 | 290.40 | 291 | 100 | 1.27 | 10 | from 5-bromopentanoyl |
| | | | | | | chloride in DCM/DMF, |
| | | | | | | 55 C. |
| 210 | 304.43 | 305 | 100 | 1.99 | 10 | from 5-bromopentanoyl |
| | | | | | | chloride in DCM/DMF, |
| | | | | | | 55 C. |
| 211 | 302.45 | 303 | 100 | 2.24 | 10 | from 5-bromopentanoyl |
| | | | | | | chloride in DCM/DMF, |
| | | | | | | 55 C. |
| 212 | 317.47 | 318 | 100 | 0.38 | 10 | from 5-bromopentanoyl |
| | | | | | | chloride in DCM/DMF, |
| | | | | | | 55 C. |
| 213 | 331.50 | 332 | 100 | 0.40 | 10 | from 5-bromopentanoyl |
| | | | | | | chloride in DCM/DMF, |
| | | | | | | 55 C. |
| 214 | 318.41 | 319 | 100 | 1.24 | 10 | from 5-bromopentanoyl |
| | | | | | | chloride in DCM/DMF, |
| | | | | | | 55 C. |
| 215 | 353.30 | 353, 355 | 100 | 1.97 | 10 | from 5-bromopentanoyl |
| | | | | | | chloride - array |
| | | | | | | conditions |
| 216 | 353.30 | 353, 355 | 100 | 1.95 | 10 | from 5-bromopentanoyl |
| | | | | | | chloride - array |
| | | | | | | conditions |
| 217 | 323.43 | 324 | 100 | 0.85 | 10 | reductive alkylation of |
| | | | | | | 4-oxobutylbenzamide |
| 218 | 339.43 | 340 | 100 | 0.79 | 10 | reductive alkylation of |
| | | | | | | 4-oxobutylbenzamide |
| 219 | 314.35 | 315 | 100 | 2.10 | 10 | from 5-bromopentanoyl |
| | | | | | | chloride in DCM/DMF, |
| | | | | | | 55 C. |
| 220 | 328.37 | 329 | 100 | 1.98 | 10 | from 5-bromopentanoyl |
| | | | | | | chloride in DCM/DMF, |
| | | | | | | 55 C. |
| 221 | 314.35 | 315 | 100 | 2.18 | 10 | from 5-bromopentanoyl |
| | | | | | | chloride in DCM/DMF, |
| | | | | | | 55 C. |
| 222 | 330.35 | 331 | 100 | 2.05 | 10 | from 5-bromopentanoyl |
| | | | | | | chloride in DCM/DMF, |
| | | | | | | 55 C. |
| 223 | 328.37 | 329 | 100 | 2.09 | 10 | from 5-bromopentanoyl |
| | | | | | | chloride in DCM/DMF, |
| | | | | | | 55 C. |
| 224 | 325.24 | 325, 327 | 100 | 1.83 | 10 | from 5-bromopentanoyl |
| | | | | | | chloride in DCM/DMF, |
| | | | | | | 55 C. |
| 225 | 341.24 | 341, 343 | 100 | 1.63 | 10 | from 5-bromopentanoyl |
| | | | | | | chloride in DCM/DMF, |
| | | | | | | 55 C. |
| 226 | 301.38 | 302 | 100 | double | 10 | from 5-bromopentanoyl |
| | | | | peak | | chloride in DCM/DMF, |
| | | | | 0.42/0.69 | | 55 C. |
| 227 | 299.41 | 300 | 95 | 1.14 | 10 | from 5-bromopentanoyl |
| | | | | | | chloride in DCM/DMF, |
| | | | | | | 55 C. |
| 228 | 313.39 | 315 | 100 | 0.36 | 10 | from 5-bromopentanoyl |
| | | | | | | chloride in DCM/DMF, |
| | | | | | | 55 C. |
| 229 | 311.42 | 312 | 100 | 0.38 | 10 | from 5-bromopentanoyl |
| | | | | | | chloride in DCM/DMF, |
| | | | | | | 55 C. |
| 230 | 285.38 | 286 | 97 | 0.94 | 10 | from 5-bromopentanoyl |
| | | | | | | chloride in DCM/DMF, |
| | | | | | | 55 C. |
| 231 | 299.41 | 300 | 97 | 1.17 | 10 | from 5-bromopentanoyl |
| | | | | | | chloride in DCM/DMF, |
| | | | | | | 55 C. |
| 232 | 316.48 | 317 | 100 | 2.26 | 10 | from 5-bromopentanoyl |
| | | | | | | chloride in DCM/DMF, |
| | | | | | | 55 C. |
| 233 | 359.51 | 360.44 | 100 | 1.82 | 10 | from 5-bromopentanoyl |
| | | | | | | chloride - array |
| | | | | | | conditions |
| 234 | 308.85 | 309 | 100 | 1.81 | 10 | from 5-bromopentanoyl |
| | | | | | | chloride in DCM/DMF, |
| | | | | | | 55 C. |
| 235 | 351.87 | 352.32 | 100 | 1.28 | 10 | from 5-bromopentanoyl |
| | | | | | | chloride - array |
| | | | | | | conditions |
| 236 | 347.50 | 348 | 100 | 1.44 | 10 | from 5-bromopentanoyl |
| | | | | | | chloride in DCM/DMF, |
| | | | | | | 55 C. |
| 237 | 375.51 | 376 | 100 | 2.02 | 10 | from 5-bromopentanoyl |
| | | | | | | chloride in DCM/DMF, |
| | | | | | | 55 C. |
| 238 | 346.51 | 347 | 95 | 2.30 | 10 | from 5-bromopentanoyl |
| | | | | | | chloride in DCM/DMF, |
| | | | | | | 55 C. |
| 239 | 374.52 | 375.43 | 100 | 0.29 | 10 | from 5-bromopentanoyl |
| | | | | | | chloride - array |
| | | | | | | conditions |
| 240 | 374.52 | 375. | 100 | 0.29 | 10 | from 5-bromopentanoyl |
| | | | | | | chloride in DCM/DMF, |
| | | | | | | 55 C. |
| 241 | 345.52 | 346 | 100 | 0.32 | 10 | from 5-bromopentanoyl |
| | | | | | | chloride in DCM/DMF, |
| | | | | | | 55 C. |
| 242 | 388.55 | 389.41 | 100 | 0.30 | 10 | from 5-bromopentanoyl |
| | | | | | | chloride - array |
| | | | | | | conditions |
| 243 | 332.44 | 333 | 100 | 1.34 | 10 | from 5-bromopentanoyl |
| | | | | | | chloride in DCM/DMF, |
| | | | | | | 55 C. |
| 244 | 437.57 | 438 | 96 | 2.55 | 10 | from 5-bromopentanoyl |
| | | | | | | chloride 0 C.-rt, followed |
| | | | | | | by Suzuki coupling |
| 245 | 447.49 | 448 | 98 | 2.49 | 10 | from 5-bromopentanoyl |
| | | | | | | chloride 0 C.-rt, followed |
| | | | | | | by Suzuki coupling |
| 246 | 415.48 | 416 | 100 | 2.33 | 10 | from 5-bromopentanoyl |
| | | | | | | chloride 0 C.-rt, followed |
| | | | | | | by Suzuki coupling |
| 247 | 395.49 | 396.40 | 100 | 1.70 | 10 | from 5-bromopentanoyl |
| | | | | | | chloride 0 C.-rt, followed |
| | | | | | | by Suzuki coupling |
| 248 | 380.52 | 381 | 100 | 2.54 | 10 | from 5-bromopentanoyl |
| | | | | | | chloride 0 C.-rt, followed |
| | | | | | | by Suzuki coupling |
| 249 | 408.58 | 409 | 100 | 2.93 | 10 | from 5-bromopentanoyl |
| | | | | | | chloride 0 C.-rt, followed |
| | | | | | | by Suzuki coupling |
| 250 | 418.50 | 419 | 100 | 2.91 | 10 | from 5-bromopentanoyl |
| | | | | | | chloride 0 C.-45, followed |
| | | | | | | by Suzuki soupling |
| 251 | 386.48 | 387 | 100 | 2.74 | 10 | from 5-bromopentanoyl |
| | | | | | | chloride 0 C.-rt, followed |
| | | | | | | by Suzuki coupling |
| 252 | 366.50 | 367.42 | 100 | 2.05 | 10 | from 5-bromopentanoyl |
| | | | | | | chloride 0 C.-rt, followed |
| | | | | | | by Suzuki coupling |
| 253 | 393.52 | 394 | 100 | 1.77 | 10 | from 5-bromopentanoyl |
| | | | | | | chloride 0 C.-rt, followed |
| | | | | | | by Suzuki coupling |
| 254 | 407.55 | 408 | 100 | 2 | 10 | from 5-bromopentanoyl |
| | | | | | | chloride 0 C.-rt, followed |
| | | | | | | by Suzuki coupling |

Biological Activity

Cloning of alpha7 nicotinic acetylcholine receptor and generation of stable recombinant alpha7 nAChR expressing cell lines

Full length cDNAs encoding the alpha7 nicotinic acetylcholine receptor were cloned from a rat brain cDNA library using standard molecular biology techniques. Rat GH4C1 cells were then transfected with the rat receptor, cloned and analyzed for functional alpha7 nicotinic receptor expression employing a FLIPR assay to measure changes in intracellular calcium concentrations. Cell clones showing the highest calcium-mediated fluorescence signals upon agonist (nicotine) application were further subcloned and subsequently stained with Texas red-labelled a-bungarotoxin (BgTX) to analyse the level and homogeneity of alpha7 nicotinic acetylcholine receptor expression using confocal microscopy. Three cell lines were then expanded and one characterised pharmacologically (see Table 2 below) prior to its subsequent use for compound screening.

TABLE 2
Pharmacological characterisation of alpha7 nAChR stably
expressed in GH4C1 cells using the functional FLIPR assay

| Compound | EC₅₀ [microM] |
|---|---|
| Acetylcholine | 3.05 ± 0.08 (n = 4) |
| Choline | 24.22 ± 8.30 (n = 2) |
| Cytisine | 1.21 ± 0.13 (n = 5) |
| DMPP | 0.98 ± 0.47 (n = 6) |
| Epibatidine | 0.012 ± 0.002 (n = 7) |
| Nicotine | 1.03 ± 0.26 (n = 22) |

Development of a Functional FLIPR Assay for Primary Screening

A robust functional FLIPR assay (Z′=0.68) employing the stable recombinant GH4C1 cell line was developed to screen the alpha7 nicotinic acetylcholine receptor. The FLIPR system allows the measurements of real time Ca²⁺-concentration changes in living cells using a Ca²⁺ sensitive fluorescence dye (such as Fluo4). This instrument enables the screening for agonists and antagonists for alpha 7 nAChR channels stably expressed in GH4C1cells.

Cell Culture

GH4C1 cells stably transfected with rat-alpha7-nAChR (see above) were used. These cells are poorly adherent and therefore pretreatment of flasks and plates with poly-D-lysine was carried out. Cells are grown in 150 cm² T-flasks, filled with 30 ml of medium at 37° C. and 5% CO₂.

Data Analysis

EC₅₀ and IC₅₀ values were calculated using the IDBS XLfit4.1 software package employing a sigmoidal concentration-response (variable slope) equation:

Y=Bottom+((Top-Bottom)/(1+((EC₅₀/X) ̂ HillSlope))

Assay Validation

The functional FLIPR assay was validated with the alpha7 nAChR agonists nicotine, cytisine, DMPP, epibatidine, choline and acetylcholine. Concentration-response curves were obtained in the concentration range from 0.001 to 30 microM. The resulting EC₅₀ values are listed in Table 2 and the obtained rank order of agonists is in agreement with published data (Quik et al., 1997).

The assay was further validated with the specific alpha7 nAChR antagonist MLA (methyllycaconitine), which was used in the concentration range between 1 microM to 0.01 nM, together with a competing nicotine concentration of 10 microM. The IC₅₀ value was calculated as 1.31±0.43 nM in nine independent experiments.

Development of Functional FLIPR Assays for Selectivity Testing

Functional FLIPR assays were developed in order to test the selectivity of compounds against the alpha1 (muscular) and alpha3 (ganglionic) nACh receptors and the structurally related 5-HT3 receptor. For determination of activity at alpha1 receptors natively expressed in the rhabdomyosarcoma derived TE 671 cell line an assay employing membrane potential sensitive dyes was used, whereas alpha3 selectivity was determined by a calcium-monitoring assays using the native SH-SY5Y cell line. In order to test selectivity against the 5-HT3 receptor, a recombinant cell line was constructed expressing the human 5-HT3A receptor in HEK 293 cells and a calcium-monitoring FLIPR assay employed.

Screening of Compounds

The compounds were tested using the functional FLIPR primary screening assay employing the stable recombinant GH4C1 cell line expressing the alpha7 nAChR. Hits identified were validated further by generation of concentration-response curves. The potency of compounds from Examples 1-254 as measured in the functional FLIPR screening assay was found to range between 10 nM and 30 microM, with the majority showing a potency ranging between 10 nM and 10 microM.

The best exemplified compounds were also demonstrated to be selective against the alpha1 nACh, alpha3 nACh and 5HT3 receptors.

Cell based Assay of Neuroprotection

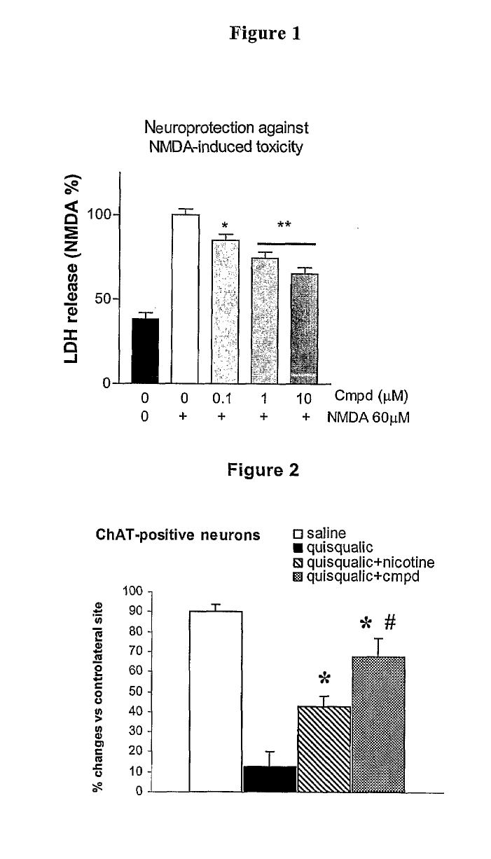

Neuroprotective activity of selected compounds was analyzed in an established cell-based assay of excitotoxicity induced by NMDA in mixed primary rat cortical neurons as described previously (Stevens et al, 2003). In brief, test compounds were added 24 h before NMDA application. Incubation with NMDA lasted 10 min or 24 h and cell mortality was assessed 24 h after application of the excitotoxic stimulus (see FIG. 1). Selected compounds (at concentrations ranging from 0.1 to 10 microM) reduced mortality on average by 50% and in some experiments a maximum of 80% neuroprotection was observed.

In vivo Neuroprotection Assay

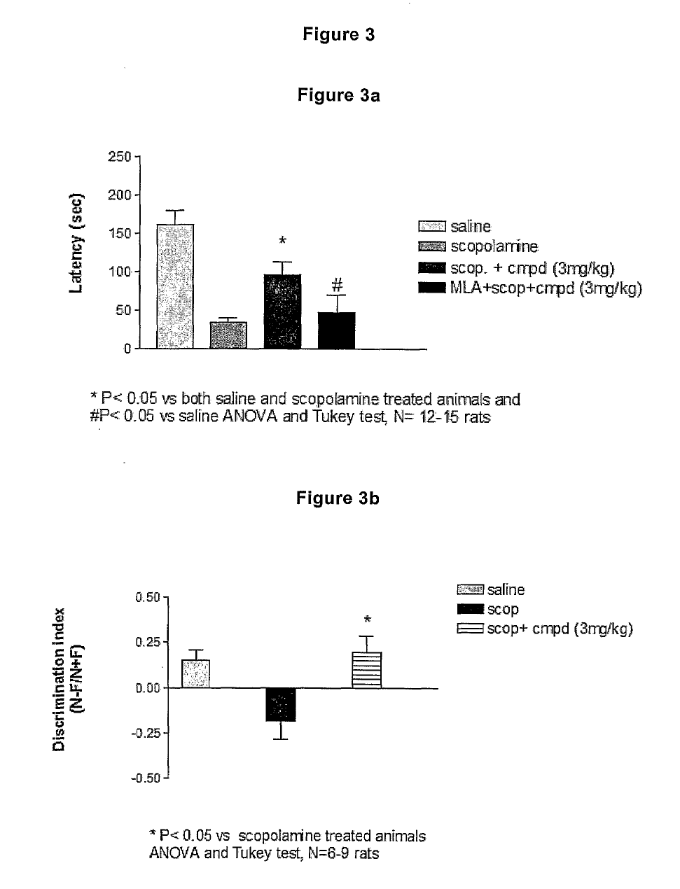

Neuroprotective activity of compounds was analyzed in an in vivo animal model of cholinergic degeneration induced by quisqualic acid injection in the nucleus basalis of rats. Subchronic treatment i.p. daily, for 7 days, with the compound at a dose of 3 mg/kg resulted in 60% reduction in the degeneration of cholinergic neurons as demonstrated by determination of the number of ChAT-positive neurons (a representative result is shown in FIG. 2).

Cognitive Behaviour

Cognitive behaviour was studied for selected compounds from example using the passive avoidance (PA) and object recognition (ORT) tests in order to test the capability to reverse scopolamine-induced amnesia in rats. The compounds showed mild to good cognitive improvement of short term-working and episodic memory by inducing significant reversion of scopolamine-induced amnesia in one or both tests (a representative result is shown in FIG. 3).

### REFERENCES

1. Prendergast, M. A., Harris, B. R., Mayer, S., Holley, R. C., Pauly, J. R., Littleton, J. M. (2001) Nicotine exposure reduces N-methyl-D-aspartate toxicity in the hippocampus: relation to distribution of the alpha7 nicotinic acetylcholine receptor subunit. Med.Sci.Monit. 7, 1153-1160.

2. Garrido, R., Mattson, M. P., Hennig, B., Toborek, M. (2001) Nicotine protects against arachidonic-acid-induced caspase activation, cytochrome c release and apoptosis of cultured spinal cord neurons. J.Neurochem. 76, 1395-1403.

3. Semba, J., Miyoshi, R., Kito, S. (1996) Nicotine protects against the dexamethasone potentiation of kainic acid-induced neurotoxicity in cultured hippocampal neurons. Brain Res. 735, 335-338.

4. Shimohama, S., Akaike, A., Kimura, J. (1996) Nicotine-induced protection against glutamate cytotoxicity. Nicotinic cholinergic receptor-mediated inhibition of nitric oxide formation. Ann.N.Y.Acad.Sci. 777, 356-361.

5. Akaike, A., Tamura, Y., Yokota, T., Shimohama, S., Kimura, J. (1994) Nicotine-induced protection of cultured cortical neurons against N-methyl-D-aspartate receptor-mediated glutamate cytotoxicity. Brain Res. 644, 181-187.

6. Yamashita, H., Nakamura, S. (1996) Nicotine rescues PC12 cells from death induced by nerve growth factor deprivation. Neurosci.Lett. 213, 145-147.

7. Shimohama, S., Greenwald, D. L., Shafron, D. H., Akaika, A., Maeda, T., Kaneko, S., Kimura, J., Simpkins, C. E., Day, A. L., Meyer, E. M. (1998) Nicotinic alpha 7 receptors protect against glutamate neurotoxicity and neuronal ischemic damage. Brain Res. 779, 359-363.

8. Socci, D. J., Arendash, G. W. (1996) Chronic nicotine treatment prevents neuronal loss in neocortex resulting from nucleus basalis lesions in young adult and aged rats. Mol.Chem.Neuropathol. 27, 285-305.

9. Rusted, J. M., Newhouse, P. A., Levin, E. D. (2000) Nicotinic treatment for degenerative neuropsychiatric disorders such as Alzheimer's disease and Parkinson's disease. Behav.Brain Res. 113, 121-129.

10. Kihara, T., Shimohama, S., Sawada, H., Kimura, J., Kume, T., Kochiyama, H., Maeda, T., Akaike, A. (1997) Nicotinic receptor stimulation protects neurons against beta-amyloid toxicity. Ann.Neurol. 42, 159-163.

11. Kihara, T., Shimohama, S., Sawada, H., Honda, K., Nakamizo, T., Shibasaki, H., Kume, T., Akaike, A. (2001) alpha 7 nicotinic receptor transduces signals to phosphatidylinositol 3-kinase to block A beta-amyloid-induced neurotoxicity. J.Biol. Chem. 276, 13541-13546.

12. Kelton, M. C., Kahn, H. J., Conrath, C. L., Newhouse, P. A. (2000) The effects of nicotine on Parkinson's disease. Brain Cogn 43, 274-282.

14. Kem, W. R. (2000) The brain alpha7 nicotinic receptor may be an important therapeutic target for the treatment of Alzheimer's disease: studies with DMXBA (GTS-21). Behav.Brain Res. 113, 169-181.

15. Dajas-Bailador, F. A., Lima, P. A., Wonnacott, S. (2000) The alpha7 nicotinic acetylcholine receptor subtype mediates nicotine protection against NMDA excitotoxicity in primary hippocampal cultures through a Ca(2+) dependent mechanism. Neuropharmacology 39, 2799-2807.

16. Strahlendorf, J. C., Acosta, S., Miles, R., Strahlendorf, H. K. (2001) Choline blocks AMPA-induced dark cell degeneration of Purkinje neurons: potential role of the alpha7 nicotinic receptor. Brain Res. 901, 71-78.

17. Utsugisawa, K., Nagane, Y., Obara, D., Tohgi, H. (2002) Overexpression of alpha7 nicotinic acetylcholine receptor prevents G1-arrest and DNA fragmentation in PC12 cells after hypoxia. J.Neurochem. 81, 497-505.

18. Jonnala, R. R., Terry, A. V., Jr., Buccafusco, J. J. (2002) Nicotine increases the expression of high affinity nerve growth factor receptors in both in vitro and in vivo. Life Sci. 70, 1543-1554.

19. Bencherif, M., Bane, A. J., Miller, C. H., Dull, G. M., Gatto, G. J. (2000) TC-2559: a novel orally active ligand selective at neuronal acetylcholine receptors. Eur.J.Pharmacol. 409, 45-55 Ref Type: Journal.

20. Donnelly-Roberts, D. L., Xue, I. C., Arneric, S. P., Sullivan, J. P. (1996) In vitro neuroprotective properties of the novel cholinergic channel activator (ChCA), ABT-418. Brain Res. 719, 36-44.

21 Meyer, E. M., Tay, E. T., Zoltewicz, J. A., Meyers, C., King, M. A., Papke, R. L., De Fiebre, C. M. (1998) Neuroprotective and memory-related actions of novel alpha-7 nicotinic agents with different mixed agonist/antagonist properties. J. Pharmacol.Exp.Ther. 284, 1026-1032.

22. Stevens, T. R., Krueger, S. K., Fizsimonds, R. M. and Picciotto, M. R. (2003) Neuroprotection by nicotine in mouse primary cortical cultures involves activation of calcineurin and L-type calcium channel inactivation. J. Neuroscience 23, 10093-10099.

23. Wang H, Yu M, Ochani M, Amella C A, Tanovic M, Susarla S, Li J H, Wang H, Yang H, Ulloa L, Al-Abed Y, Czura C J, Tracey K J: Nicotinic acetylcholine receptor alpha7 subunit is an essential regulator of inflammation. Nature 2003, 421:384-388.

1. A compound of general formula (I): 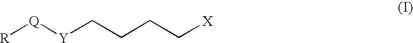
wherein:
Y is a group —CONH—; —NHCONH—; —NHCO—; —SO₂NH—; —NHSO₂—; —NHSO₂NH—; —OCONH; —NHCOO—;
Q is a 5 to 10-membered aromatic or heteroaromatic ring;
R is hydrogen; halogen; linear, branched or cyclic (C₁-C₆) alkyl, haloalkyl, alkoxy or acyl; hydroxy; cyano; nitro; mono- or di- (C₁-C₆) alkylamino, acylamino or alkylaminocarbonyl; carbamoyl; (C₆-C₁₀) aryl- or (C₁-C₆) alkylsulphonylamino; (C₆-C₁₀) aryl- or (C₁-C₆) alkylsulphamoyl; a 5 to
10-membered aromatic or heteroaromatic ring optionally substituted with: halogen; linear, branched or cyclic (C₁-C₃) alkyl, haloalkyl, alkoxy or acyl; hydroxy; cyano; nitro; amino; mono- or di- (C₁-C₆) alkylamino, acylamino or alkylaminocarbonyl groups; carbamoyl; (C₆-C₁₀) aryl- or (C₁-C₆) alkylsulphonylamino; (C₆-C₁₀) aryl- or (C₁-C₆) alkylsulphamoyl;
X is a group selected from 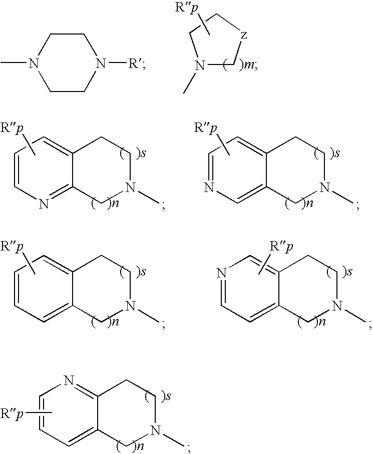
wherein
R′ represents (C₁-C₆) acyl; linear, branched or cyclic (C₁-C₆) alkyl; a —(CH₂)ⱼ—R′″ group, wherein j=0,1 and R′″ is a 5 to 10-membered aromatic or heteroaromatic ring optionally substituted with: halogen; hydroxy; cyano; nitro; (C₁-C₆) alkyl, haloalkyl, alkoxy, acyl, acylamino groups;
Z is CH₂, N or O;
m is an integer from 1 to 4;
n is 0 or 1;
s is 1 or 2;
p is 0, 1 or 2;
R″, independently from one another for p=2, represents hydrogen; halogen; hydroxy; cyano; nitro; linear, branched or cyclic (C₁-C₆) alkyl, haloalkyl, alkoxy, acyl; a —(CH₂)ⱼ—R′″ group, wherein n and R′″ are as above defined; carbamoyl; (C₆-C₁₀) aryl- or (C₁-C₃) alkylsulphonylamino; (C₆-C₁₀) aryl- or (C₁-C₃) alkylsulphamoyl; mono- or di-[linear, branched or cyclic (C₁-C₆) alkyl]aminocarbonyl;
salts, isomers, diastereomers or racemic mixtures thereof.

2. A compound according to claim 1, wherein
Y is —CONH—; —NHCO—; —NHCONH—;
Q is a 5 to 10-membered aromatic or heteroaromatic ring;
R is selected from the group consisting of hydrogen; halogen; linear, branched or cyclic (C₁-C₆) alkyl, alkoxy or alkylamino; trihaloalkyl; phenyl; naphthyl; pyridyl; pyrimidinyl; quinolinyl; isoquinolinyl; indolyl; thienyl; benzothienyl; furanyl; benzofuranyl; imidazolyl; benzoimidazolyl; pyrrolyl; optionally substituted as indicated in claim 1;
X is a group 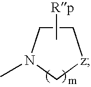
Z is CH₂, N or O
m is an integer from 1 to 4
p is 0, 1 or 2
R″, independently from one another for p=2, is selected from the group consisting of hydrogen; mono- or di-[linear, branched or cyclic (C₁-C₆) alkyl]aminocarbonyl; linear, branched or cyclic (C₁-C₆) alkyl, alkoxy, acyl.

3. A compound according to claim 2 wherein:
Y is —CONH(Q)-;
Q is a 5 to 10-membered aromatic or heteroaromatic ring;
R is selected from the group consisting of phenyl; naphthyl; pyridyl; pyrimidinyl; quinolinyl; isoquinolinyl; indolyl; thienyl; benzothienyl; furanyl; benzofuranyl; imidazolyl; benzoimidazolyl; pyrrolyl; optionally substituted as indicated in claim 1;
X is a group 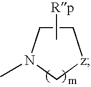
where
Z is CH₂, N or O
m is an integer from 1 to 4
p is 0, 1 or 2
R″, independently from one another when p=2, is selected from the group consisting of hydrogen; mono- or di-[linear, branched or cyclic (C₁-C₆) alkyl]aminocarbonyl; linear, branched or cyclic (C₁-C₆) alkyl, alkoxy, acyl;

4. A compound according to claim 2, wherein
Y is —NHCONH(Q)-;
Q is a 5 to 10-membered aromatic or heteroaromatic ring;
R is selected from the group consisting of halogen; linear, branched or cyclic (C₁-C₆) alkyl, alkoxy or alkylamino; haloalkyl; phenyl; naphthyl; pyridyl; pyrimidinyl; quinolinyl; isoquinolinyl; indolyl; thienyl; benzothienyl; furanyl; benzofuranyl; imidazolyl; benzoimidazolyl; pyrrolyl; optionally substituted as indicated in claim 1;
X is a group 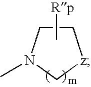
Z is CH₂, N or O
m is an integer from 1 to 4
p is 0, 1 or 2
R″, independently from one another when p=2, is selected from the group consisting of hydrogen; mono- or di-[linear, branched or cyclic (C₁-C₆) alkyl]aminocarbonyl; linear, branched or cyclic (C₁-C₆) alkyl, alkoxy, acyl;

5. A compound according to claim 2 whcrein
Y =—NHCO(Q)-;
Q is phenyl
R is selected from the group consisting of phenyl; naphthyl; pyridyl; pyrimidinyl; quinolinyl; isoquinolinyl; indolyl; thienyl; benzothienyl; furanyl; benzofuranyl; imidazolyl; benzoimidazolyl; pyrrolyl; optionally substituted as indicated in claim 1;
X is a group 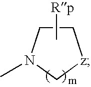
where
Z is CH₂, N or O
m is an integer from 1 to 4
p is 0, 1 or 2
R″, independently of one another when p=2, is selected from the group consisting of hydrogen; mono- or di-[linear, branched or cyclic (C₁-C₆) alkyl]aminocarbonyl; linear, branched or cyclic (C₁-C₆) alkyl, alkoxy, acyl.

6. A compound according to claim 1, wherein
Y is —CONH(Q)
Q is phenyl, indolyl
R is selected from the group consisting of halogen; phenyl; naphthyl; pyridyl; quinolinyl; isoquinolinyl; indolyl; thienyl; benzothienyl; furanyl; benzofuranyl; imidazolyl; benzoimidazolyl; pyrrolyl; optionally substituted as indicated in claim 1;
X is a group 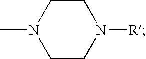
where R′ is a 5-10-membered aromatic or heteroaromatic ring optionally substituted with halogen or (C₁-C₆) alkoxy groups;

7. A compound according to claim 1 wherein
Y is —NHCONH(Q)
Q is phenyl, indolyl
R is selected from the group consisting of halogen; phenyl; naphthyl; pyridyl; quinolinyl; isoquinolinyl; indolyl; thienyl; benzothienyl; furanyl; benzofuranyl; imidazolyl; benzoimidazolyl; pyrrolyl; optionally substituted as indicated in claim 1;
X is a group 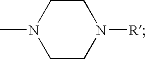
where R′ is a 6-membered aromatic or heteroaromatic ring optionally substituted with halogen or (C₁-C₆) alkoxy groups.

8. A compound according to claim 1, wherein
Y is —NHCO(Q);
Q is phenyl, pyridyl
R is selected from the group consisting of phenyl; naphthyl; pyridyl; quinolinyl; pyrimidinyl, isoquinolinyl; indolyl; thienyl; benzothienyl; furanyl; benzofuranyl; imidazolyl; benzoimidazolyl; pyrrolyl; optionally substituted as indicated in claim 1;
X is a group 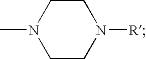
where R′ is a phenyl ring optionally substituted with halogen or (C₁-C₆) alkoxy groups.

9. A compound according to claim 8 wherein
Y is —NHCO(Q);
Q is phenyl
R is selected from the group consisting of phenyl; pyridyl; indolyl; pyrimidinyl; optionally substituted with: halogen; linear, branched or cyclic (C₁-C₃) alkyl, alkoxy or acyl; cyano; (C₁-C₆) alkylamino; acylamino; alkylaminocarbonyl groups; carbamoyl;
X is a group 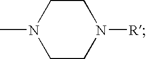
where R′ is a phenyl ring optionally substituted with halogen or (C₁-C₆) alkoxy groups.

10. A pharmaceutical composition containing a compound according to any one of claims 1-9, in combination with a pharmaceutically acceptable carrier or excipient.

11. A method for treating a neurological, psychiatric, cognitive, immunological or inflammatory disorder, which comprises administering to a subject in need thereof an effective amount of a compound according to any one of claims 1-9.

12. (canceled)

13. A method for the prevention or treatment of diseases, conditions or dysfunctions involving the alpha 7 nAChR, which comprises administering to a subject in need thereof an effective amount of a compound according to any one of claims 1-9.

14. A method according to claim 13, for the prevention or treatment of a neurodegenerative disease.

15. A method according to claim 14, wherein the neurodegenerative disease is Alzheimer's disease.

16. A method according to claim 14, wherein the neurodegenerative disease is schizophrenia.
